(19) United States
(12) Patent Application Publication
(10) Pub. No.: US 2008/0076701 A1
(43) Pub. Date: Mar. 27, 2008

(54) DICER SUBSTRATE RNA PEPTIDE CONJUGATES AND METHODS FOR RNA THERAPEUTICS

(75) Inventors: Steven C. Quay, Woodinville, WA (US); Paul Hickok Johnson, Snohomish, WA (US); Michael E. Houston, Sammamish, WA (US); Roger C. Adami, Snohomish, WA (US); Renata Fam, Kenmore, WA (US)

Correspondence Address: NASTECH PHARMACEUTICAL COMPANY INC, 3830 MONTE VILLA PARKWAY, BOTHELL, WA, 98021-7266, US

(73) Assignee: NASTECH PHARMACEUTICAL COMPANY INC., Bothell, WA (US)

(21) Appl. No.: 11/837,432

(22) Filed: Aug. 10, 2007

Related U.S. Application Data
(60) Provisional application No. 60/822,896, filed on Aug. 18, 2006.
(60) Provisional application No. 60/939,578, filed on May 22, 2007.
(60) Provisional application No. 60/945,868, filed on Jun. 22, 2007.

(51) Int. Cl. A61K 38/00; A61P 43/00; C07K 14/00

(52) U.S. Cl. 514/2; 530/350

(57) ABSTRACT

Dicer substrate RNA peptide conjugates comprising a double stranded ribonucleic acid (dsRNA) having a sense strand and an antisense strand, and a peptide, wherein the dicer substrate RNA is conjugated to the peptide, and compositions and methods of use thereof.

This application claims the benefit under 35 U.S.C. §119(e) of U.S. Provisional Application Nos. 60/822,896 filed Aug. 18, 2006, 60/939,578 filed May 22, 2007 and 60/945,868 filed Jun. 22, 2007, each of which is incorporated herein by reference in its entirety.

### BACKGROUND

RNA Interference (RNAi) refers to methods of sequence-specific post-transcriptional gene silencing which is mediated by a double-stranded RNA (dsRNA) called a short interfering RNA (siRNA). See Fire, et al, Nature 391:806, 1998, and Hamilton, et al., Science 286:950-951, 1999. RNAi is shared by diverse flora and phyla and is believed to be an evolutionarily-conserved cellular defense mechanism against the expression of foreign genes. See Fire, et al., Trends Genet. 15:358, 1999.

RNAi is therefore a ubiquitous, endogenous mechanism that uses small noncoding RNAs to silence gene expression. See Dykxhoorn, D. M. and J. Lieberman, Annu. Rev. Biomed. Eng. 8:377-402, 2006. RNAi can regulate important genes involved in cell death, differentiation, and development. RNAi may also protect the genome from invading genetic elements, encoded by transposons and viruses. When an siRNA is introduced into a cell, it binds to the endogenous RNAi machinery to disrupt the expression of mRNA containing complementary sequences with high specificity. Any disease-causing gene and any cell type or tissue can potentially be targeted. This technique has been rapidly utilized for gene-function analysis and drug-target discovery and validation. Harnessing RNAi also holds great promise for therapy, although introducing siRNAs into cells in vivo remains an important obstacle.

The mechanism of RNAi, although not yet fully characterized, is through cleavage of a target mRNA or via inhibition of mRNA translation (e.g., microRNA), which is also mediated by RISC (see below). The RNAi response involves an endonuclease complex known as the RNA-induced silencing complex (RISC), which mediates cleavage of a single-stranded RNA complementary to the antisense strand of the siRNA duplex. Cleavage of the target RNA takes place in the middle of the region complementary to the antisense strand of the siRNA duplex (Elbashir, et al., Genes Dev. 15:188, 2001).

RNAi may be accomplished by introducing siRNAs into cells or expressing an siRNA in cells via expression vector. Alternatively, dsRNA molecules may be introduced into cells, which are subsequently processed by an endogenous ribonuclease III enzyme called dicer. See Hamilton, et al., Science 286:950-951, 1999; Berstein, et al, Nature 409:363, 2001. An siRNA derived from dicer is typically about 21-23 nucleotides in overall length with about 19 base pairs duplexed. See Hamilton, et al., supra; Elbashir, et al., Genes Dev. 15:188, 2001. In essence, a long dsRNA can be introduced in a cell as a precursor of an siRNA.

The development of RNAi therapy, antisense therapy, and gene therapy, among others, has increased the need for effective means of introducing nucleic acid-based agents into cells. In general, nucleic acids are stable for only limited times in cells or plasma. However, nucleic acid-based agents can be stabilized in compositions and formulations which may be dispersed for cellular delivery.

What is needed are compositions and formulations for intracellular and in vivo delivery of a nucleic acid agent for use, ultimately, as a therapeutic, which exhibit relatively low toxicity, efficient intracellular delivery and permit double-stranded RNA to produce the response of RNA interference. Moreover, there is a need for compositions and methods for delivery of interfering RNAs to selected cells, tissues, or compartments to modulate gene expression in a manner that will alter a phenotype or disease state.

### SUMMARY

This disclosure satisfies these needs and fulfills additional objects and advantages by providing a range of novel structures, compositions and methods that employ an interfering ribonucleic acid, or a precursor thereof, with a delivery peptide. The precursor interfering ribonucleic acid may be a dicer substrate and the delivery peptide may be a natural or artificial polypeptide selected for its ability to enhance intracellular delivery or uptake of polynucleotides, interfering ribonucleic acids and their precursors.

In some embodiments, the delivery peptide may be a peptide containing the amino acid sequence SEQ ID NO:32 PN963 ESYSVYVYKVLKQ, or a variant thereof.

In some embodiments, a dicer substrate peptide conjugate can be a double stranded ribonucleic acid (dsRNA) having a sense strand and an antisense strand and a double-stranded region of from 25 to 30 base pairs, and a peptide comprising from about 5 to about 100 amino acids, wherein the dsRNA is conjugated to the peptide.

In some embodiments, a dicer substrate peptide conjugate has strands with lengths from about 25 to about 29 base pairs which the strands may have the same or different length. In some embodiments, a dicer substrate peptide conjugate has strands with lengths from about 25 to about 27 base pairs which may be the same or different.

One aspect of this disclosure is that a dicer substrate peptide conjugate can have the peptide conjugated to the 5′ end of the antisense strand or the sense strand. In other aspects, a dicer substrate peptide conjugate may have the peptide conjugated to the 3′ end of the sense strand or the antisense strand. Preferably, a dicer substrate peptide conjugate can have the peptide conjugated to the 5′ end of the antisense strand or the 3′ end of the sense strand.

A dicer substrate peptide conjugate of this disclosure can have an antisense strand complementary to a portion of a human mRNA of TNF-alpha.

In some embodiments, a dicer substrate peptide conjugate may have an antisense strand complementary to a portion of a gene of an influenza virus.

A dicer substrate peptide conjugate of this disclosure may include a dsRNA that contains one or more chemically-modified nucleotides.

In some respects, the dsRNA of a dicer substrate peptide conjugate can have a 2 nucleotide 3′ antisense strand overhang. In some embodiments, the dsRNA of a dicer substrate peptide conjugate may have a 2 nucleotide 3′ sense strand overhang. In another aspect, the dsRNA of a dicer substrate peptide conjugate may have no overhang.

This disclosure provides a dicer substrate conjugate which may contain a peptide having an amphipathic domain, a protein transduction domain, a fusogenic domain, or a nucleic acid binding domain. In some embodiments, the peptide of a dicer substrate peptide conjugate can be pegylated.

In further aspects, this disclosure includes a pharmaceutical composition containing a dicer substrate peptide conjugate and one or more carriers.

This disclosure provides a method for treating influenza in an animal by administering an effective amount of a dicer substrate peptide conjugate to the animal.

In some embodiments, this disclosure provides a method for treating inflammation associated with TNF-alpha in an animal by administering an effective amount of a dicer substrate peptide conjugate to the animal. The inflammation associated with TNF-alpha may occur in arthritis or psoriasis. This disclosure also provides a method for inhibiting expression of a TNF-alpha gene in an animal by administering an inhibiting amount of a dicer substrate peptide conjugate to the animal.

This brief summary, taken along with the description of drawings, detailed description, as well as the appended examples, claims, and drawings, as a whole, encompasses the disclosure.

### BRIEF DESCRIPTION OF THE DRAWINGS

FIG. 1: RP-HPLC analysis of dicer endonuclease processing kinetics for non-conjugated siRNA N163 duplex. (A) unprocessed N163 duplex, (B) incubation with dicer endonuclease for 1 hr, (C) incubation with dicer endonuclease for 2.5 hr, (D) incubation with dicer endonuclease for 5 hr, (E) incubation with dicer endonuclease for 7 hr.

FIG. 2: Chart of RP-HPLC analysis of dicer endonuclease processing kinetics for non-conjugated siRNA N163 duplex shown in FIG. 1.

FIG. 3: ESI-MS analysis of 7 hr dicer digestion of non-conjugated N163.

FIG. 4: ESI-MS analysis of dicer endonuclease processing for a conjugated siRNA having polypeptide PN857 conjugated to siRNA N163. (A) 8 hr control without dicer endonuclease present, (B) 8 hr dicer endonuclease digestion of conjugate.

### DETAILED DESCRIPTION

This disclosure relates generally to the fields of RNA interference, delivery of RNA therapeutics, and to the chemistry of biological conjugates. More particularly, this disclosure relates to compositions and formulations for ribonucleic acids, and their uses for medicaments and for delivery as therapeutics. This disclosure relates generally to methods of using ribonucleic acids in RNA interference for gene-specific inhibition of gene expression in mammals.

This disclosure provides a range of compositions, formulations and methods which include an interfering ribonucleic acid, or a precursor thereof, in combination with various components including peptides, lipids, and natural or synthetic polymers.

The compositions and formulations of this disclosure may be used for delivery of RNAi-inducing entities such as dsRNA, siRNA, shRNA, or RNAi-inducing vectors to cells, tissues, or organs in mammalian subjects, and may also be used for delivery of these agents to cells in culture.

In some aspects, this disclosure provides compositions containing one or more RNAi-inducing agents which are targeted to one or more target transcripts, along with one or more delivery components. Examples of delivery components include peptides, lipids, neutral lipids, anionic lipids, cationic lipids, liposomes, surfactants, and polymers.

This disclosure also provides methods for the delivery of one or more RNAi-inducing entities to cells, organs or tissues within an animal. In some embodiments, compositions containing an RNAi-inducing entity, one or more peptides, and optionally one or more lipids, are introduced by various routes to be transported within the body and taken up by cells in one or more organs or tissues, where expression of a target transcript can be modulated.

This disclosure provides pharmaceutically acceptable nucleic acid compositions useful for therapeutic delivery of nucleic acids and gene-silencing RNAs. In particular, this disclosure provides compositions and methods for in vitro and in vivo delivery of dsRNAs for decreasing, downregulating, or silencing the translation of a target nucleic acid sequence or expression of a gene. These compositions and methods may be used for prevention and/or treatment of diseases in a mammal.

In exemplary compositions and methods of this disclosure, a ribonucleic acid molecule such as a double-stranded RNA may be conjugated with a peptide to formulate a composition which can be administered to cells or subjects such as mammals. In some embodiments, this disclosure provides methods for delivering a dsRNA, siRNA, an shRNA, or a precursor thereof, intracellularly by contacting a nucleic acid-containing composition with a cell.

This disclosure provides novel compositions and methods that employ an interfering ribonucleic acid, or a precursor thereof, in combination with a delivery peptide. The delivery peptide may be a natural or artificial polypeptide selected for its ability to enhance intracellular delivery or uptake of polynucleotides, including interfering RNAs and their precursors.

This disclosure encompasses delivery peptides conjugated to dicer-active dsRNAs. As used herein, the term “dicer substrate” refers to a dicer-active dsRNA, which is a dsRNA that is capable of being processed by dicer ribonuclease. Dicer-active dsRNA peptide conjugates of this disclosure can be used as novel therapeutic pro-drug delivery systems in the treatment of disease. These dicer-active dsRNA peptide conjugates function analogous to a pro-drug or precursor siRNA in that upon delivery into a cell, the dsRNA peptide conjugate can be processed and cleaved by dicer, whereupon an siRNA is liberated that is capable of loading into the RISC complex. The liberated siRNA may then enter the RISC complex to effect post-transcriptional gene silencing. Thus, the dicer-active dsRNA peptide conjugate, and the dicer-liberated siRNA are RNAi-inducing agents.

As used herein, the terms “dsRNA,” “siRNA” and “siNA” when referring to an RNAi-inducing agent, also refer to precursor dicer substrates from which they may be derived.

Biological assessment indicates that a dicer-active dsRNA peptide conjugate, which is a precursor siRNA, represents a promising new approach for improving the delivery of RNA therapeutics into cells for the treatment of a wide range of diseases and disorders.

Within novel compositions of this disclosure, an interfering RNA may be conjugated to a delivery peptide to form its own composition for intracellular delivery of the interfering RNA or its precursor. Delivery of the interfering RNA or its precursor can be enhanced relative to delivery resulting from contacting a target cell with a naked interfering RNA that is not conjugated to a peptide. In some embodiments, an interfering RNA conjugate may be admixed with other excipients, carriers, vehicles, buffers, or solvents for delivery to a cell.

The compositions of this disclosure can form stable particles which may incorporate a dicer-active dsRNA peptide conjugate as an interfering RNA agent.

In some embodiments, compositions of this disclosure contain stable RNA-lipid particles having diameters from about 5 nm to about 400 nm. In some embodiments, the particles may have a uniform diameter of from about 10 nm to about 300 nm. In some embodiments, the particles may have a uniform diameter of from about 50 nm to about 150 nm.

In some embodiments, a composition of this disclosure may contain one or more lipids which are from about 0.5% to about 95% (mol %) of the total amount of lipid and delivery-enhancing components, including polymers.

In some embodiments, the delivery peptide may be an amphipathic amino acid sequence. For example, the peptide may have a plurality of non-polar or hydrophobic amino acid residues that form a hydrophobic sequence domain or motif which may be linked to a plurality of charged amino acid residues that form a charged sequence domain or motif, yielding an amphipathic peptide.

Peptides suitable to prepare dsRNA conjugates of this disclosure may have from about 5 to about 100 amino acids.

In some embodiments, the delivery peptide may include a protein transduction domain or motif. In general, a protein transduction domain is an amino acid sequence that is able to insert into, and preferably transit through, a membrane of a cell.

Examples of protein transduction domains include:

(SEQ ID NO: 1) (TAT)
KRRQRRR;
(SEQ ID NO: 2) (Penetratin)
RQIKIWFQNRRMKWKK;
(SEQ ID NO: 3) (VP22)
DAATATRGRSAASRPTERPRAPARSASRPRRPVD;
(SEQ ID NO: 4) (Kaposi FGF signal sequences)
AAVALLPAVLLALLAP,
(SEQ ID NO: 5)
AAVLLPVLLPVLLAAP;
(SEQ ID NO: 6) (Human β3 integrin signal sequence)
VTVLALGALAGVGVG;
(SEQ ID NO: 7) (gp41 fusion sequence)
GALFLGWLGAAGSTMGA;
(SEQ ID NO: 8) (Caiman crocodylus Ig(v) light
chain)
MGLGLHLLVLAAALQGA;
(SEQ ID NO: 9) (hCT-derived peptide)
LGTYTQDFNKFHTFPQTAIGVGAP;
(SEQ ID NO: 10) (Transportan)
GWTLNSAGYLLKINLKALAALAKKIL;
(SEQ ID NO: 11) (Loligomer)
TPPKKKRKVEDPKKKK;
(SEQ ID NO: 12)
RRRRRRR;
(SEQ ID NO: 13) (amphiphilic peptide)
KLALKLALKALKAALKLA.

In some embodiments, the delivery peptide may be a fusogenic peptide domain or motif In general, a fusogenic peptide is a peptide that can impart fusion activity to a biological material towards a membrane or cell. A fusogenic peptide may destabilize a lipid membrane, e.g., a plasma membrane or membrane surrounding an endosome, thereby inducing fusion. Fusogenic activity may be greater at lower pH. Exemplary fusogenic domains or motifs are found in a broad diversity of viral fusion proteins and in other proteins, e.g., fibroblast growth factor 4 (FGF-4).

Examples of fusogenic domains include:

(SEQ ID NO: 14) (Influenza HA2)
GLFGAIAGFIENGWEG;
(SEQ ID NO: 15) (Sendai F1)
FFGAVIGTIALGVATA;
(SEQ ID NO: 16) (Respiratory Syncytial virus F1)
FLGFLLGVGSAIASGV;
(SEQ ID NO: 17) (HIV gp41)
GVFVLGFLGFLATAGS;
(SEQ ID NO: 18) (Ebola GP2)
GAAIGLAWIPYFGPAA.

A protein transduction domain may be employed as a motif to facilitate entry of a nucleic acid agent into a cell through a plasma membrane. In some embodiments, a nucleic acid agent may be encapsulated in an endosome. The interior of an endosome may have a low pH so that a fusogenic peptide motif may destabilize the membrane of the endosome to allow release of the nucleic acid agent.

In some embodiments, the delivery peptide may include a nucleic acid binding domain or motif. Exemplary DNA binding domains include various “zinc finger” domains as described for DNA-binding regulatory proteins and other proteins identified in Table 1, below (see, e.g., Simpson, et al., J. Biol. Chem. 278:28011-28018, 2003).

TABLE 1

Exemplary Zinc Finger Motifs of DNA-binding Proteins
C₂H₂ Zinc finger motif

| | |
|---|---|
| | ....\|....\| ....\|....\| ....\|....\| ....\|....\| ....\|....\| ....\|....\|....\|....\| |
| | 665 675 685 695 705 715 |
| Sp1 | ACTCPYCKDS EGRGSG---- DPGKKKQHIC HIQGCGKVYG KTSHLRAHLR WHTGERPFMC |
| Sp2 | ACTCPNCKDG EKRS------ GEQGKKKHVC HIPDCGKTFR KTSLLRAHVR LHTGERPFVC |
| Sp3 | ACTCPNCKEG GGRGTN---- -LGKKKQHIC HIPGCGKVYG KTSHLRAHLR WHSGERPFVC |
| Sp4 | ACSCPNCREG EGRGSN---- EPGKKKQHIC HIEGCGKVYG KTSHLRAHLR WHTGERPFIC |
| DrosBtd | RCTCPNCTNE MSGLPPIVGP DERGRKQHIC HIPGCERLYG KASHLKTHLR WHTGERPFLC |
| DrosSp | TCDCPNCQEA ERLGPAGV-- HLRKKNIHSC HIPGCGKVYG KTSHLKAHLR WHTGERPFVC |
| CeT22C8.5 | RCTCPNCKAI KHG------- DRGSQHTHLC SVPGCGKTYK KTSHLRAHLR KHTGDRPFVC |
| Y40B1A.4 | PQISLKKKIF FFIFSNFR-- GDGKSRIHIC HL--CNKTYG KTSHLRAHLR GHAGNKPFAC |

The sequences shown in Table 1 for Sp1, Sp2, Sp3, Sp4, DrosBtd, DrosSp, CeT22C8.5, and Y40B1A.4, are herein assigned SEQ ID NOs: 19, 20, 21, 22, 23, 24, 25, and 26, respectively.

Table 1 shows a conservative zinc finger motif for double strand DNA binding which is characterized by the PROSITE pattern C-x(2,4)-C-x(12)-H-x(3)-H motif (SEQ ID NO:113). This motif can also be used to select and design additional delivery peptides.

Alternative DNA binding domains useful for constructing delivery peptides include, e.g., portions of the HIV TAT protein sequence.

Within exemplary embodiments of this disclosure, delivery peptides may be constructed by combining any of the foregoing structural elements, domains or motifs into a single polypeptide.

Examples of delivery peptides include Poly-Lys-Trp, 4:1, Mw 20,000-50,000; Poly-Orn-Trp, 4:1, Mw 20,000-50,000; fragments or variants of mellitin protein, and fragments or variants of a histone protein, e.g., histone H1, histone H2A, histone H2B, histone H3 or histone H4.

The delivery peptide may be pegylated, or covalently attached to a synthetic or natural polymer. The delivery peptide may be made up of L or D amino acids or mixtures thereof.

In general, an RNAi-inducing agent has two strands and four strand ends. In this disclosure, the RNAi-inducing agent may be conjugated at one or more of the four ends. In some embodiments, the conjugate is covalently attached to the nucleic acid molecule via a biodegradable linker. In some embodiments, a conjugate is attached at the 3′-end of either the sense strand or the antisense strand, or at the 3′-end of both strands of the nucleic acid molecule. In another embodiment, a conjugate is attached at the 5′-end of either the sense strand or the antisense strand, or at the 5′-end of both strands of the nucleic acid molecule. In some embodiments, a conjugate is attached at both the 3′-end and the 5′-end of the sense strand. In some embodiments, a conjugate is attached at both the 3′-end and the 5′-end of the antisense strand. In some embodiments, a conjugate is attached at both the 3′-end and the 5′-end of both the sense strand and the antisense strand. In some embodiments, a conjugate is attached to a nucleobase within the nucleic acid molecule. In some embodiments, a conjugate is attached to the “backbone” of the nucleic acid molecule. The backbone may be a sugar backbone, glycerol backbone (e.g., glycerol nucleic acid), peptidic backbone (e.g., peptide nucleic acid) or threose backbone (e.g., threose nucleic acid).

Preferably, a dicer substrate peptide conjugate can have the peptide conjugated to the 5′ end of the antisense strand or the 3′ end of the sense strand.

Examples of structures, linkers, and methods for making nucleic acid conjugates include conjugates and ligands described in Vargeese, et al., U.S. Patent Application Publication Nos. 20040110296, 20030130186, and 20040110296. As used herein, and among other meanings, the term “ligand” refers to molecules, groups, and/or moieties that can be attached to a nucleic acid molecule to form a conjugate.

Examples of conjugates and ligands include polyethylene glycol, human serum albumin, or a ligand for a cellular receptor that can mediate cellular uptake.

In some aspects lipids may be employed for delivery and administration of RNA components. In some embodiments, a composition may be a mixture or complex of one or more interfering RNA agents with one or more lipids.

Lipids for delivery and administration of RNA components may include amino acid lipids as described in Quay et al., U.S. Patent Application No. 60/916,131.

Examples of cationic lipids include N-[1-(2,3-dioleoyloxy)propyl]-N,N,N-trimethylammonium chloride (DOTMA); 1,2-bis(oleoyloxy)-3-3-(trimethylammonium)propane (DOTAP), 1,2-bis(dimyrstoyloxy)-3-3-(trimethylammonia)propane (DMTAP); 1,2-dimyristyloxypropyl-3-dimethylhydroxyethylammonium bromide (DMRIE); dimethyldioctadecylammonium bromide (DDAB); 3-(N-(N′,N′-dimethylaminoethane)carbamoyl)cholesterol (DC-Chol); 3β-[N′,N′-diguanidinoethyl-aminoethane)carbamoyl cholesterol (BGTC); 2-(2-(3-(bis(3-aminopropyl)amino)propylamino)acetamido)-N,N-ditetradecylacetamide (RPR209120); pharmaceutically acceptable salts thereof, and mixtures thereof.

Examples of cationic lipids include 1,2-dialkenoyl-sn-glycero-3-ethylphosphocholines (EPCs), such as 1,2-dioleoyl-sn-glycero-3-ethylphosphocholine, 1,2-distearoyl-sn-glycero-3-ethylphosphocholine, 1,2-dipalmitoyl-sn-glycero-3-ethylphosphocholine, pharmaceutically acceptable salts thereof, and mixtures thereof.

Examples of polycationic lipids include tetramethyltetrapalmitoyl spermine (TMTPS), tetramethyltetraoleyl spermine (TMTOS), tetramethlytetralauryl spermine (TMTLS), tetramethyltetramyristyl spermine (TMTMS), tetramethyldioleyl spermine (TMDOS), pharmaceutically acceptable salts thereof, and mixtures thereof.

Examples of polycationic lipids include 2,5-bis(3-aminopropylamino)-N-(2-(dioctadecylamino)-2-oxoethyl)pentanamide (DOGS); 2,5-bis(3-aminopropylamino)-N-(2-(di(Z)-octadeca-9-dienylamino)-2-oxoethyl) pentanamide (DOGS-9-en); 2,5-bis(3-aminopropylamino)-N-(2-(di(9Z,12Z)-octadeca-9,12-dienylamino)-2-oxoethyl)pentanamide (DLinGS); 3-beta-(N⁴-(N¹,N⁸-dicarbobenzoxyspermidine)carbamoyl)cholesterol (GL-67); (9Z,9′Z)-2-(2,5-bis(3-aminopropylamino)pentanamido)propane-1,3-diyl-dioctadec-9-enoate (DOSPER); 2,3-dioleyloxy-N-[2(sperminecarboxamido)ethyl]-N,N-dimethyl-1-propanaminium trifluoro-acetate (DOSPA); pharmaceutically acceptable salts thereof, and mixtures thereof.

Examples of cationic lipids include those shown in Table 2.

TABLE 2
Examples of Cationic Lipids

| Lipid | FA Chains | M.W. | CAS registry # |
|---|---|---|---|
| DS404-28 BGTC | Cholesterol | 642.96 | 182056-06-0 |
| DOSPER | C18:1 | 848.34 | 178532-92-8 |
| GL-67 | Cholesterol | 615.00 | 179075-30-0 |
| RPR209120 | Myristoyl C14 | 695.16 | 433292-13-8 |
| DOGS | C18:0 | 807.37 | 12050-77-7 |
| DOGS (9-en) | C18:1 | 803.34 | |
| DLinGS | C18:2 | 799.31 | |
| DOTMA | C18:1 | 712.57 | 104162-48-3 |

Examples of cationic lipids are described in U.S. Pat. Nos. 4,897,355; 5,279,833; 6,733,777; 6,376,248; 5,736,392; 5,334,761; 5,459,127; 2005/0064595; U.S. Pat. Nos. 5,208,036; 5,264,618; 5,279,833; 5,283,185; 5,753,613; and 5,785,992.

Non-cationic lipids include neutral, zwitterionic, and anionic lipids.

Examples of non-cationic lipids include 1,2-Dilauroyl-sn-glycerol (DLG); 1,2-Dimyristoyl-sn-glycerol (DMG); 1,2-Dipalmitoyl-sn-glycerol (DPG); 1,2-Distearoyl-sn-glycerol (DSG); 1,2-Dilauroyl-sn-glycero-3-phosphatidic acid (sodium salt; DLPA); 1,2-Dimyristoyl-sn-glycero-3-phosphatidic acid (sodium salt; DMPA); 1,2-Dipalmitoyl-sn-glycero-3-phosphatidic acid (sodium salt; DPPA); 1,2-Distearoyl-sn-glycero-3-phosphatidic acid (sodium salt; DSPA); 1,2-Diarachidoyl-sn-glycero-3-phosphocholine (DAPC); 1,2-Dilauroyl-sn-glycero-3-phosphocholine (DLPC); 1,2-Dimyristoyl-sn-glycero-3-phosphocholine (DMPC); 1,2-Dipalmitoyl-sn-glycero-0-ethyl-3-phosphocholine (chloride or triflate; DPePC); 1,2-Dipalmitoyl-sn-glycero-3-phosphocholine (DPPC); 1,2-Distearoyl-sn-glycero-3-phosphocholine (DSPC); 1,2-Dilauroyl-sn-glycero-3-phosphoethanolamine (DLPE); 1,2-Dimyristoyl-sn-glycero-3-phosphoethanolamine (DMPE); 1,2-Dipalmitoyl-sn-glycero-3-phosphoethanolamine (DPPE); 1,2-Distearoyl-sn-glycero-3-phosphoethanolamine (DSPE); 1,2-Dilauroyl-sn-glycero-3-phosphoglycerol (sodium salt; DLPG); 1,2-Dimyristoyl-sn-glycero-3-phosphoglycerol (sodium salt; DMPG); 1,2-Dimyristoyl-sn-glycero-3-phospho-sn-1-glycerol (ammonium salt; DMP-sn-1-G); 1,2-Dipalmitoyl-sn-glycero-3-phosphoglycerol (sodium salt; DPPG); 1,2-Distearoyl-sn-glycero-3-phosphoglycero (sodium salt; DSPG); 1,2-Distearoyl-sn-glycero-3-phospho-sn-1-glycerol (sodium salt; DSP-sn-1-G); 1,2-Dipalmitoyl-sn-glycero-3-phospho-L-serine (sodium salt; DPPS); 1-Palmitoyl-2-linoleoyl-sn-glycero-3-phosphocholine (PLinoPC); 1-Palmitoyl-2-oleoyl-sn-glycero-3-phosphocholine (POPC); 1-Palmitoyl-2-oleoyl-sn-glycero-3-phosphoglycerol (sodium salt; POPG); 1-Palmitoyl-2-oleoyl-sn-glycero-3-phosphoglycerol (sodium salt; POPG); 1-Palmitoyl-2-oleoyl-sn-glycero-3-phosphoglycerol (ammonium salt; POPG); 1-Palmitoyl-2-4o-sn-glycero-3-phosphocholine (P-lyso-PC); 1-Stearoyl-2-lyso-sn-glycero-3-phosphocholine (S-lyso-PC); and mixtures thereof.

Examples of non-cationic lipids include polymeric compounds and polymer-lipid conjugates or polymeric lipids, such as pegylated lipids, including polyethyleneglycols, N-(Carbonyl-methoxypolyethyleneglycol-2000)-1,2-dimyristoyl-sn-glycero-3-phosphoethanolamine (sodium salt; DMPE-MPEG-2000); N-(Carbonyl-methoxypolyethyleneglycol-5000)-1,2-dimyristoyl-sn-glycero-3-phosphoethanolamine (sodium salt; DMPE-MPEG-5000); N-(Carbonyl-methoxypolyethyleneglycol 2000)-1,2-dipalmitoyl-sn-glycero-3-phosphoethanolamine (sodium salt; DPPE-MPEG-2000); N-(Carbonyl-methoxypolyethyleneglycol 5000)-1,2-dipalmitoyl-sn-glycero-3-phosphoethanolamine (sodium salt; DPPE-MPEG-5000); N-(Carbonyl-methoxypolyethyleneglycol 750)-1,2-distearoyl-sn-glycero-3-phosphoethanolamine (sodium salt; DSPE-MPEG-750); N-(Carbonyl-methoxypolyethyleneglycol 2000)-1,2-distearoyl-sn-glycero-3-phosphoethanolamine (sodium salt; DSPE-MPEG-2000); N-(Carbonyl-methoxypolyethyleneglycol 5000)-1,2-distearoyl-sn-glycero-3-phosphoethanolamine (sodium salt; DSPE-MPEG-5000); sodium cholesteryl sulfate (SCS); pharmaceutically acceptable salts thereof, and mixtures thereof.

Examples of non-cationic lipids include dioleoylphosphatidylethanolamine (DOPE), diphytanoylphosphatidylethanolamine (DPhPE), 1,2-Dioleoyl-sn-Glycero-3-Phosphocholine (DOPC), 1,2-Diphytanoyl-sn-Glycero-3-Phosphocholine (DPhPC), cholesterol, and mixtures thereof.

Examples of non-cationic lipids include lipids ranging from C10:0 to C22:6 phosphoethanolamine as shown in Table 3:

TABLE 3
Examples of Non-cationic Lipids

| Name | FA chains | M.W. | CAS Registry # |
|---|---|---|---|
| DDPE | C10:0 | 523.64 | 253685-27-7 |
| DLPE | C12:0 | 579.76 | 59752-57-7 |
| DSPE | C18:0 | 748.08 | 1069-79-0 |
| DOPE | C18:1 | 744.05 | 4004-05-1 |
| DLinPE | C18:2 | 740.01 | 520707-71-5 |
| DLenPE | C18:3 | 735.98 | 534813-40-6 |
| DARAPE | C20:4 | 788.06 | 55634-86-6 |
| DDHAPE | C22:6 | 836.10 | 123284-81-1 |
| DPhPE | 16:0[(CH3)4] | 804.19 | 201036-16-0 |

Examples of anionic lipids include phosphatidylserine, phosphatidic acid, phosphatidylcholine, platelet-activation factor (PAF), phosphatidylethanolamine, phosphatidyl-DL-glycerol, phosphatidylinositol, phosphatidylinositol (pi(4)p, pi(4,5)p2), cardiolipin (sodium salt), lysophosphatides, hydrogenated phospholipids, sphingoplipids, gangliosides, phytosphingosine, sphinganines, pharmaceutically acceptable salts thereof, and mixtures thereof.

In some embodiments, an interfering RNA with a delivery peptide can be combined with a cationic lipid or transfection material such as LIPOFECTAMINE (Invitrogen).

### Substituting and Modifying dsRNA Molecules

The introduction of substituted and modified nucleotides into dicer substrate RNA molecules provides a powerful tool in overcoming potential limitations of in vivo stability and bioavailability inherent to native RNA molecules (i.e., having standard nucleotides) that are exogenously delivered. For example, the use of modified nucleotides in dicer substrate RNA molecules can enable a lower dose of a particular nucleic acid molecule for a given therapeutic effect (e.g., reducing or silencing TNF-α or Influenza specific RNA expression) since modified nucleotides in dicer substrate RNA molecules of this disclosure tend to have a longer half-life in serum. Furthermore, certain substitutions and modifications can improve the bioavailability of dicer substrate RNA by targeting particular cells or tissues or improving cellular uptake of the dicer substrate RNA molecules. Therefore, even if the activity of a dicer substrate RNA molecule of this disclosure is reduced as compared to a native RNA molecule, the overall activity of the substituted or modified dicer substrate RNA molecule can be greater than that of the native RNA molecule due to improved stability or delivery of the molecule. Unlike native unmodified dicer substrate RNA, substituted and modified dicer substrate RNA can also minimize the possibility of activating the interferon response in, e.g., humans.

In certain embodiments, a dicer substrate RNA molecule of this disclosure has at least one uridine, at least three uridines, or each and every uridine (i.e., all uridines) of the first (antisense) strand of the dicer substrate RNA substituted or replaced with 5-methyluridine. In a related embodiment, the dicer substrate RNA molecule or analog thereof of this disclosure has at least one uridine, at least three uridines, or each and every uridine of the second (sense) strand of the dicer substrate RNA substituted or replaced with 5-methyluridine. In still another embodiment, the dicer substrate RNA molecule or analog thereof of this disclosure has at least one uridine, at least three uridines, or each and every uridine of both the first (antisense) and second (sense) strands of the dicer substrate RNA substituted or replaced with 5-methyluridine. In some embodiments, the double-stranded region of a dicer substrate RNA molecule has at least three 5-methyluridines. In certain embodiments, dicer substrate RNA molecules comprise ribonucleotides at about 5% to about 95% of the nucleotide positions in one strand, both strands, or any combination thereof.

In further embodiments, a dicer substrate RNA molecule that decreases expression of a target gene by RNAi according to the instant disclosure further comprises one or more natural or synthetic non-standard nucleoside. In related embodiments, the non-standard nucleoside is one or more deoxyuridine, locked nucleic acid (LNA) molecule (e.g., a 5-methyluridine, LNA), or a universal-binding nucleotide. In certain embodiments, the universal-binding nucleotide can be C-phenyl, C-naphthyl, inosine, azole carboxamide, 1-β-D-ribofuranosyl-4-nitroindole, 1-β-D-ribofuranosyl-5-nitroindole, 1-β-D-ribofuranosyl-6-nitroindole, or 1-β-D-ribofuranosyl-3-nitropyrrole.

Substituted or modified nucleotides present in dicer substrate RNA molecules, preferably in the antisense strand, but also optionally in the sense or both the antisense and sense strands, comprise modified or substituted nucleotides according to this disclosure having properties or characteristics similar to natural or standard ribonucleotides. For example, this disclosure features dicer substrate RNA molecules including nucleotides having a northern conformation (e.g., northern pseudorotation cycle, see, e.g., Saenger, Principles of Nucleic Acid Structure, Springer-Verlag Ed., 1984). As such, chemically modified nucleotides present in dicer substrate RNA molecules of this disclosure, preferably in the antisense strand, but also optionally in the sense or both the antisense and sense strands, are resistant to nuclease degradation while at the same time maintaining the capacity to mediate RNAi. Exemplary nucleotides having a northern configuration include locked nucleic acid (LNA) nucleotides (e.g., 2′-O,4′-C-methylene-(D-ribofuranosyl) nucleotides); 2′-methoxyethyl (MOE) nucleotides; 2′-methyl-thio-ethyl, 2′-deoxy-2′-fluoro nucleotides. 2′-deoxy-2′-chloro nucleotides, 2′-azido nucleotides, 5-methyluridines, or 2′-O-methyl nucleotides. In certain embodiments, the LNA is a 5-methyluridine LNA.

As described herein, the first and second strands of a dicer substrate RNA molecule or analog thereof provided by this disclosure can anneal or hybridize together (i.e., due to complementarity between the strands) to form at least one double-stranded region having a length of about 25 to about 30 base pairs. In other embodiments, the dicer substrate RNA has at least one double-stranded region ranging in length from about 26 to about 40 base pairs or about 27 to about 30 base pairs or about 30 to about 35 base pairs. In other embodiments, the two or more strands of a dicer substrate RNA molecule of this disclosure may optionally be covalently linked together by nucleotide or non-nucleotide linker molecules.

In certain embodiments, the dicer substrate RNA molecule or analog thereof comprises an overhang of one to four nucleotides on one or both 3′-ends of the dicer substrate RNA, such as an overhang comprising a deoxyribonucleotide or two deoxyribonucleotides (e.g., thymidine, adenine). In some embodiments, dicer substrate RNA molecules or analogs thereof have a blunt end at one or both ends of the dicer substrate RNA. In certain embodiments, the 5′-end of the first or second strand is phosphorylated. In any of the embodiments of dicer substrate RNA molecules described herein, the 3′-terminal nucleotide overhangs can comprise ribonucleotides or deoxyribonucleotides that are chemically-modified at a nucleic acid sugar, base, or backbone. In any of the embodiments of dicer substrate RNA molecules described herein, the 3′-terminal nucleotide overhangs can comprise one or more universal ribonucleotides. In any of the embodiments of dicer substrate RNA molecules described herein, the 3′-terminal nucleotide overhangs can comprise one or more acyclic nucleotides. In any of the embodiments of dicer substrate RNA molecules described herein, the dicer substrate RNA can further comprise a terminal phosphate group, such as a 5′-phosphate (see Martinez et al., Cell. 110:563-574, 2002; and Schwarz et al., Molec. Cell 10:537-568, 2002) or a 5′,3′-diphosphate.

As set forth herein, the terminal structure of dicer substrate RNAs of this disclosure that decrease expression of a target gene by, e.g., RNAi, may either have blunt ends or one or more overhangs. In certain embodiments, the overhang may be at the 3′-end or the 5′-end. Furthermore, since the overhanging sequence may have low specificity to a target gene, it is not necessarily complementary (antisense) or identical (sense) to a target gene sequence. In further embodiments, a dicer substrate RNA of this disclosure that decreases expression of a target gene by RNAi may further comprise a low molecular weight structure (e.g., a natural RNA molecule such as a tRNA, rRNA or viral RNA, or an artificial RNA molecule) at, e.g., one or more overhanging portion of the dicer substrate RNA.

In further embodiments, a dicer substrate RNA molecule that decreases expression of a target gene by RNAi according to the instant disclosure further comprises a 2′-sugar substitution, such as 2′-deoxy, 2′-O-methyl, 2′-O-methoxyethyl, 2′-O-2-methoxyethyl, halogen, 2′-fluoro, 2′-O-allyl, or the like, or any combination thereof. In still further embodiments, a dicer substrate RNA molecule that decreases expression of a target gene by RNAi according to the instant disclosure further comprises a terminal cap substituent on one or both ends of the first strand or second strand, such as an alkyl, abasic, deoxy abasic, glyceryl, dinucleotide, acyclic nucleotide, inverted deoxynucleotide moiety, or any combination thereof. In certain embodiments, at least one or two 5′-terminal ribonucleotides of the sense strand within the double-stranded region have a 2′-sugar substitution. In certain other embodiments, at least one or two 5′-terminal ribonucleotides of the antisense strand within the double-stranded region have a 2′-sugar substitution. In certain embodiments, at least one or two 5′-terminal ribonucleotides of the sense strand and the antisense strand within the double-stranded region have a 2′-sugar substitution.

In yet other embodiments, a dicer substrate RNA molecule that decreases expression of a target gene (including an mRNA splice variant thereof) by RNAi according to the instant disclosure further comprises at least one modified internucleoside linkage, such as independently a phosphorothioate, chiral phosphorothioate, phosphorodithioate, phosphotriester, aminoalkylphosphotriester, methyl phosphonate, alkyl phosphonate, 3′-alkylene phosphonate, 5′-alkylene phosphonate, chiral phosphonate, phosphonoacetate, thiophosphonoacetate, phosphinate, phosphoramidate, 3′-amino phosphoramidate, aminoalkylphosphoramidate, thionophosphoramidate, thionoalkylphosphonate, thionoalkylphosphotriester, selenophosphate, boranophosphate linkage, or any combination thereof.

A modified internucleotide linkage, as described herein, can be present in one or more strands of a dicer substrate RNA molecule of this disclosure, e.g., in the sense strand, the antisense strand, both strands, or a plurality of strands. The dicer substrate RNA molecules of this disclosure can comprise one or more modified internucleotide linkages at the 3′-end, the 5′-end, or both of the 3′- and 5′-ends of the sense strand or the antisense strand or both strands. In one embodiment, a dicer substrate RNA molecule capable of decreasing expression of a target gene (including a specific or selected mRNA splice variant thereof) by RNAi has one modified internucleotide linkage at the 3′-end, such as a phosphorothioate linkage. For example, this disclosure provides a dicer substrate RNA molecule capable of decreasing expression of a target gene by RNAi having about 1 to about 8 or more phosphorothioate internucleotide linkages in one dicer substrate RNA strand. In yet another embodiment, this disclosure provides a dicer substrate RNA molecule capable of decreasing expression of a target gene by RNAi having about 1 to about 8 or more phosphorothioate internucleotide linkages in both dicer substrate RNA strands. In other embodiments, an exemplary dicer substrate RNA molecule of this disclosure can comprise from about 1 to about 5 or more consecutive phosphorothioate internucleotide linkages at the 5′-end of the sense strand, the antisense strand, both strands, or a plurality of strands. In another example, an exemplary dicer substrate RNA molecule of this disclosure can comprise one or more pyrimidine phosphorothioate internucleotide linkages in the sense strand, the antisense strand, both strands, or a plurality of strands. In yet another example, an exemplary dsRNA molecule of this disclosure can comprise one or more purine phosphorothioate internucleotide linkages in the sense strand, the antisense strand, both strands or a plurality of strands.

In another aspect of the instant disclosure, there is provided a dicer substrate RNA that decreases expression of a target gene, comprising a first strand that is complementary to a target mRNA and a second strand that is complementary to the first strand, wherein the first and second strands form a double-stranded region of about 25 to about 30 base pairs or about 25 to about 40 base pairs; wherein at least one pyrimidine of the dsRNA is substituted with a pyrimidine nucleoside according to Formula (I) or (II):

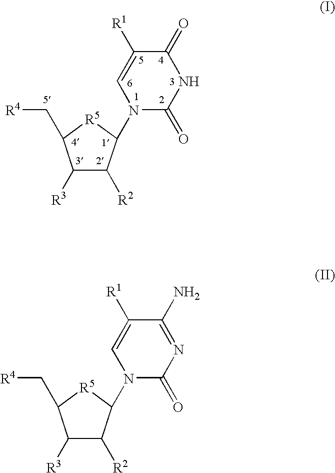

wherein R¹ and R² are each independently a —H, —OH, —OCH₃, —OCH₂OCH₂CH₃, —OCH₂CH₂OCH₃, halogen, substituted or unsubstituted C₁-C₁₀ alkyl, alkoxy, alkoxyalkyl, hydroxyalkyl, carboxyalkyl, alkylsulfonylamino, aminoalkyl, dialkylamino, alkylaminoalkyl, dialkylaminoalkyl, haloalkyl, trifluoromethyl, cycloalkyl, (cycloalkyl)alkyl, substituted or unsubstituted C₂-C₁₀ alkenyl, substituted or unsubstituted —O-allyl, —O—CH₂CH═CH₂, —O—CH═CHCH₃, substituted or unsubstituted C₂-C₁₀ alkynyl, carbamoyl, carbamyl, carboxy, carbonylamino, substituted or unsubstituted aryl, substituted or unsubstituted aralkyl, —NH₂, —NO₂, —C≡N, or heterocyclo group; R³ and R⁴ are each independently a hydroxyl, a protected hydroxyl, or an internucleoside linking group; and R⁵ is O or S. In certain embodiments, at least one nucleoside is according to Formula (I) in which R¹ is methyl and R² is —OH. In other embodiments, the internucleoside linking group covalently links from about 5 to about 40 nucleosides.

In certain embodiments, the first and second strands of a dicer substrate RNA, which decreases expression of a target gene by RNAi and has at least one pyrimidine substituted with a pyrimidine nucleoside according to Formula (I) or (II), can anneal or hybridize together (i.e., due to complementarity between the strands) to form at least one double-stranded region having a length or a combined length of about 25 to about 30 base pairs or about 25 to about 40 base pairs. In some embodiments, the dicer substrate RNA has at least one double-stranded region ranging in length from about 25 base pairs to about 30 base pairs. In other embodiments, the dicer substrate RNA has at least one double-stranded region ranging in length from about 26 to about 40 base pairs or about 27 to about 30 base pairs or about 30 to about 35 base pairs. In certain embodiments, the dicer substrate RNA molecule or analog thereof has an overhang of one to four nucleotides on one or both 3′-ends, such as an overhang comprising a deoxyribonucleotide or two deoxyribonucleotides (e.g., thymidine). In some embodiments, dicer substrate RNA molecule or analog thereof has a blunt end at one or both ends of the dicer substrate RNA. In certain embodiments, the 5′-end of the first or second strand is phosphorylated.

In certain embodiments, at least one R¹ is a C₁-C₅ alkyl, such as methyl or ethyl. Within other exemplary embodiments of this disclosure, compounds of Formula (I) are a 5-alkyluridine (i.e., R¹ is alkyl, R² is —OH, and R³, R⁴, and R⁵ are as defined herein) or compounds of Formula (II) are a 5-alkylcytidine (i.e., R¹ is alkyl, R² is —OH, and R³, R⁴, and R⁵ are as defined herein). In related embodiments, the 5-alkyluridine is a 5-methyluridine (also referred to as ribothymidine or T^{r}—i.e., R¹ is methyl and R² is —OH), and the 5-alkylcytidine is a 5-methylcytidine. In other embodiments, at least one, at least three, or all uridines of the first strand of the dicer substrate RNA are replaced with 5-methyluridine, or at least one, at least three, or all uridines of the second strand of the dicer substrate RNA are replaced with 5-methyluridine, or any combination thereof (e.g., such changes are made on both strands). In certain embodiments, at least one pyrimidine nucleoside of Formula (I) or Formula (II) has an R⁵ that is S.

In further embodiments, at least one pyrimidine nucleoside of the dicer substrate RNA is a locked nucleic acid (LNA) in the form of a bicyclic sugar, wherein R² is oxygen, and the 2′-O and 4′-C form an oxymethylene bridge on the same ribose ring. In a related embodiment, the LNA is having a base substitution, such as a 5-methyluridine LNA. In other embodiments, at least one, at least three, or all uridines of the first strand of the dicer substrate RNA are replaced with 5-methyluridine or 5-methyluridine LNA, or at least one, at least three, or all uridines of the second strand of the dicer substrate RNA are replaced with 5-methyluridine, 5-methyluridine LNA, or any combination thereof (e.g., such changes are made on both strands, or some substitutions include 5-methyluridine only, 5-methyluridine LNA only, or one or more 5-methyluridine with one or more 5-methyluridine LNA).

In further embodiments, a ribose of the pyrimidine nucleoside or the internucleoside linkage can be optionally modified. For example, compounds of Formula (I) or (II) are provided wherein R² is alkoxy, such as a 2′-O-methyl substitution (e.g., which may be in addition to a 5-alkyluridine or a 5-alkylcytidine, respectively). In certain embodiments, R² is selected from 2′-O-(C₁-C₅) alkyl, 2′-O-methyl, 2′-OCH₂OCH₂CH₃, 2′-OCH₂CH₂OCH₃, 2′-O-allyl, or fluoro. In further embodiments, one or more of the pyrimidine nucleosides are according to Formula (I) in which R¹ is methyl and R² is a 2′-O-(C₁-C₅) alkyl (e.g., 2′-O-methyl). In other embodiments, one or more, or at least two, pyrimidine nucleosides according to Formula (I) or (II) have an R² that is not —H or —OH and is incorporated at a 3′-end or 5′-end.

In further embodiments, a dicer substrate RNA molecule or analog thereof comprising a pyrimidine nucleoside according to Formula (I) or Formula (II) in which R² is not —H or —OH and an overhang, further comprises at least two of pyrimidine nucleosides that are incorporated either at a 3′-end or a 5′-end or both of one strand or two strands within the double-stranded region of the dicer substrate RNA molecule. In a related embodiment, at least one of the at least two pyrimidine nucleosides in which R² is not —H or —OH is located at a 3′-end or a 5′-end within the double-stranded region of at least one strand of the dicer substrate RNA molecule, and wherein at least one of the at least two pyrimidine nucleosides in which R² is not —H or —OH is located internally within a strand of the dicer substrate RNA molecule. In still further embodiments, a dicer substrate RNA molecule or analog thereof that has an overhang has a first of the two or more pyrimidine nucleosides in which R² is not —H or —OH that is incorporated at a 5′-end within the double-stranded region of the sense strand of the dicer substrate RNA molecule and a second of the two or more pyrimidine nucleosides is incorporated at a 5′-end within the double-stranded region of the antisense strand of the dicer substrate RNA molecule.

In yet other embodiments, a dicer substrate RNA molecule or analog thereof of Formula (I) or (II) according to the instant disclosure that has an overhang comprises four or more independent pyrimidine nucleosides or four or more independent pyrimidine nucleosides in which R² is not —H or —OH, wherein (a) a first pyrimidine nucleoside is incorporated into a 3′-end within the double-stranded region of the sense (second) strand of the dicer substrate RNA, (b) a second pyrimidine nucleoside is incorporated into a 5′-end within the double-stranded region of the sense (second) strand, (c) a third pyrimidine nucleoside is incorporated into a 3′-end within the double-stranded region of the antisense (first) strand of the dicer substrate RNA, and (d) a fourth pyrimidine nucleoside is incorporated into a 5′-end within the double-stranded region of the antisense (first) strand.

In further embodiments, a dicer substrate RNA molecule or analog thereof comprising a pyrimidine nucleoside according to Formula (I) or Formula (II) in which R² is not —H or —OH and is blunt-ended, further comprises at least two of pyrimidine nucleosides that are incorporated either at a 3′-end or a 5′-end or both of one strand or two strands of the dicer substrate RNA molecule. In a related embodiment, at least one of the at least two pyrimidine nucleosides in which R² is not —H or —OH is located at a 3′-end or a 5′-end of at least one strand of the dicer substrate RNA molecule, and wherein at least one of the at least two pyrimidine nucleosides in which R² is not —H or —OH is located internally within a strand of the dicer substrate RNA molecule. In still further embodiments, a dicer substrate RNA molecule or analog thereof that is blunt-ended has a first of the two or more pyrimidine nucleosides in which R² is not —H or —OH that is incorporated at a 5′-end of the sense strand of the dicer substrate RNA molecule and a second of the two or more pyrimidine nucleosides is incorporated at a 5′-end of the antisense strand of the dicer substrate RNA molecule.

In yet other embodiments, a dicer substrate RNA molecule comprising a pyrimidine nucleoside according to Formula (I) or Formula (II) and that is blunt-ended comprises four or more independent pyrimidine nucleosides or four or more independent pyrimidine nucleosides in which R² is not —H or —OH, wherein (a) a first pyrimidine nucleoside is incorporated into a 3′-end within the double-stranded region of the sense (second) strand of the dicer substrate RNA, (b) a second pyrimidine nucleoside is incorporated into a 5′-end within the double-stranded region of the sense (second) strand, (c) a third pyrimidine nucleoside is incorporated into a 3′-end within the double-stranded region of the antisense (first) strand of the dicer substrate RNA, and (d) a fourth pyrimidine nucleoside is incorporated into a 5′-end within the double-stranded region of the antisense (first) strand.

In still further embodiments, a dicer substrate RNA molecule or analog thereof of Formula (I) or Formula (II) according to the instant disclosure further comprises a terminal cap substituent on one or both ends of the first strand or second strand, such as an alkyl, abasic, deoxy abasic, glyceryl, dinucleotide, acyclic nucleotide, inverted deoxynucleotide moiety, or any combination thereof. In further embodiments, one or more internucleoside linkage can be optionally modified. For example, a dicer substrate RNA molecule or analog thereof of Formula (I) or Formula (II) according to the instant disclosure wherein at least one internucleoside linkage is modified to a phosphorothioate, chiral phosphorothioate, phosphorodithioate, phosphotriester, aminoalkylphosphotriester, methyl phosphonate, alkyl phosphonate, 3′-alkylene phosphonate, 5′-alkylene phosphonate, chiral phosphonate, phosphonoacetate, thiophosphonoacetate, phosphinate, phosphoramidate, 3′-amino phosphoramidate, aminoalkylphosphoramidate, thionophosphoramidate, thionoalkylphosphonate, thionoalkylphosphotriester, selenophosphate, boranophosphate linkage, or any combination thereof.

In still another embodiment, a dicer substrate RNA molecule that decreases expression of a target gene by RNAi, comprising a first strand that is complementary to a target mRNA and a second strand that is complementary to the first strand, wherein the first and second strands form a non-overlapping double-stranded region of about 25 to about 30 base pairs or about 25 to about 40 base pairs. Any of the substitutions or modifications described herein are contemplated within this embodiment as well.

In another exemplary of this disclosure, the dicer substrate RNAs comprise at least two or more substituted pyrimidine nucleosides can each be independently selected wherein R¹ comprises any chemical modification or substitution as contemplated herein, e.g., an alkyl (e.g., methyl), halogen, hydroxy, alkoxy, nitro, amino, trifluoromethyl, cycloalkyl, (cycloalkyl)alkyl, alkanoyl, alkanoyloxy, aryl, aroyl, aralkyl, nitrile, dialkylamino, alkenyl, alkynyl, hydroxyalkyl, aminoalkyl, alkylaminoalkyl, dialkylaminoalkyl, haloalkyl, carboxyalkyl, alkoxyalkyl, carboxy, carbonyl, alkanoylamino, carbamoyl, carbonylamino, alkylsulfonylamino, or heterocyclo group. When two or more modified ribonucleotides are present, each modified ribonucleotide can be independently modified to have the same, or different, modification or substitution at R¹ or R².

In other detailed embodiments, one or more substituted pyrimidine nucleosides according to Formula (I) or Formula (II) can be located at any ribonucleotide position, or any combination of ribonucleotide positions, on either or both of the sense and antisense strands of a dicer substrate RNA molecule of this disclosure, including at one or more multiple terminal positions as noted above, or at any one or combination of multiple non-terminal (“internal”) positions. In this regard, each of the sense and antisense strands can incorporate about 1 to about 6 or more of the substituted pyrimidine nucleosides.

In certain embodiments, when two or more substituted pyrimidine nucleosides are incorporated within a dicer substrate RNA of this disclosure, at least one of the substituted pyrimidine nucleosides will be at a 3′- or 5′-end of one or both strands, and in certain embodiments at least one of the substituted pyrimidine nucleosides will be at a 5′-end of one or both strands. In other embodiments, the substituted pyrimidine nucleosides are located at a position corresponding to a position of a pyrimidine in an unmodified dicer substrate RNA that is constructed as a homologous sequence for targeting a cognate mRNA, as described herein.

In addition, the terminal structure of the dicer substrate RNAs of this disclosure may have a stem-loop structure in which ends of one side of the dicer substrate RNA molecule are connected by a linker nucleic acid, e.g., a linker RNA. The length of the double-stranded region (stem-loop portion) can be, e.g., 25 to 49 bp, often 25 to 35 bp, and more commonly about 25 to 30 bp long. Alternatively, the length of the double-stranded region that is a final transcription product of dicer substrate RNAs to be expressed in a target cell may be, e.g., approximately 25 to 49 bp, 25 to 35 bp, or about 25 to 30 bp long. When linker segments are employed, there is no particular limitation in the length of the linker as long as it does not hinder pairing of the stem portion. For example, for stable pairing of the stem portion and suppression of recombination between DNAs coding for this portion, the linker portion may have a clover-leaf tRNA structure. Even if the linker has a length that would hinder pairing of the stem portion, it is possible, e.g., to construct the linker portion to include introns so that the introns are excised during processing of a precursor RNA into mature RNA, thereby allowing pairing of the stem portion. In the case of a stem-loop dicer substrate RNA, either end (head or tail) of RNA with no loop structure may have a low molecular weight RNA. As described above, these low molecular weight RNAs may include a natural RNA molecule, such as tRNA, rRNA or viral RNA, or an artificial RNA molecule.

A dicer substrate RNA molecule may be comprised of a circular nucleic acid molecule, wherein the dicer substrate RNA is about 38 to about 70 nucleotides in length having from about 25 to about 30 (e.g., about 26 to about 31) base pairs wherein the circular oligonucleotide forms a dumbbell shaped structure having about 25 base pairs and 2 loops. In certain embodiments, a circular dicer substrate RNA molecule contains two loop motifs, wherein one or both loop portions of the dicer substrate RNA molecule is biodegradable. For example, a circular dicer substrate RNA molecule of this disclosure is designed such that degradation of the loop portions of the dsRNA molecule in vivo can generate a double-stranded dicer substrate RNA molecule with 3′-terminal overhangs, such as 3′-terminal nucleotide overhangs comprising from about 1 to about 4 (unpaired) nucleotides.

Substituting pyrimidine nucleosides into a dicer substrate RNA according to this disclosure will often increase resistance to enzymatic degradation, such as exonucleolytic degradation, including 5′-exonucleolytic or 3′-exonucleolytic degradation. As such, the dicer substrate RNAs described herein will exhibit significant resistance to enzymatic degradation compared to a corresponding dicer substrate RNA having standard nucleotides, and will thereby possess greater stability, increased half-life, and greater bioavailability in physiological environments (e.g., when introduced into a eukaryotic target cell). In addition to increasing resistance of the substituted or modified dicer substrate RNAs to exonucleolytic degradation, the incorporation of one or more pyrimidine nucleosides according to Formula (I) or (II) will render dicer substrate RNAs more resistant to other enzymatic or chemical degradation processes, and thus more stable and bioavailable than otherwise identical dicer substrate RNAs that do not include the substitutions or modifications. In related aspects of this disclosure, dicer substrate RNA substitutions or modifications described herein will often improve stability of a modified dicer substrate RNA for use within research, diagnostic and treatment methods wherein the modified dicer substrate RNA is contacted with a biological sample, e.g., a mammalian cell, intracellular compartment, serum or other extra cellular fluid, tissue, or other in vitro or in vivo physiological compartment or environment. In one embodiment, diagnosis is performed on an isolated biological sample. In another embodiment, the diagnostic method is performed in vitro. In a further embodiment, the diagnostic method is not performed (directly) on a human or animal body.

In addition to increasing stability of substituted or modified dicer substrate RNAs, incorporation of one or more pyrimidine nucleosides according to Formula (I) or (II) in a dicer substrate RNA designed for gene silencing will yield additional desired functional results, including increasing a melting point of a substituted or modified dicer substrate RNA compared to a corresponding, unmodified dicer substrate RNA. By thus increasing a dicer substrate RNA melting point, the subject substitutions or modifications will often block or reduce the occurrence or extent of partial dehybridization of the substituted or modified dicer substrate RNA (that would ordinarily occur and render the unmodified dicer substrate RNA more vulnerable to degradation by certain exonucleases), thereby increasing the stability of the substituted or modified dicer substrate RNA.

In another aspect of this disclosure, substitutions or modifications of dicer substrate RNAs described herein will reduce “off-target effects” of the substituted or modified dicer substrate RNA molecules when they are contacted with a biological sample (e.g., when introduced into a target eukaryotic cell having specific, and non-specific mRNA species present as potential specific and non-specific targets). In related embodiments, substituted or modified dicer substrate RNAs according to this disclosure are employed in methods of gene silencing, wherein the substituted or modified dicer substrate RNAs exhibit reduced or eliminated off target effects compared to a corresponding, unmodified dicer substrate RNA, e.g., as determined by non-specific activation of genes in addition to a target (i.e., homologous or cognate) gene in a cell or other biological sample to which the modified dicer substrate RNA is exposed under conditions that allow for gene silencing activity to be detected.

In yet another aspect of this disclosure, the dicer substrate RNA substitutions or modifications described herein will reduce interferon activation by the dicer substrate RNA molecule when the dicer substrate RNA is contacted with a biological sample, e.g., when introduced into a eukaryotic cell.

In further embodiments, dicer substrate RNAs of this disclosure can comprise one or more sense (second) strand that is homologous or corresponds to a sequence of a target gene and an antisense (first) strand that is complementary to the sense strand and a sequence of the target gene. In exemplary embodiments, at least one strand of the dicer substrate RNA incorporates one or more pyrimidines substituted according to Formula (I) or (II) (e.g., wherein the pyrimidine is replaced by more than one 5-methyluridine or the ribose is modified to incorporate a 2′-O-methyl substitution or any combination thereof). These and other multiple substitutions or modifications according to Formula (I) or Formula (II) can be introduced into one or more pyrimidines, or into any combination and up to all pyrimidines present in one or both strands of a dicer substrate RNA.

Within certain aspects, the present disclosure provides dicer substrate RNA that decreases expression of a target gene by RNAi, and compositions comprising one or more dicer substrate RNA, wherein at least one dicer substrate RNA comprises one or more universal-binding nucleotide(s) in the first, second or third position in the anti-codon of the antisense strand of the dicer substrate RNA duplex and wherein the dicer substrate RNA is capable of specifically binding to a target sequence, such as an RNA expressed by a target cell. In cases wherein the sequence of a target RNA includes one or more single nucleotide substitutions, dicer substrate RNA comprising a universal-binding nucleotide retains its capacity to specifically bind a target target RNA, thereby mediating gene silencing and, as a consequence, overcoming escape of the target target from dsRNA-mediated gene silencing. Non-limiting examples of universal-binding nucleotides that may be suitably employed in the compositions and methods disclosed herein include inosine, 1-β-D-ribofuranosyl-5-nitroindole, and 1-β-D-ribofuranosyl-3-nitropyrrole. For the purpose of the present disclosure, a universal-binding nucleotide is a nucleotide that can form a hydrogen bonded nucleotide pair with more than one nucleotide type.

Non-limiting examples for the above compositions includes modifying the anti-codons for tyrosine (AUA) or phenylalanine (AAA or GAA), cysteine (ACA or GCA), histidine (AUG or GUG), asparagine (AUU or GUU), isoleucine (UAU) and aspartate (AUC or GUC) within the anti-codon of the antisense strand of the dsRNA molecule.

For example, within certain embodiments, the isoleucine anti-codon UAU, for which AUA is the cognate codon, may be modified such that the third-position uridine (U) nucleotide is substituted with the universal-binding nucleotide inosine (I) to create the anti-codon UAI. Inosine is an exemplary universal-binding nucleotide that can nucleotide-pair with an adenosine (A), uridine (U), and cytidine (C) nucleotide, but not guanosine (G). This modified anti-codon UAI increases the specific-binding capacity of the dicer substrate RNA molecule and thus permits the dicer substrate RNA to pair with mRNAs having any one of AUA, UUA, and CUA in the corresponding position of the coding strand thereby expanding the number of available RNA degradation targets to which the dicer substrate RNA may specifically bind.

Alternatively, the anti-codon AUA may also or alternatively be modified by substituting a universal-binding nucleotide in the third or second position of the anti-codon such that the anti-codon(s) represented by UAI (third position substitution) or UIU (second position substitution) to generate dicer substrate RNA that are capable of specifically binding to AUA, CUA and UUA and AAA, ACA and AUA.

In certain aspects, dicer substrate RNA disclosed herein can include from about 1 universal-binding nucleotide and about 10 universal-binding nucleotides. Within certain aspects, the presently disclosed dicer substrate RNA may comprise a sense strand that is homologous to a sequence of a target gene and an antisense strand that is complementary to the sense strand, with the proviso that at least one nucleotide of the antisense strand of the otherwise complementary dicer substrate RNA duplex is replaced by one or more universal-binding nucleotide.

By way of background, within the silencing complex, the dicer substrate RNA molecule is positioned so that a target RNA can interact with it. The RISC will encounter thousands of different RNAs that are in a typical cell at any given moment. But, the dicer substrate RNA loaded in RISC will adhere well to a target RNA that has close complementarity with the antisense of the dicer substrate RNA molecule. So, unlike an interferon response to a viral infection, the silencing complex is highly selective in choosing a target RNA. RISC cleaves the captured target RNA strand in two and releases the two pieces of the RNA (now rendered incapable of directing protein synthesis) and moves on. RISC itself stays intact and is capable of finding and cleaving additional target RNA molecules.

It will be understood that, regardless of the position at which the one or more universal-binding nucleotide is substituted, the dicer substrate RNA molecule is capable of binding to a target gene and one or more variant(s) thereof thereby facilitating the degradation of the target gene or variant thereof via a RISC complex. Thus, the dicer substrate RNA of the present disclosure are suitable for introduction into cells to mediate targeted post-transcriptional gene silencing of a target gene or variants thereof.

### Definitions and Terms

As used herein, the term “short interfering nucleic acid,” “siNA,” “short interfering RNA,” “siRNA,” “short interfering nucleic acid molecule,” “short interfering oligonucleotide molecule” or “chemically-modified short interfering nucleic acid molecule” refers to any nucleic acid molecule capable of inhibiting or down regulating gene expression or viral replication, e.g., by mediating RNA interference “RNAi” or gene silencing in a sequence-specific manner. Within exemplary embodiments, the siNA is a double-stranded polynucleotide molecule comprising self-complementary sense and antisense regions, wherein the antisense region comprises a nucleotide sequence that is complementary to a nucleotide sequence in a target nucleic acid molecule for down regulating expression, or a portion thereof, and the sense region comprises a nucleotide sequence corresponding to (i.e., which is substantially identical in sequence to) the target nucleic acid sequence or portion thereof.

“siNA” means a small interfering nucleic acid, e.g., an siRNA, that is a short-length double-stranded nucleic acid (or optionally a longer precursor thereof), and which is not unacceptably toxic in target cells. The length of useful siNAs will in certain embodiments be optimized at a length of approximately 21 to 23 bp long. However, there is no particular limitation in the length of useful siNAs, including siRNAs. For example, siNAs can initially be presented to cells in a precursor form that is substantially different than a final or processed form of the siNA that will exist and exert gene silencing activity upon delivery, or after delivery, to the target cell. Precursor forms of siNAs may, e.g., include precursor RNA components that may be processed to yield an RNAi-inducing entity that is active within the cell to mediate gene silencing. Thus, in certain embodiments, useful siNAs will have a precursor length, e.g., of approximately 100-200 base pairs, 50-100 base pairs, or less than about 50 base pairs, which will yield an active, processed siNA within the target cell. In other embodiments, a useful siNA or siNA precursor will be approximately 10 to 49 bp, 15 to 35 bp, or about 21 to 30 bp in length.

In certain embodiments of this disclosure, as noted above, delivery peptides are used to facilitate delivery of larger nucleic acid molecules than conventional siNAs, including large nucleic acid precursors of siNAs. For example, the methods and compositions herein may be employed for enhancing delivery of larger nucleic acids that represent “precursors” to desired siNAs, wherein the precursor amino acids may be cleaved or otherwise processed before, during or after delivery to a target cell to form an active siNA for modulating gene expression within the target cell. For example, an siNA precursor polynucleotide may be selected as a circular, single-stranded polynucleotide, having two or more loop structures and a stem comprising self-complementary sense and antisense regions, wherein the antisense region comprises a nucleotide sequence that is complementary to a nucleotide sequence in a target nucleic acid molecule or a portion thereof, and the sense region having nucleotide sequence corresponding to the target nucleic acid sequence or a portion thereof, and wherein the circular polynucleotide can be processed either in vivo or in vitro to generate an active siNA molecule capable of mediating RNAi.

In mammalian cells, dsRNAs longer than 30 base pairs can activate the dsRNA-dependent kinase PKR and 2′-5′-oligoadenylate synthetase, normally induced by interferon. The activated PKR inhibits general translation by phosphorylation of the translation factor eukaryotic initiation factor 2α (eIF2α), while 2′-5′-oligoadenylate synthetase causes nonspecific mRNA degradation via activation of RNase L. By virtue of their small size (referring particularly to non-precursor forms), usually less than 30 base pairs, and most commonly from about 17-19, 19-21, or 21-23 base pairs, the siNAs of the present disclosure avoid activation of the interferon response.

In contrast to the nonspecific effect of long dsRNA, siRNA can mediate selective gene silencing in the mammalian system. Hairpin RNAs, with a short loop and 19 to 27 base pairs in the stem, also selectively silence expression of genes that are homologous to the sequence in the double-stranded stem. Mammalian cells can convert short hairpin RNA into siRNA to mediate selective gene silencing.

RISC mediates cleavage of single stranded RNA having sequence complementary to the antisense strand of the siRNA duplex. Cleavage of the target RNA takes place in the middle of the region complementary to the antisense strand of the siRNA duplex. Studies have shown that 21 nucleotide siRNA duplexes are most active when containing a two nucleotide 3′-overhang. Furthermore, complete substitution of one or both siRNA strands with 2′-deoxy (2′-H) or 2′-O-methyl nucleotides abolishes RNAi activity, whereas substitution of the 3′-terminal siRNA overhang nucleotides with deoxy nucleotides (2′-H) has been reported to be tolerated.

In some embodiments of this disclosure, the dsRNA has a 5′ overhang of 2 or more bp, or a 3′ overhang of 2 or more bp, where the overhang may be on either the sense or antisense strand. In some embodiments, the dsRNA has no overhang. In some embodiments, the dsRNA has a length of 27 bp to 29 bp. In some embodiments, the dsRNA molecule contains a sense RNA strand and an antisense RNA strand, and a peptide is conjugated to the 5′ end of the antisense strand.

Alternatively, the siNAs can be delivered as single or multiple transcription products expressed by a polynucleotide vector encoding the single or multiple siNAs and directing their expression within target cells. In these embodiments the double-stranded portion of a final transcription product of the siRNAs to be expressed within the target cell can be, e.g., 15 to 49 bp, 15 to 35 bp, or about 21 to 30 bp long. Within exemplary embodiments, double-stranded portions of siNAs, in which two strands pair up, are not limited to completely paired nucleotide segments, and may contain nonpairing portions due to mismatch (the corresponding nucleotides are not complementary), bulge (lacking in the corresponding complementary nucleotide on one strand), overhang, and the like. Nonpairing portions can be contained to the extent that they do not interfere with siNA formation. In more detailed embodiments, a “bulge” may comprise 1 to 2 nonpairing nucleotides, and the double-stranded region of siNAs in which two strands pair up may contain from about 1 to 7, or about 1 to 5 bulges. In addition, “mismatch” portions contained in the double-stranded region of siNAs may be present in numbers from about 1 to 7, or about 1 to 5. Most often in the case of mismatches, one of the nucleotides is guanine, and the other is uracil. Such mismatching may be attributable, e.g., to a mutation from C to T, G to A, or mixtures thereof, in a corresponding DNA coding for sense RNA, but other cause are also contemplated. Furthermore, the double-stranded region of siNAs in which two strands pair up may contain both bulge and mismatched portions in the approximate numerical ranges specified.

The terminal structure of siNAs may be either blunt or cohesive (overhanging) as long as the siNA retains its activity to silence expression of target genes. The cohesive (overhanging) end structure is not limited only to the 3′ overhang as reported by others. On the contrary, the 5′ overhanging structure may be included as long as it is capable of inducing a gene silencing effect such as by RNAi. In addition, the number of overhanging nucleotides is not limited to reported limits of 2 or 3 nucleotides, but can be any number as long as the overhang does not impair gene silencing activity of the siNA. For example, overhangs may comprise from about 1 to 8 nucleotides, more often from about 2 to 4 nucleotides.

The length of siNAs having cohesive (overhanging) end structure may be expressed in terms of the paired duplex portion and any overhanging portion at each end. For example, a 25/27-mer siNA duplex with a 2-bp 3′ antisense overhang has a 25-mer sense strand and a 27-mer antisense strand, where the paired portion has a length of 25 bp. Furthermore, since the overhanging sequence may have low specificity to a target gene, it is not necessarily complementary (antisense) or identical (sense) to the target gene sequence. Furthermore, as long as the siNA is able to maintain its gene silencing effect on the target gene, it may contain a low molecular weight structure (e.g., a natural RNA molecule such as tRNA, rRNA or viral RNA, or an artificial RNA molecule), e.g., in the overhanging portion at one end.

In addition, the terminal structure of the siNAs may have a stem-loop structure in which ends of one side of the double-stranded nucleic acid are connected by a linker nucleic acid, e.g., a linker RNA. The length of the double-stranded region (stem-loop portion) can be, e.g., 15 to 49 bp, often 15 to 35 bp, and more commonly about 21 to 30 bp long. Alternatively, the length of the double-stranded region that is a final transcription product of siNAs to be expressed in a target cell may be, e.g., approximately 15 to 49 bp, 15 to 35 bp, or about 21 to 30 bp long. When linker segments are employed, there is no particular limitation in the length of the linker as long as it does not hinder pairing of the stem portion. For example, for stable pairing of the stem portion and suppression of recombination between DNAs coding for this portion, the linker portion may have a clover-leaf tRNA structure. Even if the linker has a length that would hinder pairing of the stem portion, it is possible, e.g., to construct the linker portion to include introns so that the introns are excised during processing of a precursor RNA into mature RNA, thereby allowing pairing of the stem portion. In the case of a stem-loop siRNA, either end (head or tail) of RNA with no loop structure may have a low molecular weight RNA. As described above, these low molecular weight RNAs may include a natural RNA molecule, such as tRNA, rRNA or viral RNA, or an artificial RNA molecule.

The siNA can also comprise a single stranded polynucleotide having nucleotide sequence complementary to nucleotide sequence in a target nucleic acid molecule or a portion thereof (e.g., where such siNA molecule does not require the presence within the siNA molecule of nucleotide sequence corresponding to the target nucleic acid sequence or a portion thereof), wherein the single stranded polynucleotide can further comprise a terminal phosphate group, such as a 5′-phosphate (see, e.g., Martinez, et al., Cell. 110:563-574, 2002, and Schwarz, et al., Molecular Cell 10:537-568, 2002), or 5′,3′-diphosphate.

As used herein, the term siNA molecule is not limited to molecules containing only naturally-occurring RNA or DNA, but also encompasses chemically-modified nucleotides and non-nucleotides.

In certain embodiments, the short interfering nucleic acid molecules lack 2′-hydroxy (2′-OH) containing nucleotides. In certain embodiments short interfering nucleic acids do not require the presence of nucleotides having a 2′-hydroxy group for mediating RNAi and as such, short interfering nucleic acid molecules optionally do not include any ribonucleotides (e.g., nucleotides having a 2′-OH group). Such siNA molecules that do not require the presence of ribonucleotides within the siNA molecule to support RNAi can however have an attached linker or linkers or other attached or associated groups, moieties, or chains containing one or more nucleotides with 2′OH groups. Optionally, siNA molecules can comprise ribonucleotides at about 5, 10, 20, 30, 40, or 50% of the nucleotide positions.

As used herein, the term siNA is meant to be equivalent to other terms used to describe nucleic acid molecules that are capable of mediating sequence specific RNAi, e.g., short interfering RNA (siRNA), double-stranded RNA (dsRNA), micro-RNA (mRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), and others.

In other embodiments, siNA molecules may comprise separate sense and antisense sequences or regions, wherein the sense and antisense regions are covalently linked by nucleotide or non-nucleotide linker molecules, or are alternately non-covalently linked by ionic interactions, hydrogen bonding, van der waals interactions, hydrophobic interactions, and/or stacking interactions.

“Antisense RNA” is an RNA strand having a sequence complementary to a target gene mRNA, and thought to induce RNAi by binding to the target gene mRNA. “Sense RNA” has a sequence complementary to the antisense RNA, and annealed to its complementary antisense RNA forms an siRNA. These antisense and sense RNAs have been conventionally synthesized with an RNA synthesizer.

As used herein, the term “RNAi construct” is a generic term used throughout the specification to include small interfering RNAs (siRNAs), hairpin RNAs, and other RNA species which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can produce siRNAs in vivo. Optionally, the siRNA include single strands or double strands of siRNA.

An siHybrid molecule is a double-stranded nucleic acid that has a similar function to siRNA. Instead of a double-stranded RNA molecule, an siHybrid is comprised of an RNA strand and a DNA strand. Preferably, the RNA strand is the antisense strand as that is the strand that binds to the target mRNA. The siHybrid created by the hybridization of the DNA and RNA strands have a hybridized complementary portion and preferably at least one 3′overhanging end.

siNAs can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure, e.g., wherein the double stranded region is about 19 base pairs). The antisense strand may comprise a nucleotide sequence that is complementary to a nucleotide sequence in a target nucleic acid molecule or a portion thereof, and the sense strand may comprise a nucleotide sequence corresponding to the target nucleic acid sequence or a portion thereof. Alternatively, the siNA can be assembled from a single oligonucleotide, where the self-complementary sense and antisense regions of the siNA are linked by means of a nucleic acid-based or non-nucleic acid-based linker(s).

Within additional embodiments, siNAs for intracellular delivery according to the methods and compositions of this disclosure can be a polynucleotide with a duplex, asymmetric duplex, hairpin or asymmetric hairpin secondary structure, having self-complementary sense and antisense regions, wherein the antisense region comprises a nucleotide sequence that is complementary to a nucleotide sequence in a separate target nucleic acid molecule or a portion thereof, and the sense region comprises a nucleotide sequence corresponding to the target nucleic acid sequence or a portion thereof.

Non-limiting examples of chemical modifications that can be made in an siNA include without limitation phosphorothioate internucleotide linkages, 2′-deoxyribonucleotides, 2′-O-methyl ribonucleotides, 2′-deoxy-2′-fluoro ribonucleotides, “universal base” nucleotides, “acyclic” nucleotides, 5-C-methyl nucleotides, and terminal glyceryl and/or inverted deoxy abasic residue incorporation. These chemical modifications, when used in various siNA constructs, are shown to preserve RNAi activity in cells while at the same time, dramatically increasing the serum stability of these compounds.

In a non-limiting example, the introduction of chemically-modified nucleotides into nucleic acid molecules provides a powerful tool in overcoming potential limitations of in vivo stability and bioavailability inherent to native RNA molecules that are delivered exogenously. For example, the use of chemically-modified nucleic acid molecules can enable a lower dose of a particular nucleic acid molecule for a given therapeutic effect since chemically-modified nucleic acid molecules tend to have a longer half-life in serum. Furthermore, certain chemical modifications can improve the bioavailability of nucleic acid molecules by targeting particular cells or tissues and/or improving cellular uptake of the nucleic acid molecule. Therefore, even if the activity of a chemically-modified nucleic acid molecule is reduced as compared to a native nucleic acid molecule, e.g., when compared to an all-RNA nucleic acid molecule, the overall activity of the modified nucleic acid molecule can be greater than that of the native molecule due to improved stability and/or delivery of the molecule. Unlike native unmodified siNA, chemically-modified siNA can also minimize the possibility of activating interferon activity in humans.

The siNA molecules described herein, the antisense region of an siNA molecule of this disclosure can comprise a phosphorothioate internucleotide linkage at the 3′-end of said antisense region. In any of the embodiments of siNA molecules described herein, the antisense region can comprise about one to about five phosphorothioate internucleotide linkages at the 5′-end of said antisense region. In any of the embodiments of siNA molecules described herein, the 3′-terminal nucleotide overhangs of an siNA molecule of the disclosure can comprise ribonucleotides or deoxyribonucleotides that are chemically-modified at a nucleic acid sugar, base, or backbone. In any of the embodiments of siNA molecules described herein, the 3′-terminal nucleotide overhangs can comprise one or more universal base ribonucleotides. In any of the embodiments of siNA molecules described herein, the 3′-terminal nucleotide overhangs can comprise one or more acyclic nucleotides.

For example, in a non-limiting example, a chemically-modified short interfering nucleic acid (siNA) may have about 1, 2, 3, 4, 5, 6, 7, 8 or more phosphorothioate internucleotide linkages in one siNA strand. In yet another embodiment, a chemically-modified short interfering nucleic acid (siNA) individually may have about 1, 2, 3, 4, 5, 6, 7, 8 or more phosphorothioate internucleotide linkages in both siNA strands. The phosphorothioate internucleotide linkages can be present in one or both oligonucleotide strands of the siNA duplex, e.g., in the sense strand, the antisense strand, or both strands. The siNA molecules of this disclosure can comprise one or more phosphorothioate internucleotide linkages at the 3′-end, the 5′-end, or both of the 3′- and 5′-ends of the sense strand, the antisense strand, or both strands. For example, an exemplary siNA molecule can comprise about 1 to about 5 or more (e.g., about 1, 2, 3, 4, 5, or more) consecutive phosphorothioate internucleotide linkages at the 5′-end of the sense strand, the antisense strand, or both strands. In another non-limiting example, an exemplary siNA molecule of this disclosure can comprise one or more (e.g., about 1, 2, 3, 4, 5, 6, 7, 8, 9, 10, or more) pyrimidine phosphorothioate internucleotide linkages in the sense strand, the antisense strand, or both strands. In yet another non-limiting example, an exemplary siNA molecule of this disclosure can comprise one or more (e.g., about 1, 2, 3, 4, 5, 6, 7, 8, 9, 10, or more) purine phosphorothioate internucleotide linkages in the sense strand, the antisense strand, or both strands.

An siNA molecule may be comprised of a circular nucleic acid molecule, wherein the siNA is about 38 to about 70 (e.g., about 38, 40, 45, 50, 55, 60, 65, or 70) nucleotides in length having about 18 to about 23 (e.g., about 18, 19, 20, 21, 22, or 23) base pairs wherein the circular oligonucleotide forms a dumbbell shaped structure having about 19 base pairs and 2 loops.

A circular siNA molecule contains two loop motifs, wherein one or both loop portions of the siNA molecule is biodegradable. For example, a circular siNA molecule of this disclosure is designed such that degradation of the loop portions of the siNA molecule in vivo can generate a double-stranded siNA molecule with 3′-terminal overhangs, such as 3′-terminal nucleotide overhangs comprising about 2 nucleotides.

Modified nucleotides present in siNA molecules, preferably in the antisense strand of the siNA molecules, but also optionally in the sense and/or both antisense and sense strands, comprise modified nucleotides having properties or characteristics similar to naturally occurring ribonucleotides. For example, this disclosure features siNA molecules including modified nucleotides having a northern conformation (e.g., northern pseudorotation cycle, see, e.g., Saenger, Principles of Nucleic Acid Structure, Springer-Verlag Ed., 1984). As such, chemically modified nucleotides present in the siNA molecules of this disclosure, preferably in the antisense strand of the siNA molecules of this disclosure, but also optionally in the sense and/or both antisense and sense strands, are resistant to nuclease degradation while at the same time maintaining the capacity to mediate RNAi. Non-limiting examples of nucleotides having a northern configuration include locked nucleic acid (LNA) nucleotides (e.g., 2′-O, 4′-C-methylene-(D-ribofuranosyl) nucleotides); 2′-methoxyethoxy (MOE) nucleotides; 2′-methyl-thio-ethyl, 2′-deoxy-2′-fluoro nucleotides. 2′-deoxy-2′-chloro nucleotides, 2′-azido nucleotides, and 2′-O-methyl nucleotides.

The sense strand of a double stranded siNA molecule may have a terminal cap moiety such as an inverted deoxybasic moiety, at the 3′-end, 5′-end, or both 3′ and 5′-ends of the sense strand.

An siNA further may be further comprised of a nucleotide, non-nucleotide, or mixed nucleotide/non-nucleotide linker that joins the sense region of the siNA to the antisense region of the siNA. In one embodiment, a nucleotide linker can be a linker of >2 nucleotides in length, e.g., about 3, 4, 5, 6, 7, 8, 9, or 10 nucleotides in length. In another embodiment, the nucleotide linker can be a nucleic acid aptamer. By “aptamer” or “nucleic acid aptamer” as used herein is meant a nucleic acid molecule that binds specifically to a target molecule wherein the nucleic acid molecule has sequence that comprises a sequence recognized by the target molecule in its natural setting. Alternately, an aptamer can be a nucleic acid molecule that binds to a target molecule where the target molecule does not naturally bind to a nucleic acid. The target molecule can be any molecule of interest. For example, the aptamer can be used to bind to a ligand-binding domain of a protein, thereby preventing interaction of the naturally occurring ligand with the protein. This is a non-limiting example and those in the art will recognize that other embodiments can be readily generated using techniques generally known in the art. (See, e.g., Gold, et al, Annu. Rev. Biochem. 64:763, 1995; Brody and Gold, J. Biotechnol 74:5, 2000; Sun, Curr. Opin. Mol. Ther. 2:100, 2000; Kusser, J. Biotechnol. 74:27, 2000; Hermann and Patel, Science 287:820, 2000; and Jayasena, Clinical Chemistry 45:1628, 1999.

A non-nucleotide linker may be comprised of an abasic nucleotide, polyether, polyamine, polyamide, peptide, carbohydrate, lipid, polyhydrocarbon, or other polymeric compounds (e.g., polyethylene glycols such as those having from 2 to 100 ethylene glycol units). Specific examples include those described by Seela and Kaiser, Nucleic Acids Res. 18:6353, 1990 and Nucleic Acids Res. 15:3113, 1987; Cload and Schepartz, J. Am. Chem. Soc. 113:6324, 1991; Richardson and Schepartz, J. Am. Chem. Soc. 113:5109, 1991; Ma, et al., Nucleic Acids Res. 21:2585, 1993 and Biochemistry 32:1751, 1993; Durand, et al., Nucleic Acids Res. 18:6353, 1990; McCurdy, et al., Nucleosides & Nucleotides 10:287, 1991; Jschke, et al., Tetrahedron Lett. 34:301, 1993; Ono, et al., Biochemistry 30:9914 (1991); Arnold, et al., International Publication No. WO 89/02439; Usman, et al., International Publication No. WO 95/06731; Dudycz, et al., International Publication No. WO 95/11910 and Ferentz and Verdine, J. Am. Chem. Soc. 113:4000, 1991. A “non-nucleotide” further means any group or compound that can be incorporated into a nucleic acid chain in the place of one or more nucleotide units, including either sugar and/or phosphate substitutions, and allows the remaining bases to exhibit their enzymatic activity. The group or compound can be abasic in that it does not contain a commonly recognized nucleotide base, such as adenosine, guanine, cytosine, uracil or thymidine, e.g., at the C1 position of the sugar.

The synthesis of an siNA molecule, which can be chemically-modified, comprises: (a) synthesis of two complementary strands of the siNA molecule; (b) annealing the two complementary strands together under conditions suitable to obtain a double-stranded siNA molecule. In another embodiment, synthesis of the two complementary strands of the siNA molecule is by solid phase oligonucleotide synthesis. In yet another embodiment, synthesis of the two complementary strands of the siNA molecule is by solid phase tandem oligonucleotide synthesis.

Oligonucleotides (e.g., certain modified oligonucleotides or portions of oligonucleotides lacking ribonucleotides) are synthesized using protocols known in the art, e.g., as described in Caruthers, et al., Methods in Enzymology 211:3-19, 1992; Thompson, et al., International PCT Publication No. WO 99/54459; Wincott, et al., Nucleic Acids Res. 23:2677-2684, 1995; Wincott, et al., Methods Mol. Bio. 74:59, 1997; Brennan, et al., Biotechnol Bioeng. 61:33-45, 1998; and Brennan, U.S. Pat. No. 6,001,311. Synthesis of RNA, including certain siNA molecules of this disclosure, follows general procedures as described, e.g., in Usman, et al, J. Am. Chem. Soc. 109:7845, 1987; Scaringe, et al., Nucleic Acids Res. 18:5433, 1990; and Wincott, et al., Nucleic Acids Res. 23:2677-2684, 1995; Wincott, et al., Methods Mol. Bio. 74:59, 1997.

Supplemental or complementary methods for delivery of nucleic acid molecules for use herein are described, e.g., in Akhtar, et al., Trends Cell Bio. 2:139, 1992; Delivery Strategies for Antisense Oligonucleotide Therapeutics, ed. Akhtar, 1995; Maurer, et al., Mol. Membr. Biol. 16:129-140, 1999; Hofland and Huang, Handb. Exp. Pharmacol. 137:165-192, 1999; and Lee, et al., ACS Symp. Ser. 752:184-192, 2000. Sullivan, et al., International PCT Publication No. WO 94/02595, further describes general methods for delivery of enzymatic nucleic acid molecules. These protocols can be utilized to supplement or complement delivery of virtually any nucleic acid molecule contemplated within this disclosure.

Nucleic acid molecules and delivery peptides can be administered to cells by a variety of methods known to those of skill in the art, including, but not restricted to, administration within formulations that comprise the siNA and delivery peptide alone, or that further comprise one or more additional components, such as a pharmaceutically acceptable carrier, diluent, excipient, adjuvant, emulsifier, buffer, stabilizer, preservative, and the like. In certain embodiments, the siNA and/or the delivery peptide can be encapsulated in liposomes, administered by iontophoresis, or incorporated into other vehicles, such as hydrogels, cyclodextrins, biodegradable nanocapsules, bioadhesive microspheres, or proteinaceous vectors (see, e.g., O'Hare and Normand, International PCT Publication No. WO 00/53722). Alternatively, a nucleic acid/peptide/vehicle combination can be locally delivered by direct injection or by use of an infusion pump. Direct injection of the nucleic acid molecules of this disclosure, whether subcutaneous, intramuscular, or intradermal, can take place using standard needle and syringe methodologies, or by needle-free technologies such as those described in Conry et al., Clin. Cancer Res. 5:2330-2337, 1999 and Barry et al., International PCT Publication No. WO 99/31262.

The compositions of the instant disclosure can be effectively employed as pharmaceutical agents. Pharmaceutical agents prevent, modulate the occurrence or severity of, or treat (alleviate one or more symptom(s) to a detectable or measurable extent) of a disease state or other adverse condition in a patient.

Thus within additional embodiments this disclosure provides pharmaceutical compositions and methods featuring the presence or administration of one or more polynucleic acid(s), typically one or more siNAs, combined, complexed, or conjugated with a delivery peptide, optionally formulated with a pharmaceutically-acceptable carrier, such as a diluent, stabilizer, buffer, and the like.

The present disclosure satisfies additional objects and advantages by providing short interfering nucleic acid (siNA) molecules that modulate expression of genes associated with a particular disease state or other adverse condition in a subject. Typically, the siNA will target a gene that is expressed at an elevated level as a causal or contributing factor associated with the subject disease state or adverse condition. In this context, the siNA will effectively downregulate expression of the gene to levels that prevent, alleviate, or reduce the severity or recurrence of one or more associated disease symptoms. Alternatively, for various distinct disease models where expression of the target gene is not necessarily elevated as a consequence or sequel of disease or other adverse condition, down regulation of the target gene will nonetheless result in a therapeutic result by lowering gene expression (i.e., to reduce levels of a selected mRNA and/or protein product of the target gene). Alternatively, siNAs of this disclosure may be targeted to lower expression of one gene, which can result in upregulation of a “downstream” gene whose expression is negatively regulated by a product or activity of the target gene.

Within exemplary embodiments, the compositions and methods of this disclosure are useful as therapeutic tools to regulate expression of tumor necrosis factor-α (TNF-α) to treat or prevent symptoms of rheumatoid arthritis (RA). In this context this disclosure further provides compounds, compositions, and methods useful for modulating expression and activity of TNF-α by RNA interference (RNAi) using small nucleic acid molecules. In more detailed embodiments, this disclosure provides small nucleic acid molecules, such as short interfering nucleic acid (siNA), short interfering RNA (siRNA), double-stranded RNA (dsRNA), micro-RNA (mRNA), and short hairpin RNA (shRNA) molecules, and related methods, that are effective for modulating expression of TNF-α and/or TNF-α genes to prevent or alleviate symptoms of RA in mammalian subjects. Within these and related therapeutic compositions and methods, the use of chemically-modified siNAs will often improve properties of the modified siNAs in comparison to properties of native siNA molecules, e.g., by providing increased resistance to nuclease degradation in vivo, and/or through improved cellular uptake. As can be readily determined according to the disclosure herein, useful siNAs having multiple chemical modifications will retain their RNAi activity. The siNA molecules of this disclosure thus provide useful reagents and methods for a variety of therapeutic, diagnostic, target validation, genomic discovery, genetic engineering, and pharmacogenomic applications.

This siNAs of this disclosure may be administered in any form, e.g., transdermally or by local injection (e.g., local injection at sites of psoriatic plaques to treat psoriasis, or into the joints of patients afflicted with psoriatic arthritis or RA). In some embodiments, this disclosure provides formulations and methods to administer therapeutically effective amounts of siNAs directed against an mRNA of TNF-α, which effectively down-regulate the TNF-α RNA and thereby reduce or prevent one or more TNF-α-associated inflammatory condition(s). Comparable methods and compositions are provided that target expression of one or more different genes associated with a selected disease condition in animal subjects, including any of a large number of genes whose expression is known to be aberrantly increased as a causal or contributing factor associated with the selected disease condition.

The siNA/delivery peptide mixtures of this disclosure can be administered in conjunction with other standard treatments for a targeted disease condition, e.g., in conjunction with therapeutic agents effective against inflammatory diseases, such as RA or psoriasis. Examples of combinatorially useful and effective agents in this context include non-steroidal anti-inflammatory drugs (NSAIDs), methotrexate, gold compounds, D-penicillamine, the antimalarials, sulfasalazine, glucocorticoids, and other TNF-α neutralizing agents such as infliximab and entracept.

Negatively charged polynucleotides of this disclosure (e.g., RNA or DNA) can be administered to a patient by any standard means, with or without stabilizers, buffers, and the like, to form a pharmaceutical composition. When it is desired to use a liposome delivery mechanism, standard protocols for formation of liposomes can be followed. The compositions of the present disclosure may also be formulated and used as tablets, capsules or elixirs for oral administration, suppositories for rectal administration, sterile solutions, suspensions for injectable administration, and the other compositions known in the art.

The present disclosure also includes pharmaceutically acceptable formulations of the compositions described herein. These formulations include salts of the above compounds, e.g., acid addition salts, e.g., salts of hydrochloric, hydrobromic, acetic acid, and benzene sulfonic acid.

A pharmacological composition or formulation refers to a composition or formulation in a form suitable for administration, e.g., systemic administration, into a cell or patient, including, for example, a human. Suitable forms, in part, depend upon the use or the route of entry, e.g., oral, transdermal, or by injection. Such forms should not prevent the composition or formulation from reaching a target cell (i.e., a cell to which the negatively charged nucleic acid is desirable for delivery). For example, pharmacological compositions injected into the blood stream should be soluble. Other factors are known in the art, and include considerations such as toxicity.

By “systemic administration” is meant in vivo systemic absorption or accumulation of drugs in the blood stream followed by distribution throughout the entire body. Administration routes which lead to systemic absorption include, without limitation: intravenous, subcutaneous, intraperitoneal, inhalation, oral, intrapulmonary and intramuscular. Each of these administration routes exposes the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant disclosure can potentially localize the drug, e.g., in certain tissue types, such as the tissues of the reticular endothelial system (RES). A liposome formulation that can facilitate the association of drug with the surface of cells, such as, lymphocytes and macrophages is also useful. This approach may provide enhanced delivery of the drug to target cells by taking advantage of the specificity of macrophage and lymphocyte immune recognition of abnormal cells, such as cancer cells.

By “pharmaceutically acceptable formulation” is meant, a composition or formulation that allows for the effective distribution of the nucleic acid molecules of the instant disclosure in the physical location most suitable for their desired activity. Nonlimiting examples of agents suitable for formulation with the nucleic acid molecules of the instant disclosure include: P-glycoprotein inhibitors (such as Pluronic P85), which can enhance entry of drugs into the CNS (Jolliet-Riant and Tillement, Fundam. Clin. Pharmacol. 13:16-26, 1999); biodegradable polymers, such as poly (DL-lactide-coglycolide) microspheres for sustained release delivery after intracerebral implantation (Emerich, D. F., et al., Cell Transplant 8:47-58, 1999) (Alkermes, Inc. Cambridge, Mass.); and loaded nanoparticles, such as those made of polybutylcyanoacrylate, which can deliver drugs across the blood brain barrier and can alter neuronal uptake mechanisms (Prog. Neuropsychopharmacol Biol. Psychiatry 23:941-949, 1999). Other non-limiting examples of delivery strategies for the nucleic acid molecules of the instant disclosure include material described in Boado, et al., J. Pharm. Sci. 87:1308-1315, 1998; Tyler, et al., FEBS Lett. 421:280-284, 1999; Pardridge, et al, PNAS USA. 92:5592-5596, 1995; Boado, Adv. Drug Delivery Rev. 15:73-107, 1995; Aldrian-Herrada, et al., Nucleic Acids Res. 26:4910-4916, 1998; and Tyler, et al., PNAS USA. 96:7053-7058, 1999.

The present disclosure also includes compositions prepared for storage or administration, which include a pharmaceutically effective amount of the desired compounds in a pharmaceutically acceptable carrier or diluent. Acceptable carriers or diluents for therapeutic use are well known in the pharmaceutical art, and are described, e.g., in Remington's Pharmaceutical Sciences, Mack Publishing Co., A. R. Gennaro Ed., 1985. For example, preservatives, stabilizers, dyes and flavoring agents may be provided. These include sodium benzoate, sorbic acid and esters of p-hydroxybenzoic acid. In addition, antioxidants and suspending agents may be used.

A pharmaceutically effective dose is that dose required to prevent, inhibit the occurrence of, or treat (alleviate a symptom to some extent, preferably all of the symptoms) a disease state. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount from about 0.01 mg/kg and 25 mg/kg body weight/day of active ingredients is administered dependent upon potency of the negatively charged polymer.

Aqueous suspensions contain the active materials in admixture with excipients suitable for the manufacture of aqueous suspensions. Such excipients are suspending agents, e.g., sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, e.g., lecithin, or condensation products of an alkylene oxide with fatty acids, e.g., polyoxyethylene stearate, or condensation products of ethylene oxide with long chain aliphatic alcohols, e.g., heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, e.g., polyethylene sorbitan monooleate. The aqueous suspensions can also contain one or more preservatives, e.g., ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, e.g., arachis oil, olive oil, sesame oil or coconut oil, or in a mineral oil such as liquid paraffin. The oily suspensions can contain a thickening agent, e.g., beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, e.g., sweetening, flavoring and coloring agents, can also be present.

Pharmaceutical compositions of the disclosure can also be in the form of oil-in-water emulsions. The oily phase can be a vegetable oil or a mineral oil or mixtures of these. Suitable emulsifying agents can be naturally-occurring gums, e.g., gum acacia or gum tragacanth, naturally-occurring phosphatides, e.g., soy bean, lecithin, and esters or partial esters derived from fatty acids and hexitol, anhydrides, e.g., sorbitan monooleate, and condensation products of the said partial esters with ethylene oxide, e.g., polyoxyethylene sorbitan monooleate. The emulsions can also contain sweetening and flavoring agents.

The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, e.g., as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono-or diglycerides. In addition, fatty acids such as oleic acid find use in the preparation of injectables.

The siNAs can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

The siNAs can be modified extensively to enhance stability by modification with nuclease resistant groups, e.g., 2′-amino, 2′-C-allyl, 2′-fluoro, 2′-O-methyl, 2′-H. (For a review see Usman and Cedergren, TIBS 17:34, 1992; Usman, et al., Nucleic Acids Symp. Ser. 31:163, 1994). SiNA constructs can be purified by gel electrophoresis using general methods or can be purified by high pressure liquid chromatography and re-suspended in water.

Chemically synthesizing nucleic acid molecules with modifications (base, sugar and/or phosphate) can prevent their degradation by serum ribonucleases, which can increase their potency. See, e.g., Eckstein, et al., International Publication No. WO 92/07065; Perrault, et al., Nature 344:565, 1990; Pieken, et al., Science 253:314, 1991; Usman and Cedergren, Trends in Biochem. Sci. 17:334, 1992; Usman, et al, International Publication No. WO 93/15187; and Rossi, et al., International Publication No. WO 91/03162; Sproat, U.S. Pat. No. 5,334,711; Gold, et al., U.S. Pat. No. 6,300,074. All of the above references describe various chemical modifications that can be made to the base, phosphate and/or sugar moieties of the nucleic acid molecules described herein.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, e.g., 2′-amino, 2′-C-allyl, 2′-fluoro, 2′-O-methyl, 2′-O-allyl, 2′-H, nucleotide base modifications. For a review see Usman and Cedergren, TIBS 17:34, 1992; Usman, et al., Nucleic Acids Symp. Ser. 31:163, 1994; Burgin, et al., Biochemistry 35:14090, 1996. Sugar modification of nucleic acid molecules have been extensively described in the art. See Eckstein, et al., International Publication PCT No. WO 92/07065; Perrault, et al., Nature 344:565-568, 1990; Pieken, et al., Science 253:314-317, 1991; Usman and Cedergren, Trends in Biochem. Sci. 17:334-339, 1992; Usman, et al., International Publication PCT No. WO 93/15187; Sproat, U.S. Pat. No. 5,334,711 and Beigelman, et al., J. Biol. Chem. 270:25702, 1995; Beigelman, et al., International PCT publication No. WO 97/26270; Beigelman, et al., U.S. Pat. No. 5,716,824; Usman, et al., U.S. Pat. No. 5,627,053; Woolf, et al., International PCT Publication No. WO 98/13526; Thompson, et al., Karpeisky, et al., Tetrahedron Lett. 39:1131, 1998; Earnshaw and Gait, Biopolymers (Nucleic Acid Sciences) 48:39-55, 1998; Verma and Eckstein, Annu. Rev. Biochem. 67:99-134, 1998; and Burlina, et al, Bioorg. Med. Chem. 5:1999-2010, 1997. Such publications describe general methods and strategies to determine the location of incorporation of sugar, base and/or phosphate modifications and the like into nucleic acid molecules without modulating catalysis. In view of such teachings, similar modifications can be used as described herein to modify the siNA nucleic acid molecules of the instant disclosure so long as the ability of siNA to promote RNAi in cells is not significantly inhibited.

While chemical modification of oligonucleotide internucleotide linkages with phosphorothioate, phosphorodithioate, and/or 5′-methylphosphonate linkages improves stability, excessive modifications can cause some toxicity or decreased activity. Therefore, when designing nucleic acid molecules, the amount of these internucleotide linkages should be minimized. The reduction in the concentration of these linkages should lower toxicity, resulting in increased efficacy and higher specificity of these molecules.

In one embodiment, this disclosure features modified siNA molecules, with phosphate backbone modifications comprising one or more phosphorothioate, phosphorodithioate, methylphosphonate, phosphotriester, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications, see Hunziker and Leumann, “Nucleic Acid Analogues: Synthesis and Properties, in Modern Synthetic Methods,” VCH, 331-417, 1995, and Mesmaeker, et al, “Novel Backbone Replacements for Oligonucleotides, in Carbohydrate Modifications in Antisense Research,” ACS, 24-39, 1994.

Methods for the delivery of nucleic acid molecules are described in Akhtar, et al., Trends Cell Bio. 2:139, 1992; Delivery Strategies for Antisense Oligonucleotide Therapeutics, ed. Akhtar, 1995; Maurer, et al., Mol. Membr. Biol. 16:129-140, 1999; Hofland and Huang, Handb. Exp. Pharmacol. 137:165-192, 1999; and Lee, et al, ACS Symp. Ser. 752:184-192, 2000. Beigelman, et al., U.S. Pat. No. 6,395,713 and Sullivan, et al., PCT WO 94/02595 further describe the general methods for delivery of nucleic acid molecules. These protocols can be utilized for the delivery of virtually any nucleic acid molecule. Nucleic acid molecules can be administered to cells by a variety of methods known to those of skill in the art, including, but not restricted to, encapsulation in liposomes, by iontophoresis, or by incorporation into other vehicles, such as biodegradable polymers, hydrogels, cyclodextrins (see, e.g., Gonzalez, et al., Bioconjugate Chem. 10:1068-1074, 1999; Wang, et al, International PCT Publication Nos. WO 03/47518 and WO 03/46185), poly(lactic-co-glycolic)ac-id (PLGA) and PLCA microspheres (see, e.g., U.S. Pat. No. 6,447,796 and U.S. Patent Application Publication No. US 2002130430), biodegradable nanocapsules, and bioadhesive microspheres, or by proteinaceous vectors (O'Hare and Normand, International PCT Publication No. WO 00/53722). Alternatively, the nucleic acid/vehicle combination is locally delivered by direct injection or by use of an infusion pump. Direct injection of the nucleic acid molecules of this disclosure, whether subcutaneous, intramuscular, or intradermal, can take place using standard needle and syringe methodologies, or by needle-free technologies such as those described in Conry, et al., Clin. Cancer Res. 5:2330-2337, 1999, and Barry, et al., International PCT Publication No. WO 99/31262. The molecules of the instant disclosure can be used as pharmaceutical agents. Pharmaceutical agents prevent, modulate the occurrence, or treat (alleviate a symptom to some extent, preferably all of the symptoms) of a disease state in a subject.

The nucleic acid compositions and formulations of this disclosure may be administered by various routes, e.g., to effect systemic delivery via intravenous, parenteral, or intraperitoneal routes. In some embodiments, a dsRNA may be delivered intracellularly, e.g., in cells of a target tissue such as lung or liver, or in inflamed tissues. Included within this disclosure are compositions and methods for delivery of an siRNA agent by removing cells of a subject, delivering an siRNA agent to the removed cells, and reintroducing the cells into a subject. In some embodiments, this disclosure provides a method for delivery of siRNA in vivo. A nucleic acid composition may be administered intravenously, subcutaneously, or intraperitoneally to a subject. In some embodiments, this disclosure provides methods for in vivo delivery of interfering RNA to the lung of a mammalian subject.

In some embodiments, this disclosure provides a method of treating a disease or disorder in a mammalian subject. A therapeutically effective amount of a composition of this disclosure containing an interfering RNA or a precursor thereof may be administered to a subject having a disease or disorder associated with expression or overexpression of a gene that can be reduced, decreased, downregulated, or silenced by the composition.

This disclosure encompasses methods for treating a disease of the lung such as respiratory distress, asthma, cystic fibrosis, pulmonary fibrosis, chronic obstructive pulmonary disease, bronchitis, or emphysema, by administering to the subject a therapeutically effective amount of a composition.

This disclosure encompasses methods for treating rheumatoid arthritis, liver disease, encephalitis, bone fracture, heart disease, viral disease including hepatitis and influenza, or cancer.

The compositions and methods of this disclosure may be administered to subjects by a variety of mucosal administration modes, including by oral, rectal, vaginal, intranasal, intrapulmonary, or transdermal delivery, or by topical delivery to the eyes, ears, skin or other mucosal surfaces. In some aspects of this disclosure, the mucosal tissue layer includes an epithelial cell layer. The epithelial cell can be pulmonary, tracheal, bronchial, alveolar, nasal, buccal, epidermal, or gastrointestinal. Compositions of this disclosure can be administered using conventional actuators such as mechanical spray devices, as well as pressurized, electrically activated, or other types of actuators.

Compositions of this disclosure may be administered in an aqueous solution as a nasal or pulmonary spray and may be dispensed in spray form by a variety of methods known to those skilled in the art. Pulmonary delivery of a composition of this disclosure is achieved by administering the composition in the form of drops, particles, or spray, which can be, e.g., aerosolized, atomized, or nebulized. Particles of the composition, spray, or aerosol can be in a either liquid or solid form. Preferred systems for dispensing liquids as a nasal spray are disclosed in U.S. Pat. No. 4,511,069. Such formulations may be conveniently prepared by dissolving compositions according to the present disclosure in water to produce an aqueous solution, and rendering said solution sterile. The formulations may be presented in multi-dose containers, e.g., in the sealed dispensing system disclosed in U.S. Pat. No. 4,511,069. Other suitable nasal spray delivery systems have been described in Transdermal Systemic Medication, Y. W. Chien ed., Elsevier Publishers, New York, 1985; and in U.S. Pat. No. 4,778,810. Additional aerosol delivery forms may include, e.g., compressed air-, jet-, ultrasonic-, and piezoelectric nebulizers, which deliver the biologically active agent dissolved or suspended in a pharmaceutical solvent, e.g., water, ethanol, or mixtures thereof.

Nasal and pulmonary spray solutions of the present disclosure typically comprise the drug or drug to be delivered, optionally formulated with a surface active agent, such as a nonionic surfactant (e.g., polysorbate-80), and one or more buffers. In some embodiments of the present disclosure, the nasal spray solution further comprises a propellant. The pH of the nasal spray solution may be from about pH 6.8 to 7.2. The pharmaceutical solvents employed can also be a slightly acidic aqueous buffer of pH 4-6. Other components may be added to enhance or maintain chemical stability, including preservatives, surfactants, dispersants, or gases.

In some embodiments, this disclosure is a pharmaceutical product which includes a solution containing a composition of this disclosure and an actuator for a pulmonary, mucosal, or intranasal spray or aerosol.

A dosage form of the composition of this disclosure can be liquid, in the form of droplets or an emulsion, or in the form of an aerosol.

A dosage form of the composition of this disclosure can be solid, which can be reconstituted in a liquid prior to administration. The solid can be administered as a powder. The solid can be in the form of a capsule, tablet or gel.

To formulate compositions for pulmonary delivery within the present disclosure, the biologically active agent can be combined with various pharmaceutically acceptable additives or delivery-enhancing components, as well as a base or carrier for dispersion of the active agent(s). Examples of additives or delivery-enhancing components include pH control agents such as arginine, sodium hydroxide, glycine, hydrochloric acid, citric acid, and mixtures thereof. Other additives or delivery-enhancing components include local anesthetics (e.g., benzyl alcohol), isotonizing agents (e.g., sodium chloride, mannitol, sorbitol), adsorption inhibitors (e.g., Tween 80), solubility enhancing agents (e.g., cyclodextrins and derivatives thereof), stabilizers (e.g., serum albumin), and reducing agents (e.g., glutathione). When the composition for mucosal delivery is a liquid, the tonicity of the formulation, as measured with reference to the tonicity of 0.9% (w/v) physiological saline solution taken as unity, is typically adjusted to a value at which no substantial, irreversible tissue damage will be induced in the mucosa at the site of administration. Generally, the tonicity of the solution is adjusted to a value of about ⅓ to 3, more typically ½ to 2, and most often ¾ to 1.7.

The biologically active agent may be dispersed in a base or vehicle, which may comprise a hydrophilic compound having a capacity to disperse the active agent and any desired additives. The base may be selected from a wide range of suitable carriers, including but not limited to, copolymers of polycarboxylic acids or salts thereof, carboxylic anhydrides (e.g. maleic anhydride) with other monomers (e.g., methyl(meth)acrylate, acrylic acid, etc.), hydrophilic vinyl polymers such as polyvinyl acetate, polyvinyl alcohol, polyvinylpyrrolidone, cellulose derivatives such as hydroxymethylcellulose, hydroxypropylcellulose, etc., and natural polymers such as chitosan, collagen, sodium alginate, gelatin, hyaluronic acid, and nontoxic metal salts thereof. A biodegradable polymer may be selected as a base or carrier, e.g., polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters, etc., can be employed as carriers. Hydrophilic polymers and other carriers can be used alone or in combination, and enhanced structural integrity can be imparted to the carrier by partial crystallization, ionic bonding, crosslinking and the like. The carrier can be provided in a variety of forms, including, fluid or viscous solutions, gels, pastes, powders, microspheres and films for direct application to the nasal mucosa. The use of a selected carrier in this context may result in promotion of absorption of the biologically active agent.

The biologically active agent can be combined with the base or carrier according to a variety of methods, and release of the active agent may be by diffusion, disintegration of the carrier, or associated formulation of water channels. In some circumstances, the active agent is dispersed in microcapsules (microspheres) or nanocapsules (nanospheres) prepared from a suitable polymer, e.g., isobutyl 2-cyanoacrylate (see, e.g., Michael, et al., J. Pharmacy Pharmacol. 43:1-5, 1991), and dispersed in a biocompatible dispersing medium applied to the nasal mucosa, which yields sustained delivery and biological activity over a protracted time.

Formulations for mucosal, nasal, or pulmonary delivery may contain a hydrophilic low molecular weight compound as a base or excipient. Such hydrophilic low molecular weight compounds provide a passage medium through which a water-soluble active agent, such as a physiologically active peptide or protein, may diffuse through the base to the body surface where the active agent is absorbed. The hydrophilic low molecular weight compound optionally absorbs moisture from the mucosa or the administration atmosphere and dissolves the water-soluble active peptide. The molecular weight of the hydrophilic low molecular weight compound is generally not more than 10,000 and preferably not more than 3000. Examples of hydrophilic low molecular weight compounds include polyol compounds, such as oligo-, di- and monosaccarides including sucrose, mannitol, lactose, L-arabinose, D-erythrose, D-ribose, D-xylose, D-mannose, D-galactose, lactulose, cellobiose, gentibiose, glycerin, polyethylene glycol, and mixtures thereof. Further examples of hydrophilic low molecular weight compounds include N-methylpyrrolidone, alcohols (e.g., oligovinyl alcohol, ethanol, ethylene glycol, propylene glycol, etc.), and mixtures thereof.

The compositions of this disclosure may alternatively contain as pharmaceutically acceptable carriers substances as required to approximate physiological conditions, such as pH adjusting and buffering agents, tonicity adjusting agents, and wetting agents, e.g., sodium acetate, sodium lactate, sodium chloride, potassium chloride, calcium chloride, sorbitan monolaurate, triethanolamine oleate, and mixtures thereof. For solid compositions, conventional nontoxic pharmaceutically acceptable carriers can be used which include, e.g., pharmaceutical grades of mannitol, lactose, starch, magnesium stearate, sodium saccharin, talcum, cellulose, glucose, sucrose, magnesium carbonate, and the like.

In certain embodiments of this disclosure, the biologically active agent may be administered in a time release formulation, e.g., in a composition which includes a slow release polymer. The active agent can be prepared with carriers that will protect against rapid release, e.g., a controlled release vehicle such as a polymer, microencapsulated delivery system or bioadhesive gel. Prolonged delivery of the active agent, in various compositions of this disclosure can be brought about by including in the composition agents that delay absorption, e.g., aluminum monosterate hydrogels and gelatin.

Within certain embodiments of this disclosure, the siNA composition may contain one or more natural or synthetic surfactants. Certain natural surfactants are found in human lung (pulmonary surfactant), and are a complex mixture of phospholipids and proteins that form a monolayer at the alveolar air-liquid interface and reduces surface tension to near zero at expiration and prevents alveolar collapse. Over 90% (by weight) of pulmonary surfactant is composed of phospholipids with approximately 40-80% being DPPC and the remainder being unsaturated phosphatidylcholines POPG, POPC and phosphatidylglycerols. The remaining 10% (by weight) of surfactant is composed of plasma proteins and apoproteins, such as surface proteins (SP)-A, SP-B, SP-C and SP-D.

Examples of natural surfactants that may be used in this disclosure include SURVANTA™ (beractant), CUROSURF™ (poractant alfa) and INFASURF™ (calfactant), and mixtures thereof.

Examples of synthetic surfactants include sinapultide; a combination of dipalmitoylphosphatidylcholine, palmitoyloleoyl phosphatidylglycerol and palmitic acid; SURFAXIN™ (lucinactant); and EXOSURF™ (colfosceril); components which may contain tyloxapol, DPPC, and hexadecanol; and mixtures thereof.

The nucleic acid component, lipids, and any additional components may be mixed together first in a suitable medium such as a cell culture medium, after which one or more additional lipids or compounds may be added to the mixture. Alternatively, the lipids can be mixed together first in a suitable medium such as a cell culture medium, after which the nucleic acid component can be added.

Nucleic acid molecules can be administered within formulations that include one or more additional components, such as a pharmaceutically acceptable carrier, diluent, excipient, adjuvant, emulsifier, buffer, stabilizer, or preservative.

As used herein, the term “carrier” means a pharmaceutically acceptable solid or liquid filler, solvent, diluent or encapsulating material. A water-containing liquid carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. Examples of ingredients of the above categories can be found in the U.S. Pharmacopeia National Formulary, 1990, pp. 1857-1859, as well as in Raymond C. Rowe, et al., Handbook of Pharmaceutical Excipients, 5th Ed., 2006, and “Remington: The Science and Practice of Pharmacy,” 21st Ed., 2006, editor David B. Troy.

Examples of preservatives include phenol, methyl paraben, paraben, m-cresol, thiomersal, benzylalkonium chloride, and mixtures thereof.

Examples of surfactants include oleic acid, sorbitan trioleate, polysorbates, lecithin, phosphotidylcholines, various long chain diglycerides and phospholipids, and mixtures thereof.

Examples of phospholipids include phosphatidylcholine, lecithin, phosphatidylglycerol, phosphatidylinositol, phosphatidylserine, and phosphatidylethanolamine, and mixtures thereof.

Examples of dispersants include ethylenediaminetetraacetic acid.

Examples of gases include nitrogen, helium, chlorofluorocarbons (CFCs), hydrofluorocarbons (HFCs), carbon dioxide, air, and mixtures thereof.

As used herein, and among other meanings, the term “ligand” refers to any compound or molecule, such as a drug, peptide, hormone, or neurotransmitter, that is capable of interacting with another compound, such as a receptor, either directly or indirectly. The receptor that interacts with a ligand can be present on the surface of a cell or can alternately be an intracellular receptor. Interaction of the ligand with the receptor can result in a biochemical reaction, or can simply be a physical interaction or association.

By “asymmetric hairpin” as used herein is meant a linear siNA molecule comprising an antisense region, a loop portion that can comprise nucleotides or non-nucleotides, and a sense region that comprises fewer nucleotides than the antisense region to the extent that the sense region has enough complementary nucleotides to base pair with the antisense region and form a duplex with loop. For example, an asymmetric hairpin siNA molecule of this disclosure can comprise an antisense region having length sufficient to mediate RNAi in a T-cell (e.g., about 19 to about 22 (e.g., about 19, 20, 21, or 22) nucleotides) and a loop region comprising about 4 to about 8 (e.g., about 4, 5, 6, 7, or 8) nucleotides, and a sense region having about 3 to about 18 (e.g., about 3, 4, 5, 6, 7, 8, 9, 10, 11, 12, 13, 14, 15, 16, 17, or 18) nucleotides that are complementary to the antisense region. The asymmetric hairpin siNA molecule can also comprise a 5′-terminal phosphate group that can be chemically modified. The loop portion of the asymmetric hairpin siNA molecule can comprise nucleotides, non-nucleotides, linker molecules, or conjugate molecules as described herein.

By “asymmetric duplex” as used herein is meant an siNA molecule having two separate strands comprising a sense region and an antisense region, wherein the sense region comprises fewer nucleotides than the antisense region to the extent that the sense region has enough complementary nucleotides to base pair with the antisense region and form a duplex. For example, an asymmetric duplex siNA molecule of this disclosure can comprise an antisense region having length sufficient to mediate RNAi in a T-cell (e.g., about 19 to about 22 (e.g., about 19, 20, 21, or 22) nucleotides) and a sense region having about 3 to about 18 (e.g., about 3, 4, 5, 6, 7, 8, 9, 10, 11, 12, 13, 14, 15, 16, 17, or 18) nucleotides that are complementary to the antisense region.

By “modulate gene expression” is meant that the expression of a target gene is upregulated or downregulated, which can include upregulation or downregulation of mRNA levels present in a cell, or of mRNA translation, or of synthesis of protein or protein subunits, encoded by the target gene. Modulation of gene expression can be determined also be the presence, quantity, or activity of one or more proteins or protein subunits encoded by the target gene that is up regulated or down regulated, such that expression, level, or activity of the subject protein or subunit is greater than or less than that which is observed in the absence of the modulator (e.g., a dsRNA). For example, the term dose “modulate” can mean “inhibit,” but the use of the word “modulate” is not limited to this definition.

By “inhibit,” “down-regulate,” or “reduce” expression, it is meant that the expression of the gene, or level of RNA molecules or equivalent RNA molecules encoding one or more proteins or protein subunits, or level or activity of one or more proteins or protein subunits encoded by a target gene, is reduced below that observed in the absence of the nucleic acid molecules (e.g., siNA) of this disclosure. In one embodiment, inhibition, down-regulation or reduction with an siNA molecule is below that level observed in the presence of an inactive or attenuated molecule. In another embodiment, inhibition, down-regulation, or reduction with siNA molecules is below that level observed in the presence of, e.g., an siNA molecule with scrambled sequence or with mismatches. In another embodiment, inhibition, down-regulation, or reduction of gene expression with a nucleic acid molecule of the instant disclosure is greater in the presence of the nucleic acid molecule than in its absence.

Gene “silencing” refers to partial or complete loss-of-function through targeted inhibition of gene expression in a cell and may also be referred to as “knock down.” Depending on the circumstances and the biological problem to be addressed, it may be preferable to partially reduce gene expression. Alternatively, it might be desirable to reduce gene expression as much as possible. The extent of silencing may be determined by methods known in the art, some of which are summarized in International Publication No. WO 99/32619. Depending on the assay, quantification of gene expression permits detection of various amounts of inhibition that may be desired in certain embodiments of this disclosure, including prophylactic and therapeutic methods, which will be capable of knocking down target gene expression, in terms of mRNA levels or protein levels or activity, e.g., by equal to or greater than 10%, 30%, 50%, 75% 90%, 95% or 99% of baseline (i.e., normal) or other control levels, including elevated expression levels as may be associated with particular disease states or other conditions targeted for therapy.

The phrase “inhibiting expression of a target gene” refers to the ability of an siNA of this disclosure to initiate gene silencing of the target gene. To examine the extent of gene silencing, samples or assays of the organism of interest or cells in culture expressing a particular construct are compared to control samples lacking expression of the construct. Control samples (lacking construct expression) are assigned a relative value of 100%. Inhibition of expression of a target gene is achieved when the test value relative to the control is about 90%, often 50%, and in certain embodiments 25-0%. Suitable assays include, e.g., examination of protein or mRNA levels using techniques known to those of skill in the art such as dot blots, northern blots, in situ hybridization, ELISA, immunoprecipitation, enzyme function, as well as phenotypic assays known to those of skill in the art.

By “subject” is meant an organism, tissue or cell, which may include an organism as the subject or as a donor or recipient of explanted cells or the cells that are themselves subjects for siNA delivery. “Subject” therefore may refers to an organism, organ, tissue or cell, including in vitro or ex vivo organ, tissue or cellular subjects, to which the nucleic acid molecules of this disclosure can be administered and enhanced by delivery peptides described herein. Exemplary subjects include mammalian individuals or cells, e.g., human patients or cells.

As used herein “cell” is used in its usual biological sense, and does not refer to an entire multicellular organism, e.g., specifically does not refer to a human. The cell can be present in an organism, e.g., birds, plants and mammals such as humans, cows, sheep, apes, monkeys, swine, dogs and cats. The cell can be prokaryotic (e.g., bacterial cell) or eukaryotic (e.g., mammalian or plant cell). The cell can be of somatic or germ line origin, totipotent or pluripotent, dividing or non-dividing. The cell can also be derived from or can comprise a gamete or embryo, a stem cell, or a fully differentiated cell.

By “vectors” is meant any nucleic acid- and/or viral-based technique used to deliver a desired nucleic acid.

By “RNA” is meant a molecule comprising at least one ribonucleotide residue. By “ribonucleotide” is meant a nucleotide with a hydroxyl group at the 2′ position of a .beta.-D-ribo-furanose moiety. The term “RNA” includes double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siNA or internally, e.g., at one or more nucleotides of the RNA. Nucleotides in the RNA molecules of this disclosure can also comprise non-standard nucleotides, such as non-naturally occurring nucleotides or chemically synthesized nucleotides or deoxynucleotides. These altered RNAs can be referred to as analogs or analogs of naturally-occurring RNA.

By “highly conserved sequence region” is meant, a nucleotide sequence of one or more regions in a target gene does not vary significantly from one generation to the other or from one biological system to the other.

By “sense region” is meant a nucleotide sequence of an siNA molecule having complementarity to an antisense region of the siNA molecule. In addition, the sense region of an siNA molecule can comprise a nucleic acid sequence having homology with a target nucleic acid sequence.

By “antisense region” is meant a nucleotide sequence of an siNA molecule having complementarity to a target nucleic acid sequence. In addition, the antisense region of an siNA molecule can optionally comprise a nucleic acid sequence having complementarity to a sense region of the siNA molecule.

By “target nucleic acid” is meant any nucleic acid sequence whose expression or activity is to be modulated. The target nucleic acid can be DNA or RNA.

By “complementarity” is meant that a nucleic acid can form hydrogen bond(s) with another nucleic acid sequence by either traditional Watson-Crick or other non-traditional types. In reference to the nucleic molecules of the present disclosure, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner, et al., CSH Symp. Quant. Biol. LII, pp. 123-133, 1987; Frier, et al., Proc. Nat. Acad. Sci. USA 83:9373-9377, 1986; Turner, et al., J. Am. Chem. Soc. 109:3783-3785, 1987). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, or 10 nucleotides out of a total of 10 nucleotides in the first oligonucleotide being based paired to a second nucleic acid sequence having 10 nucleotides represents 50%, 60%, 70%, 80%, 90%, and 100% complementary respectively). “Perfectly complementary” means that all the contiguous residues of a nucleic acid sequence will hydrogen bond with the same number of contiguous residues in a second nucleic acid sequence.

The term “universal base” as used herein refers to nucleotide base analogs that form base pairs with each of the natural DNA/RNA bases with little discrimination between them. Non-limiting examples of universal bases include C-phenyl, C-naphthyl and other aromatic derivatives, inosine, azole carboxamides and nitroazole derivatives such as 3-nitropyrrole, 4-nitroindole, 5-nitroindole and 6-nitroindole as known in the art (see, e.g., Loakes, Nucleic Acids Research 29:2437-2447, 2001).

The term “acyclic nucleotide” as used herein refers to any nucleotide having an acyclic ribose sugar, e.g., where any of the ribose carbons (C1, C2, C3, C4, or C5), are independently or in combination absent from the nucleotide.

The term “biodegradable” as used herein, refers to degradation in a biological system, e.g., enzymatic degradation or chemical degradation.

The term “biologically active molecule” as used herein, refers to compounds or molecules that are capable of eliciting or modifying a biological response in a system. Non-limiting examples of biologically active siNA molecules either alone or in combination with other molecules contemplated by the instant disclosure include therapeutically active molecules such as antibodies, cholesterol, hormones, antivirals, peptides, proteins, chemotherapeutics, small molecules, vitamins, co-factors, nucleosides, nucleotides, oligonucleotides, enzymatic nucleic acids, antisense nucleic acids, triplex forming oligonucleotides, 2,5-A chimeras, siNA, dsRNA, allozymes, aptamers, decoys and analogs thereof. Biologically active molecules of this disclosure also include molecules capable of modulating the pharmacokinetics and/or pharmacodynamics of other biologically active molecules, e.g., lipids and polymers such as polyamines, polyamides, polyethylene glycol and other polyethers.

The term “phospholipid” as used herein, refers to a hydrophobic molecule comprising at least one phosphorus group. For example, a phospholipid can comprise a phosphorus-containing group and saturated or unsaturated alkyl group, optionally substituted with OH, COOH, oxo, amine, or substituted or unsubstituted aryl groups.

By “cap structure” is meant chemical modifications, which have been incorporated at either terminus of the oligonucleotide (see, e.g., Adamic, et al., U.S. Pat. No. 5,998,203, incorporated by reference herein). These terminal modifications protect the nucleic acid molecule from exonuclease degradation, and may help in delivery and/or localization within a cell. The cap may be present at the 5′-terminus (5′-cap) or at the 3′-terminal (3′-cap) or may be present on both termini. In non-limiting examples, the 5′-cap includes, but is not limited to, glyceryl, inverted deoxy abasic residue (moiety); 4′,5′-methylene nucleotide; 1-(beta-D-erythrofuranosyl) nucleotide, 4′-thio nucleotide; carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety.

Non-limiting examples of the 3′-cap include, but are not limited to, glyceryl, inverted deoxy abasic residue (moiety), 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details, see Beaucage and Lyer, Tetrahedron 49:1925, 1993; incorporated by reference herein).

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine and therefore lacks a base at the 1′-position.

By “nucleotide” as used herein is as recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see, e.g., Usman and McSwiggen, supra; Eckstein, et al., International PCT Publication No. WO 92/07065; Usman et al, International PCT Publication No. WO 93/15187; Uhlman & Peyman, supra, all are hereby incorporated by reference herein). There are several examples of modified nucleic acid bases known in the art as summarized by Limbach, et al, Nucleic Acids Res. 22:2183, 1994. Some of the non-limiting examples of base modifications that can be introduced into nucleic acid molecules include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and others (Burgin, et al., Biochemistry 35:14090, 1996; Uhlman & Peyman, supra). By “modified bases” in this aspect is meant nucleotide bases other than adenine, guanine, cytosine and uracil at 1′ position or their equivalents.

By “target site” is meant a sequence within a target RNA that is “targeted” for cleavage mediated by an siNA construct which contains sequences within its antisense region that are complementary to the target sequence.

By “detectable level of cleavage” is meant cleavage of target RNA (and formation of cleaved product RNAs) to an extent sufficient to discern cleavage products above the background of RNAs produced by random degradation of the target RNA. Production of cleavage products from 1-5% of the target RNA is sufficient to detect above the background for most methods of detection.

By “biological system” is meant, material, in a purified or unpurified form, from biological sources, including but not limited to human, animal, plant, insect, bacterial, viral or other sources, wherein the system comprises the components required for RNAi activity. The term “biological system” includes, e.g., a cell, tissue, or organism, or extract thereof. The term biological system also includes reconstituted RNAi systems that can be used in an in vitro setting.

The term “biodegradable linker” as used herein, refers to a nucleic acid or non-nucleic acid linker molecule that is designed as a biodegradable linker to connect one molecule to another molecule, e.g., a biologically active molecule to an siNA molecule of this disclosure or the sense and antisense strands of an siNA molecule of this disclosure. The biodegradable linker is designed such that its stability can be modulated for a particular purpose, such as delivery to a particular tissue or cell type. The stability of a nucleic acid-based biodegradable linker molecule can be modulated by using various chemistries, e.g., combinations of ribonucleotides, deoxyribonucleotides, and chemically-modified nucleotides, such as 2′-O-methyl, 2′-fluoro, 2′-amino, 2′-O-amino, 2′-C-allyl, 2′-O-allyl, and other 2′-modified or base modified nucleotides. The biodegradable nucleic acid linker molecule can be a dimer, trimer, tetramer or longer nucleic acid molecule, e.g., an oligonucleotide of about 2, 3, 4, 5, 6, 7, 8, 9, 10, 11, 12, 13, 14, 15, 16, 17, 18, 19, or 20 nucleotides in length, or can comprise a single nucleotide with a phosphorus-based linkage, e.g., a phosphoramidate or phosphodiester linkage. The biodegradable nucleic acid linker molecule can also comprise nucleic acid backbone, nucleic acid sugar, or nucleic acid base modifications.

By “abasic” is meant sugar moieties lacking a base or having other chemical groups in place of a base at the 1′ position, see, e.g., Adamic, et al., U.S. Pat. No. 5,998,203.

By “unmodified nucleoside” is meant one of the bases adenine, cytosine, guanine, thymine, or uracil joined to the 1′ carbon of beta.-D-ribo-furanose.

By “modified nucleoside” is meant any nucleotide base which contains a modification in the chemical structure of an unmodified nucleotide base, sugar and/or phosphate. Non-limiting examples of modified nucleotides are shown by Formulae I-VII and/or other modifications described herein.

In connection with 2′-modified nucleotides as described for the present disclosure, by “amino” is meant 2′-NH₂ or 2′-O-NH₂, which can be modified or unmodified. Such modified groups are described, e.g., in Eckstein, et al., U.S. Pat. No. 5,672,695 and Matulic-Adamic, et al., U.S. Pat. No. 6,248,878.

The siNA molecules can be complexed with cationic lipids, packaged within liposomes, or otherwise delivered to target cells or tissues. The nucleic acid or nucleic acid complexes can be locally administered to through injection, infusion pump or stent, with or without their incorporation in biopolymers. In another embodiment, polyethylene glycol (PEG) can be covalently attached to siNA compounds of the present disclosure, to the delivery peptide or both. The attached PEG can be any molecular weight, preferably from about 500 to about 50,000 daltons (Da).

The sense region can be connected to the antisense region via a linker molecule, such as a polynucleotide linker or a non-nucleotide linker.

“Inverted repeat” refers to a nucleic acid sequence comprising a sense and an antisense element positioned so that they are able to form a double stranded siRNA when the repeat is transcribed. The inverted repeat may optionally include a linker or a heterologous sequence such as a self-cleaving ribozyme between the two elements of the repeat. The elements of the inverted repeat have a length sufficient to form a double stranded RNA. Typically, each element of the inverted repeat is about 15 to about 100 nucleotides in length, preferably about 20-30 base nucleotides, preferably about 20-25 nucleotides in length, e.g., 20, 21, 22, 23, 24, 25, 26, 27, 28, 29, or 30 nucleotides in length.

“Nucleic acid” refers to deoxyribonucleotides or ribonucleotides and polymers thereof in single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

“Large double-stranded RNA” refers to any double-stranded RNA having a size greater than about 40 base pairs (bp) e.g., larger than 100 bp or more particularly larger than 300 bp. The sequence of a large dsRNA may represent a segment of an mRNA or the entire mRNA. The maximum size of the large dsRNA is not limited herein. The double-stranded RNA may include modified bases where the modification may be to the phosphate sugar backbone or to the nucleoside. Such modifications may include a nitrogen or sulfur heteroatom or any other modification known in the art.

The double-stranded structure may be formed by self-complementary RNA strand such as occurs for a hairpin or a micro RNA or by annealing of two distinct complementary RNA strands.

“Overlapping” refers to when two RNA fragments have sequences which overlap by a plurality of nucleotides on one strand, e.g., where the plurality of nucleotides (nt) numbers as few as 2-5 nucleotides or by 5-10 nucleotides or more.

“One or more dsRNAs” refers to dsRNAs that differ from each other on the basis of sequence.

“Target gene or mRNA” refers to any gene or mRNA of interest. Indeed any of the genes previously identified by genetics or by sequencing may represent a target. Target genes or mRNA may include developmental genes and regulatory genes as well as metabolic or structural genes or genes encoding enzymes. The target gene may be expressed in those cells in which a phenotype is being investigated or in an organism in a manner that directly or indirectly impacts a phenotypic characteristic. The target gene may be endogenous or exogenous. Such cells include any cell in the body of an adult or embryonic animal or plant including gamete or any isolated cell such as occurs in an immortal cell line or primary cell culture.

All publications, references, patents, patent publications and patent applications cited herein are each hereby specifically incorporated by reference in entirety.

While this disclosure has been described in relation to certain embodiments, and many details have been set forth for purposes of illustration, it will be apparent to those skilled in the art that this disclosure includes additional embodiments, and that some of the details described herein may be varied considerably without departing from this disclosure. This disclosure includes such additional embodiments, modifications and equivalents. In particular, this disclosure includes any combination of the features, terms, or elements of the various illustrative components and examples.

The use herein of the terms “a,” “an,” “the” and similar terms used in this disclosure, and in the claims, are to be construed to include both the singular and the plural. The terms “comprising,” “having,” “including,” and “containing” are to be construed as open-ended terms which mean, e.g., “including, but not limited to.” Recitation of a range of values herein refers individually to each and any separate value falling within the range as if it were individually recited herein, whether or not some of the values within the range are expressly recited. For example, the range “4 to 12” includes without limitation the values 5, 5.1, 5.35 and any other whole, integral, rational, irrational or fractional value greater than or equal to 4 and less than or equal to 12, including 6, 7, 8, 9, 10, and 11. Specific values employed herein will be understood as exemplary and not to limit the scope of this disclosure.

Definitions of technical terms provided herein should be construed to include without recitation those meanings associated with these terms known to those skilled in the art, and are not intended to limit the scope of this disclosure. Definitions of technical terms provided herein shall be construed to dominate over alternative definitions in the art or definitions which become incorporated herein by reference to the extent that the alternative definitions conflict with the definition provided herein.

The examples given herein, and the exemplary language used herein are solely for the purpose of illustration, and are not intended to limit the scope of this disclosure.

When a list of examples is given, such as a list of compounds or molecules suitable for this disclosure, it will be apparent to those skilled in the art that mixtures or combinations of the listed compounds or molecules are also suitable.

### EXAMPLE 1

### Synthesis of dsRNA Peptide Conjugates

This example illustrates the general methodology used to synthesize of dsRNA-peptide conjugates. Both peptides and RNAs are prepared using standard solid phase synthesis methods. The peptide and RNA molecules must be functionalized with specific moieties to allow for covalent attachment to each other. For the peptide, the N-terminus is functionalized with 3-maleimidopropionic acid. However, it is recognized that other functional groups such as bromo or iodoacetyl moieties will work as well. For the RNA molecule 5′ end of the sense (“sen”) strand or 5′ end of the antisense (“asen”) strand is functionalized with a 1-O-dimethoxytrityl-hexyl-disulfide linker.

The RNA oligonucleotide was dissolved in 0.5 mL of water to which was added 2 mL of buffer A (20 mM Tris-HCl, pH 6.8, 50% formamide). The peptide was subsequently added which resulted in the formation of a precipitate that was solubilized by the additional 1 mL of 2 M TEAA buffer. Upon completion of the reaction, the solvent was removed under reduced pressure and the resulting solid was dissolved in buffer A (20 mM Tris-HCl, pH 6.8, 50% formamide). The material was loaded onto an Amersham Resource Q column and washed with five column volumes of buffer A at 6 mL/min. Separation was accomplished by running a linear gradient of buffer B (20 mM Tris-HCl, pH 6.8, 50% formamide, 1 M NaCl) from 15-60% for 15 column volumes at a flow rate of 6 mL/min. The purified conjugate was desalted by slidalyzer dialysis cassettes (10K MWCO) against PBS. The amount of the conjugate was determined spectrophotometrically based on the calculated molar absorption coefficient at 260 nm. Analysis of the purity of this conjugate by RP-HPLC is shown below.

The RNA strand conjugated to the peptide was annealed to its complimentary RNA strand in 50 mM potassium acetate, 1 mM magnesium acetate and 15 mM HEPES, pH 7.4 and heating at 90° C. for two minutes followed by incubation at 37° C. for one hour. The formation of the double stranded RNA conjugate was confirmed by non denaturing (15%) polyacrylamide gel electrophoresis and ethidium bromide staining.

Table 4 below summarizes peptides synthesized for preparing dsRNA peptide conjugates.

TABLE 4

Peptides for Preparation of dsRNA Peptide
Conjugates

| | |
|---|---|
| Peptide Name | Amino Acid Sequence |

| | |
|---|---|
| PN277 | KGSKKAVTKAQKKDGKKRKRSRKESYSVYVYKVLKQ |
| | (SEQ ID NO: 27) |
| PN857 | KGSKKAVTKAQKKEGKKRKRSRKESYSVYVYKVLKQ |
| | (SEQ ID NO: 28) |
| PN828 | AQKKEGKKRKRSRKESYSVYVYKVLKQ |
| | (SEQ ID NO: 29) |
| PN750 | KRSRKESYSVYVYKVLKQ |
| | (SEQ ID NO: 30) |
| PN872 | PEG (200 Da)-KRSRKESYSVYVYKVLKQ |
| | (SEQ ID NO: 31) |
| PN963 | ESYSVYVYKVLKQ |
| | (SEQ ID NO: 32) |
| PN751 | YKVLKQ |
| | (SEQ ID NO: 33) |
| PN3072 | RVIRWFQNKRSKDKK |
| | (SEQ ID NO: 34) |
| PN3073 | GALFLGFLGAAGSTMGAWSQPKSKRKV |
| | (SEQ ID NO: 35) |
| PN3070 | RQIKIWFQNRRMKWKK |
| | (SEQ ID NO: 36) |
| PN3185 | RQIKIWFQNRRMKWKK (all D-amino acids) |
| | (SEQ ID NO: 37) |
| PN3071 | GWTLNSAGYLLKINLKALAALAKKIL |
| | (SEQ ID NO: 38) |
| PN3414 | LLNQLAGRMIPKWSQKSKRKV |
| | (SEQ ID NO: 39) |
| PN3415 | TLDHVLDHVQTWSQKSKRKV |
| | (SEQ ID NO: 40) |
| PN3416 | SYFILRRRRKRFPYFFTDVRVAA |
| | (SEQ ID NO: 41) |
| PN3079 | RRRRRRRRRR |
| | (SEQ ID NO: 42) |
| PN3671 | RRRRRRRR (D-amino acids) |
| | (SEQ ID NO: 43) |
| PN2986 | KETWWETWWTEWSQPGRKKRRQRRRPPQ |
| | (SEQ ID NO: 44) |
| PN740 | GRPRESGKKRKRKRLKP |
| | (SEQ ID NO: 45) |
| PN3846 | KSYSVYVYKVLKQ |
| | (SEQ ID NO: 46) |
| PN3847 | ESYSVYVYRVLRQ |
| | (SEQ ID NO: 47) |
| PN3848 | RSYSVYVYRVLRQ |
| | (SEQ ID NO: 48) |
| PN3884 | QKLVKYVYVSYSE |
| | (SEQ ID NO: 49) |
| PN3885 | ESYSVYVYKVLKQ (all D amino acids) |
| | (SEQ ID NO: 50) |
| PN3886 | ASYSVYVYAVLAQ |
| | (SEQ ID NO: 51) |
| PN3889 | QKLVKYVYVSYSE (all D amino acids) |
| | (SEQ ID NO: 52) |
| PN3948 | ESYSVYVYKVLKQ-Peg(600 Da) |
| | (SEQ ID NO: 53) |
| PN3980 | RRRRRRESYSVYVYKVLKQ |
| | (SEQ ID NO: 54) |
| PN3981 | ESYSVYVYKVLKQRRRRRR |
| | (SEQ ID NO: 55) |
| PN3982 | RRRRRRRQIKIWFQNRRMKWKK |
| | (SEQ ID NO: 56) |
| PN3983 | RQIKIWFQNRRMKWKKRRRRRR |
| | (SEQ ID NO: 57) |
| PN3984 | KTKIESLKEHGRRRRRR |
| | (SEQ ID NO: 58) |
| PN3985 | MDVNPTLLFLKVPAQNAISTTFPYTRRRRRR |
| | (SEQ ID NO: 59) |
| PN3986 | GLFEALLELLESLWELLLEARRRRRR |
| | (SEQ ID NO: 60) |
| PN3987 | LLNQLAGRMIPKRRRRRR |
| | (SEQ ID NO: 61) |
| PN3988 | TLDHVLDHVQTRRRRRR |
| | (SEQ ID NO: 62) |
| PN3989 | GLFGAIAGFIENGWEGMIDGRRRRRR |
| | (SEQ ID NO: 63) |
| PN3990 | KETWWETWWTERRRRRR |
| | (SEQ ID NO: 64) |
| PN3991 | HHHHHHHHHHRRRRRR |
| | (SEQ ID NO: 65) |
| PN3992 | AAVALLPAVLLALLAPRRRRRR |
| | (SEQ ID NO: 66) |

Table 5 below summarizes the dicer substrate ribonucleic acid sequences used to prepare dicer substrate peptide conjugates. The sense strand of an RNA is identified as “sen” and the antisense strand of an RNA is identified as “asen”. CN952sen and CN950asen are complementary strands, CN740 and CN741 are complementary strands, G1498dicersen and G1498dicerasen are complementary strands, N168sen and N168asen are complementary strands, N169sen and N169asen are complementary strands and mTNFα-S1sen and mTNFα-S1asen are complementary strands. Complementary strands are annealed to generate dsRNAs.

TABLE 5

RNAs Used to Prepare Dicer Substrate Peptide
Conjugates

| | | SEQ ID |
|---|---|---|
| Name | Nucleic Acid Sequence | NO: |
| CN952sen | CCCAUGUGCUCCUCACCCACACCdAT | 67 |
| (N163sen) | | |
| CN950asen | AUGGUGUGGGUGAGGAGCACAUGGGUG | 68 |
| (N163asen) | | |
| CN740sen | GCCUGUACCUCAUCUACUCCCAGGUCC | 69 |
| (DS1sen) | | |
| CN741asen | GGUCCUGGGAGUAGAUGAGGUACAGGCUU | 70 |
| (DS1asen) | | |
| G1498dicersen | GGAUCUUAUUUCUUCGGAGACAAdTdG | 71 |
| (2848sen) | | |
| G1498dicerasen | CAUUGUCUCCGAAGAAAUAAGAUCCUU | 72 |
| (3063asen) | | |
| N168sen | GGGUCGGAACCCAAGCUUAGAACdTdT | 73 |
| N168asen | AAGUUCUAAGCUUGGGUUCCGACCCUA | 74 |
| N169sen | GACUCAGCGCUGAGAUCAAUCGGdCdC | 75 |
| N169asen | GGCCGAUUGAUCUCAGCGCUGAGUCGG | 76 |
| mTNFα-S1sen | GUCUCAGCCUCUUCUCAUUCCUGdCdT | 77 |
| mTNFα-S1asen | AGCAGGAAUGAGAAGAGGCUGAGACAU | 78 |
| LacZsen | CUACACAAAUCAGCGAUUUCCAUdGT | 79 |
| LacZasen | ACAUGGAAAUCGCUGAUUUGUGUAGUC | 80 |
| PPIBsen | GGAAAGACUGUUCCAAAAACAGUdGdG | 81 |
| PPIBasen | CCACUGUUUUUGGAACAGUCUUUCCUU | 82 |

### EXAMPLE 2

### Preparation of Conjugate 1

Preparation of Conjugate 1 (dsCoP277nfR950): Conjugation of peptide PN277 (H2B 13-48) (SEQ ID NO:27) and oligonucleotide CN950asen (N163asen) (SEQ ID NO:68) having the following structure (complementary sense strand N163sen (CCCAUGUGCUCCUCACCCACACCdAT) (SEQ ID NO:83) of the RNA is not shown):

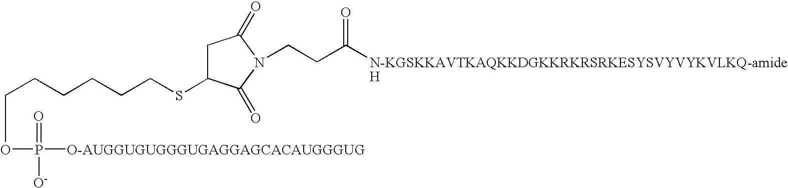

Oligonucleotide CN950asen was dissolved in 0.5 mL of water to which was added 2 mL of buffer A (20 mM Tris-HCl, pH 6.8, 50% formamide). Peptide PN277 was subsequently added which resulted in the formation of a precipitate that was solubilized by the additional 1 mL of 2 M TEAA buffer. Upon completion of the reaction, the solvent was removed under reduced pressure and the resulting solid was dissolved in buffer A (20 mM Tris-HCl, pH 6.8, 50% formamide). The material was loaded onto an Amersham Resource Q column and washed with 5 column volumes of buffer A at 6 mL/min. Separation was accomplished by running a linear gradient of buffer B (20 mM Tris-HCl, pH 6.8, 50% formamide, 1 M NaCl) from 15-60% for 15 column volumes at a flow rate of 6 mL/min. The purified conjugate was desalted by slidalyzer dialysis cassettes (10K MWCO) against PBS. The amount of the conjugate was determined spectrophotometrically based on the calculated molar absorption coefficient at λ=260 nm. Purity of the conjugate was confirmed by RP-HPLC.

The antisense strand of the RNA peptide conjugate was annealed to its complimentary sense strand in 50 mM potassium acetate, 1 mM magnesium acetate and 15 mM HEPES pH 7.4 by heating at 90° C. for 2 min followed by incubation at 37° C. for 1 h. The formation of the double stranded RNA conjugate was confirmed by non denaturing (15%) polyacrylamide gel electrophoresis and staining with ethidium bromide.

### EXAMPLE 3

### Preparation of Conjugate 2

Preparation of Conjugate 2 (dsCoP277nfR952): Conjugation of peptide PN277 (H2B 13-48) (SEQ ID NO:27) and oligonucleotide CN952sen (N163sen) (SEQ ID NO:67) having the following structure (complementary antisense strand N163asen (AUGGUGUGGGUGAGGAGCACAUGGGUG) (SEQ ID NO:84) of the RNA is not shown):

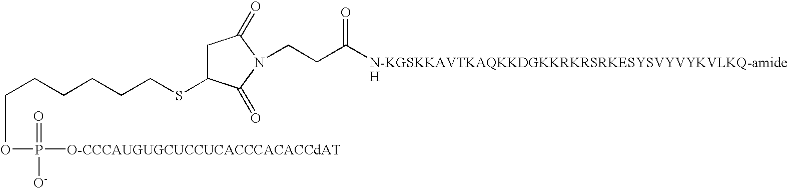

The RNA strand conjugated to the peptide was annealed to its complimentary RNA strand (N163asen). Conjugate 2 was made using the methods and procedures described above in Example 1.

### EXAMPLE 4

### Preparation of Conjugate 3

Preparation of Conjugate 3 (dsCoP277nfR740): Conjugation of peptide PN277 (H2B 13-48) (SEQ IS NO:27) and oligonucleotide CN740sen (SEQ ID NO:69) having the following structure (complementary antisense strand CN741asen (GGUCCUGGGAGUAGAUGAGGUACAGGCUU) (SEQ ID NO:85) of the RNA is not shown):

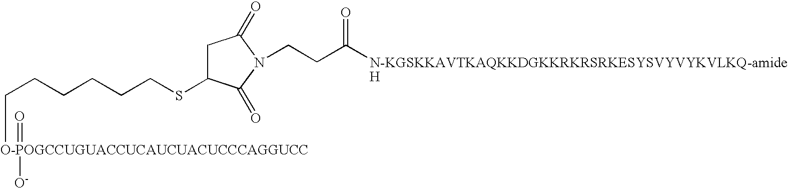

The RNA strand conjugated to the peptide was annealed to its complimentary RNA strand (CN741asen). Conjugate 3 was made using the methods and procedures described above in Example 1.

### EXAMPLE 5

### Preparation of Conjugate 4

Preparation of Conjugate 4 (dsCoP277nfR741): Conjugation of peptide PN277 (H2B 13-48) (SEQ ID NO:27) and oligonucleotide CN741asen (SEQ ID NO:70) having the following structure (complementary sense strand CN740sen (GCCUGUACCUCAUCUACUCCCAGGUCC) (SEQ ID NO:86) of the RNA not shown):

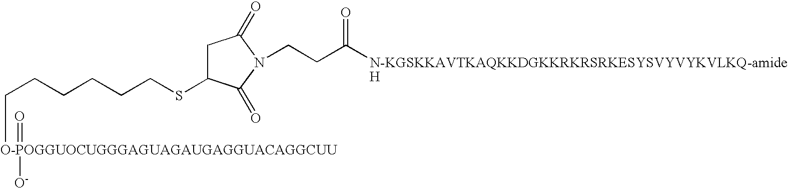

The RNA strand conjugated to the peptide was annealed to its complimentary RNA strand (CN740sen). Conjugate 4 was made using the methods and procedures described above in Example 1.

### EXAMPLE 6

### Preparation of Conjugate 5

Preparation of Conjugate 5 (dsCoP857nfR1280): Conjugation of peptide PN857 (H2B 13-48; Asp14 to Glu substitution) (SEQ ID NO:28) and oligonucleotide CN950asen (N163asen) (SEQ ID NO:68) having the following structure (complementary sense strand N163sen (CCCAUGUGCUCCUCACCCACACCdAT) (SEQ ID NO:87) of the RNA is not shown):

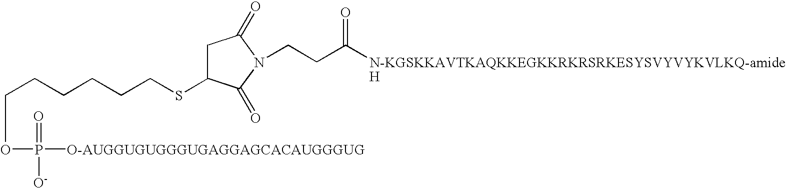

The RNA strand conjugated to the peptide was annealed to its complimentary RNA strand (N163sen). Conjugate 5 was made using the methods and procedures described above in Example 1.

### EXAMPLE 7

### Preparation of Conjugate 6

Preparation of Conjugate 6 (dsCoP857nfR1277): Conjugation of peptide PN857 (H2B 13-48; Asp14 to Glu substitution) (SEQ ID NO:28) and oligonucleotide N168asen (SEQ ID NO:74) having the following structure (complementary sense strand N168sen (GGGUCGGAACCCAAGCUUAGAACdTdT) (SEQ ID NO:88) of the RNA is not shown):

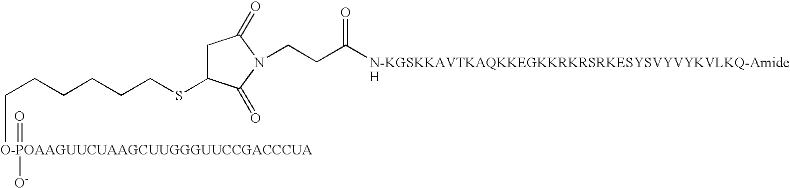

The RNA strand conjugated to the peptide was annealed to its complimentary RNA strand (N168sen). Conjugate 6 was made using the methods and procedures described above in Example 1.

### EXAMPLE 8

### Preparation of Conjugate 7

Preparation of Conjugate 7 (dsCoP857nfR1285): Conjugation of peptide PN857 (H2B 13-48; Asp14 to Glu substitution) (SEQ ID NO:28) and oligonucleotide N169asen (SEQ ID NO:76) having the following structure (complementary sense strand N169sen (GACUCAGCGCUGAGAUCAAUCGGdCdC) (SEQ ID NO:89) of the RNA is not shown):

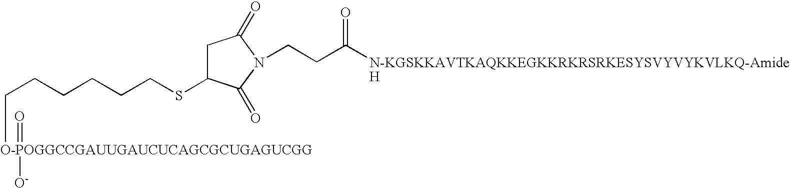

The RNA strand conjugated to the peptide was annealed to its complimentary RNA strand (N169sen). Conjugate 7 was made using the methods and procedures described above in Example 1.

### EXAMPLE 9

### Preparation of Conjugate 8

Preparation of Conjugate 8 (dsCoP857nfR1300): Conjugation of peptide PN857 (H2B 13-48; Asp14 to Glu substitution) (SEQ ID NO:28) and oligonucleotide mTNFα-S1sen (SEQ ID NO:77) having the following structure (complementary sense strand mTNFα-S1asen (GUCUCAGCCUCUUCUCAUUCCUGdCdT) (SEQ ID NO:90) of the RNA is not shown).

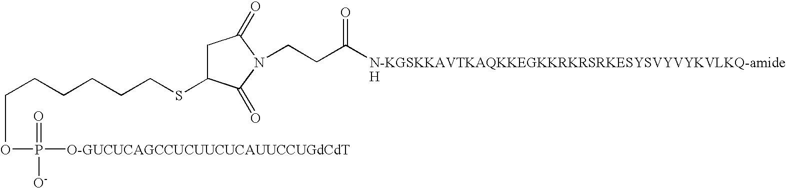

The RNA strand conjugated to the peptide was annealed to its complimentary RNA strand (mTNFα-S1asen). Conjugate 8 was made using the methods and procedures described above in Example 1.

### EXAMPLE 10

### Preparation of Conjugate 9

Preparation of Conjugate 9 (dsCoP857nfR1345): Conjugation of peptide PN857 (H2B 13-48; Asp14 to Glu substitution) (SEQ ID NO:28) and oligonucleotide mTNFα-S1asen (SEQ ID NO:78) having the following structure (complementary sense strand mTNFα-S1sen (GUCUCAGCCUCUUCUCAUUCCUGdCdT) (SEQ ID NO:91) of the RNA is not shown):

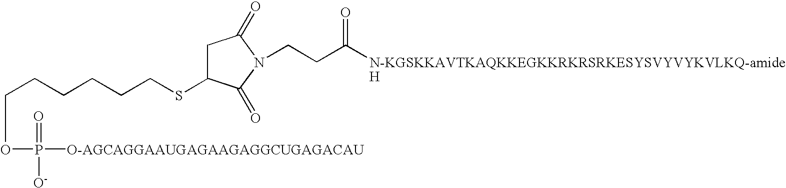

The RNA strand conjugated to the peptide was annealed to its complimentary RNA strand (mTNFα-S1sen). Conjugate 9 was made using the methods and procedures described above in Example 1.

### EXAMPLE 11

### Preparation of Conjugate 10

Preparation of Conjugate 10 (dsCoP872nfR1390): Conjugation of peptide PN872 (H2B 19-36; 2 kDa PEG) (SEQ ID NO:31) and oligonucleotide CN950asen (N163ansen) (SEQ ID NO:68) having the following structure (complementary sense strand N163sen (CCCAUGUGCUCCUCACCCACACCdAT) (SEQ ID NO:92) of the RNA is not shown):

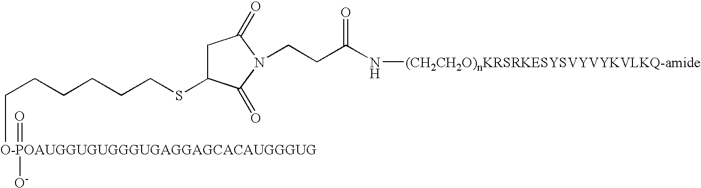

The RNA strand conjugated to the peptide was annealed to its complimentary RNA strand (N163sen). Conjugate 10 was made using the methods and procedures described above in Example 1. PEGylation in the above structure is represented by the formula (CH₂ CH₂O)ₙ.

### EXAMPLE 12

### Preparation of Conjugate 11

Preparation of Conjugate 11 (dsCoP828nfR1265): Conjugation of peptide PN828 (H2B 10-36; Asp14 to Glu substitution) (SEQ ID NO:29) and oligonucleotide CN950asen (N163asen) (SEQ ID NO:68) having the following structure (complementary sense strand N163sen (CCCAUGUGCUCCUCACCCACACCdAT) (SEQ ID NO:93) of the RNA is not shown):

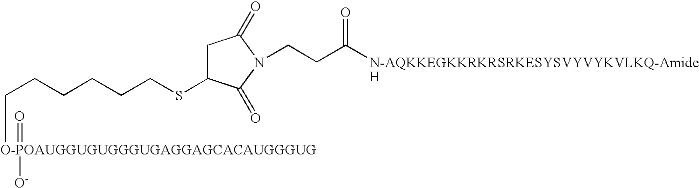

The RNA strand conjugated to the peptide was annealed to its complimentary RNA strand (N163sen). Conjugate 11 was made using the methods and procedures described above in Example 1.

### EXAMPLE 13

### Preparation of Conjugate 12

Preparation of Conjugate 12 (dsCoP750nfR1200): Conjugation of peptide PN750 (H2B 19-36) (SEQ ID NO:30) and oligonucleotide CN950asen (N163ansen) (SEQ ID NO:68) having the following structure (complementary sense strand N163sen (CCCAUGUGCUCCUCACCCACACCdAT) (SEQ ID NO:94) of the RNA is not shown):

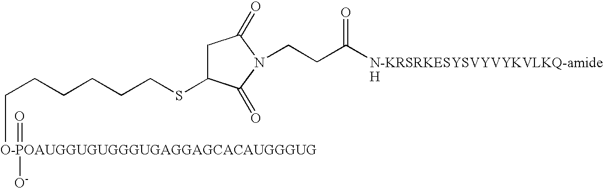

The RNA strand conjugated to the peptide was annealed to its complimentary RNA strand (N163sen). Conjugate 12 was made using the methods and procedures described above in Example 1.

### EXAMPLE 14

### Preparation of Conjugate 13

Preparation of Conjugate 13 (dsCoP751nfR1200): Conjugation of peptide PN751 (H2B 31-36) (SEQ ID NO:33) and oligonucleotide CN950asen (N163ansen) (SEQ ID NO: 68) having the following structure (complementary sense strand N163sen (CCCAUGUGCUCCUCACCCACACCdAT) (SEQ ID NO:95) of the RNA is not shown):

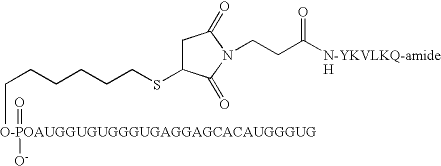

The RNA strand conjugated to the peptide was annealed to its complimentary RNA strand (N163sen). Conjugate 13 was made using the methods and procedures described above in Example 1.

### EXAMPLE 15

### Preparation of Conjugate 14

Preparation of Conjugate 14 (dsCoP963nfR3063): Conjugation of peptide PN963 (H2B 24-36) (SEQ ID NO:32) and oligonucleotide G1498dicerasen (CN3063asen) (SEQ ID NO:72). The RNA strand conjugated to the peptide was annealed to its complimentary RNA strand (G1498dicersen or CN2848sen) (SEQ ID NO: 71). Conjugate 14 has the following structure:

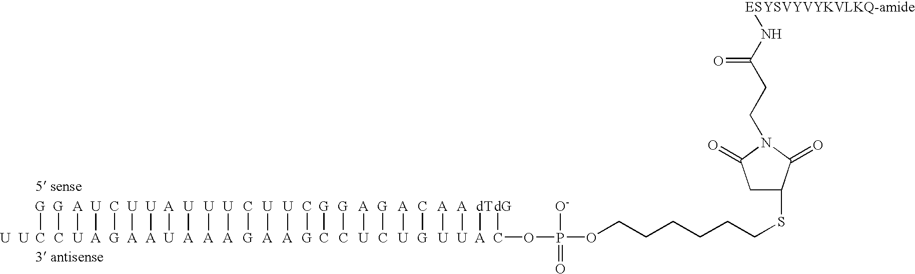

### EXAMPLE 16

### Preparation of Conjugate 15

Preparation of Conjugate 15 (dsCoP3072nfR3063): Conjugation of peptide PN3072 (islet homeodomain) (SEQ ID NO:34) and oligonucleotide G1498dicerasen (CN3063asen) (SEQ ID NO:72). The RNA strand conjugated to the peptide was annealed to its complimentary RNA strand (G1498dicersen or CN2848sen) (SEQ ID NO:71). Conjugate 15 has the following structure:

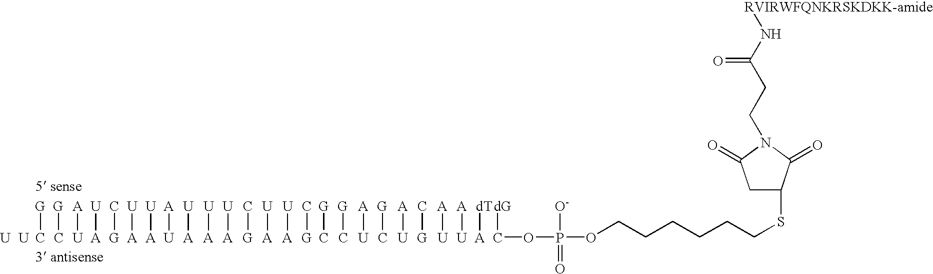

### EXAMPLE 17

### Preparation of Conjugate 16

Preparation of Conjugate 16 (dsCoP3073nfR3063): Conjugation of peptide PN3073 (MPG peptide) (SEQ ID NO:35) and oligonucleotide G1498dicerasen (CN3063asen) (SEQ ID NO:72). The RNA strand conjugated to the peptide was annealed to its complimentary RNA strand (G1498dicersen or CN2848sen) (SEQ ID NO:71). Conjugate 16 has the following structure:

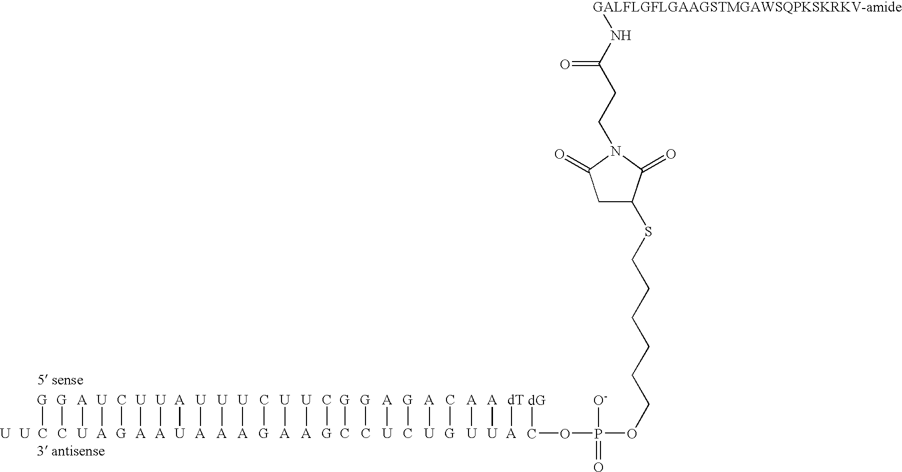

### EXAMPLE 18

### Preparation of Conjugate 17

Preparation of Conjugate 17: Conjugation of peptide PN3070 and the 5′-end of the oligonucleotide G1498dicerasen (CN3063asen).

Oligonucleotide G1498dicerasen is dissolved in 0.5 mL of water to which 2 mL of buffer A (20 mM Tris-HCl, pH 6.8, 50% formamide) is added. Peptide PN3070 is subsequently added which results in the formation of a precipitate that is solubilized by the addition of 1 mL of 2 M TEAA buffer. Upon completion of the reaction, the solvent is removed under reduced pressure and the resulting solid is dissolved in buffer A (20 mM Tris-HCl, pH 6.8, 50% formamide). The material is loaded onto an Amersham Resource Q column and washed with 5 column volumes of buffer A at 6 mL/min. Separation is accomplished by running a linear gradient of buffer B (20 mM Tris-HCl, pH 6.8, 50% formamide, 1 M NaCl) from 15-60% for 15 column volumes at a flow rate of 6 mL/min. The purified conjugate is desalted by slidalyzer dialysis cassettes (10K MWCO) against PBS. The amount of the conjugate is determined spectrophotometrically based on the calculated molar absorption coefficient at λ=260 nm. Purity of the conjugate is confirmed by RP-HPLC.

The sense strand (G1498dicersen) of the RNA peptide conjugate is annealed to its complimentary sense strand in 50 mM potassium acetate, 1 mM magnesium acetate and 15 mM HEPES pH 7.4 by heating at 90° C. for 2 minutes followed by incubation at 37° C. for 1 hour. The formation of the double stranded RNA conjugate is confirmed by non-denaturing (15%) polyacrylamide gel electrophoresis and staining with ethidium bromide.

### EXAMPLE 19

### Preparation of Conjugate 18

Preparation of Conjugate 18: Conjugation of peptide PN3185 and the 5′-end of the oligonucleotide G1498dicerasen (CN3063asen). The RNA strand that is conjugated to the peptide is annealed to its complimentary RNA strand (G1498dicersen). Conjugate 18 is prepared by the methods and procedures described in Example 18.

### EXAMPLE 20

### Preparation of Conjugate 19

Preparation of Conjugate 19: Conjugation of peptide PN3071 and the 5′-end of the oligonucleotide G1498dicerasen (CN3063asen). The RNA strand that is conjugated to the peptide is annealed to its complimentary RNA strand (G1498dicersen). Conjugate 19 is prepared by the methods and procedures described in Example 18.

### EXAMPLE 21

### Preparation of Conjugate 20

Preparation of Conjugate 20: Conjugation of peptide PN3414 and the 5′-end of the oligonucleotide G1498dicerasen (CN3063asen). The RNA strand that is conjugated to the peptide is annealed to its complimentary RNA strand (G1498dicersen). Conjugate 20 is prepared by the methods and procedures described in Example 18.

### EXAMPLE 22

### Preparation of Conjugate 21

Preparation of Conjugate 21: Conjugation of peptide PN3415 and the 5′-end of the oligonucleotide LacZasen. The RNA strand that is conjugated to the peptide is annealed to its complimentary RNA strand (LacZsen). Conjugate 21 is prepared by the methods and procedures described in Example 18.

### EXAMPLE 23

### Preparation of Conjugate 22

Preparation of Conjugate 22: Conjugation of peptide PN3416 and the 5′-end of the oligonucleotide LacZsen. The RNA strand that is conjugated to the peptide is annealed to its complimentary RNA strand (LacZasen). Conjugate 22 is prepared by the methods and procedures described in Example 18.

### EXAMPLE 24

### Preparation of Conjugate 23

Preparation of Conjugate 23: Conjugation of peptide PN3079 and the 5′-end of the oligonucleotide PPIBsen. The RNA strand that is conjugated to the peptide is annealed to its complimentary RNA strand (PPIBasen). Conjugate 23 is prepared by the methods and procedures described in Example 18.

### EXAMPLE 25

### Preparation of Conjugate 24

Preparation of Conjugate 24: Conjugation of peptide PN3671 and the 5′-end of the oligonucleotide PPIBasen. The RNA strand that is conjugated to the peptide is annealed to its complimentary RNA strand (PPIBsen). Conjugate 24 is prepared by the methods and procedures described in Example 18.

### EXAMPLE 26

### Preparation of Conjugate 25

Preparation of Conjugate 25: Conjugation of peptide PN2986 and the 3′-end of the oligonucleotide CN952sen (N163sen). The RNA strand that is conjugated to the peptide is annealed to its complimentary RNA strand (CN950asen). Conjugate 25 is prepared by the methods and procedures described in Example 18.

### EXAMPLE 27

### Preparation of Conjugate 26

Preparation of Conjugate 26: Conjugation of peptide PN740 and the 3′-end of the oligonucleotide CN950asen (N163asen). The RNA strand that is conjugated to the peptide is annealed to its complimentary RNA strand (CN952sen). Conjugate 26 is prepared by the methods and procedures described in Example 18.

### EXAMPLE 28

### Preparation of Conjugate 27

Preparation of Conjugate 27: Conjugation of peptide PN3846 and the 3′-end of the oligonucleotide CN950asen (N163asen). The RNA strand that is conjugated to the peptide is annealed to its complimentary RNA strand (CN952sen). Conjugate 27 is prepared by the methods and procedures described in Example 18.

### EXAMPLE 29

### Preparation of Conjugate 28

Preparation of Conjugate 28: Conjugation of peptide PN3847 and the 3′-end of the oligonucleotide CN740sen (DS1sen). The RNA strand that is conjugated to the peptide is annealed to its complimentary RNA strand (CN741asen). Conjugate 28 is prepared by the methods and procedures described in Example 18.

### EXAMPLE 30

### Preparation of Conjugate 29

Preparation of Conjugate 29: Conjugation of peptide PN3848 and the 3′-end of the oligonucleotide CN741saen (DS1asen). The RNA strand that is conjugated to the peptide is annealed to its complimentary RNA strand (CN740sen). Conjugate 29 is prepared by the methods and procedures described in Example 18.

### EXAMPLE 31

### Preparation of Conjugate 30

Preparation of Conjugate 30: Conjugation of peptide PN3884 and the 3′-end of the oligonucleotide G1498dicersen (2848sen). The RNA strand that is conjugated to the peptide is annealed to its complimentary RNA strand (G1498dicerasen). Conjugate 30 is prepared by the methods and procedures described in Example 18.

### EXAMPLE 32

### Preparation of Conjugate 31

Preparation of Conjugate 31: Conjugation of peptide PN3885 and the 3′-end of the oligonucleotide G1498dicerasen (3063asen). The RNA strand that is conjugated to the peptide is annealed to its complimentary RNA strand (G1498dicersen). Conjugate 31 is prepared by the methods and procedures described in Example 18.

### EXAMPLE 33

### Preparation of Conjugate 32

Preparation of Conjugate 32: Conjugation of peptide PN3886 and the 3′-end of the oligonucleotide N168sen. The RNA strand that is conjugated to the peptide is annealed to its complimentary RNA strand N168asen. Conjugate 32 is prepared by the methods and procedures described in Example 18.

### EXAMPLE 34

### Preparation of Conjugate 33

Preparation of Conjugate 33: Conjugation of peptide PN3948 and the 3′-end of the oligonucleotide N168asen. The RNA strand that is conjugated to the peptide is annealed to its complimentary RNA strand N168sen. Conjugate 33 is prepared by the methods and procedures described in Example 18.

### EXAMPLE 35

### Preparation of Conjugate 34

Preparation of Conjugate 34: Conjugation of peptide PN3980 and the 3′-end of the oligonucleotide N169sen. The RNA strand that is conjugated to the peptide is annealed to its complimentary RNA strand N169asen. Conjugate 34 is prepared by the methods and procedures described in Example 18.

### EXAMPLE 36

### Preparation of Conjugate 35

Preparation of Conjugate 35: Conjugation of peptide PN3980 and the 3′-end of the oligonucleotide N169asen. The RNA strand that is conjugated to the peptide is annealed to its complimentary RNA strand N169sen. Conjugate 35 is prepared by the methods and procedures described in Example 18.

### EXAMPLE 37

### Preparation of Conjugate 36

Preparation of Conjugate 36: Conjugation of peptide PN3981 and the 3′-end of the oligonucleotide mTNFα-S1sen. The RNA strand that is conjugated to the peptide is annealed to its complimentary RNA strand mTNFα-S1asen. Conjugate 36 is prepared by the methods and procedures described in Example 18.

### EXAMPLE 38

### Preparation of Conjugate 37

Preparation of Conjugate 37: Conjugation of peptide PN3982 and the 3′-end of the oligonucleotide mTNFα-S1asen. The RNA strand that is conjugated to the peptide is annealed to its complimentary RNA strand mTNFα-S1sen. Conjugate 37 is prepared by the methods and procedures described in Example 18.

### EXAMPLE 39

### Preparation of Conjugate 38

Preparation of Conjugate 38: Conjugation of peptide PN3983 and the 5′-end of the oligonucleotide G1498dicersen (2848sen). The RNA strand that is conjugated to the peptide is annealed to its complimentary RNA strand G1498dicerasen (3063asen). Conjugate 38 is prepared by the methods and procedures described in Example 18.

### EXAMPLE 40

### Preparation of Conjugate 39

Preparation of Conjugate 39: Conjugation of peptide PN3984 and the 5′-end of the oligonucleotide G1498dicersen (2848sen). The RNA strand that is conjugated to the peptide is annealed to its complimentary RNA strand G1498dicerasen (3063asen). Conjugate 39 is prepared by the methods and procedures described in Example 18.

### EXAMPLE 41

### Preparation of Conjugate 40

Preparation of Conjugate 40: Conjugation of peptide PN3985 and the 5′-end of the oligonucleotide G1498dicersen (2848sen). The RNA strand that is conjugated to the peptide is annealed to its complimentary RNA strand G1498dicerasen (3063asen). Conjugate 40 is prepared by the methods and procedures described in Example 18.

### EXAMPLE 42

### Preparation of Conjugate 41

Preparation of Conjugate 41: Conjugation of peptide PN3846 and the 5′-end of the oligonucleotide G1498dicersen (2848sen). The RNA strand that is conjugated to the peptide is annealed to its complimentary RNA strand G1498dicerasen (3063asen). Conjugate 41 is prepared by the methods and procedures described in Example 18.

### EXAMPLE 43

### Preparation of Conjugate 42

Preparation of Conjugate 42: Conjugation of peptide PN3847 and the 5′-end of the oligonucleotide G1498dicersen (2848sen). The RNA strand that is conjugated to the peptide is annealed to its complimentary RNA strand G1498dicerasen (3063asen). Conjugate 42 is prepared by the methods and procedures described in Example 18.

### EXAMPLE 44

### Preparation of Conjugate 43

Preparation of Conjugate 243: Conjugation of peptide PN3848 and the 5′-end of the oligonucleotide G1498dicersen (2848sen). The RNA strand that is conjugated to the peptide is annealed to its complimentary RNA strand G1498dicerasen (3063asen). Conjugate 43 is prepared by the methods and procedures described in Example 18.

### EXAMPLE 45

### Preparation of Conjugate 44

Preparation of Conjugate 44: Conjugation of peptide PN3884 and the 5′-end of the oligonucleotide G1498dicersen (2848sen). The RNA strand that is conjugated to the peptide is annealed to its complimentary RNA strand G1498dicerasen (3063asen). Conjugate 44 is prepared by the methods and procedures described in Example 18.

### EXAMPLE 46

### Preparation of Conjugate 45

Preparation of Conjugate 45: Conjugation of peptide PN3885 and the 5′-end of the oligonucleotide G1498dicersen (2848sen). The RNA strand that is conjugated to the peptide is annealed to its complimentary RNA strand G1498dicerasen (3063asen). Conjugate 45 is prepared by the methods and procedures described in Example 18.

### EXAMPLE 47

### Preparation of Conjugate 46

Preparation of Conjugate 46: Conjugation of peptide PN3886 and the 5′-end of the oligonucleotide G1498dicersen (2848sen). The RNA strand that is conjugated to the peptide is annealed to its complimentary RNA strand G1498dicerasen (3063asen). Conjugate 46 is prepared by the methods and procedures described in Example 18.

### EXAMPLE 48

### Preparation of Conjugate 47

Preparation of Conjugate 47: Conjugation of peptide PN3948 and the 5′-end of the oligonucleotide G1498dicersen (2848sen). The RNA strand that is conjugated to the peptide is annealed to its complimentary RNA strand G1498dicerasen (3063asen). Conjugate 47 is prepared by the methods and procedures described in Example 18.

### EXAMPLE 49

### Formulations and Physical Characterization of Dicer Substrate Peptide Conjugates and Complexes

Aqueous formulations of dicer substrate peptide conjugates were prepared as shown in Table 6 by dissolving the conjugate in water.

TABLE 6
Aqueous Formulations of Dicer Substrate Peptide Conjugates

| Conjugate Identifier | Concentration |
|---|---|
| Conjugate 14 (dsCoP963nfR3063) | 276 μM/5.1 mg/mL |
| Conjugate 15 (dsCoP3072nfR3063) | 902 μM/17 mg/mL |
| Conjugate 16 (dsCoP3073nfR3063) | 456 μM/8.95 mg/mL |

Formulations of dicer substrate peptide conjugates were also prepared by dissolving the conjugate in water with medium, e.g., OPTI-MEM. Alternatively, formulations of dicer substrate peptide conjugates can be prepared by dissolving the conjugate in water and admixing with a transfection material, e.g., LIPOFECTAMINE RNAiMAX or dsRNA Buffer, which contains RNase-free water, KCl, HEPES free acid, MgCl₂ 6H2O, and KOH, pH of 7.3-7.6. See, e.g., Dharmacon protocol 5×siRNA buffer.

Particles of dicer substrate peptide conjugates were formed in various formulations by condensing the dicer substrate peptide conjugate with a separate charged peptide. For example, particle-containing formulations of dicer substrate peptide conjugates for intracellular delivery were prepared at 1 μM. In a 1.5 ml tube, 196.7 μL water was added, followed by 250 μl of 5 M NaCl, followed by 18.10 μl of 0.51 mg/mL of Conjugate 14 (dsCoP963nfR3063). Finally, 35.17 μL of peptide PN3079 at 0.2 mg/mL was added. This formulation was dialyzed against water with a slidalyzer cassette (MWCO 2000 Da) for two hours. During dialysis, dialysis water was changed four times. The formulation was diluted 10× using water for intracellular delivery. The approximate N/P ratio was two for the dsCoP963nfR3063-PN3079 formulation.

| | |
|---|---|
| PN3079 | |
| RRRRRRRRRR | (SEQ ID NO: 96) |

Particle size and dispersity measurements for some formulations of dicer substrate peptide conjugates and dicer substrate peptide complexes are shown in Table 7. Different rows with identical Sample names indicate multiple measurements for that particular formulation. Conjugates, complexes and delivery peptides alone were compared using different buffers (OPTI-MEM, dsRNA Buffer and 10 mM HEPES, 5% Dextrose, pH 7.4). DX3030 is the unconjugated double stranded G1498 dicer substrate RNA. When conjugated to a delivery peptide, the same RNA is identified as 3063 (e.g., dsCoP963nfR3063 is double stranded G1498 dicer substrate RNA conjugated to the PN963 delivery peptide).

TABLE 7
Formulations of Dicer Substrate Peptide Conjugates and Complexes

| | | | Derived |
|---|---|---|---|
| | Z-Ave | | Count Rate |
| Sample | (d.nm) | PdI | (kcps) |
| OPTI-MEM | 996.5 | 0.853 | 32.1 |
| OPTI-MEM | 251.7 | 0.308 | 33.9 |
| dsRNA Buffer | 0 | 0 | 480.9 |
| PN963 in dsRNA Buffer | 0 | 0 | 26.6 |
| Conjugate 14 (dsCoP963nfR3063) (G stock) in dsRNA Buffer | 127.6 | 1 | 17.4 |
| Conjugate 14 (dsCoP963nfR3063) (G stock) in dsRNA Buffer | 0 | 0 | 17.4 |
| Conjugate 14 (dsCoP963nfR3063) (M stock) in dsRNA Buffer | 163.4 | 1 | 22.3 |
| Conjugate 14 (dsCoP963nfR3063) (M stock) in dsRNA Buffer | 500.7 | 0.512 | 22.1 |
| Conjugate 15 (dsCoP3072nfR3063) in dsRNA Buffer | 268.9 | 0.73 | 46.8 |
| Conjugate 15 (dsCoP3072nfR3063) in dsRNA Buffer | 279.1 | 0.46 | 45.6 |
| Conjugate 15 (dsCoP3072nfR3063) in dsRNA Buffer | 128.3 | 0.295 | 60.9 |
| Conjugate 16 (dsCoP3073nfR3063) in dsRNA Buffer | 96.84 | 0.4 | 58.4 |
| Conjugate 14 (dsCoP963nfR3063) in 10 mM HEPES, 5% Dextrose, pH 7.4 | 8.002 | 0.48 | 114.5 |
| Conjugate 14 (dsCoP963nfR3063) in 10 mM HEPES, 5% Dextrose, pH 7.4 | 8.71 | 0.518 | 108.8 |
| DX3030-PN963 Complex in dsRNA Buffer | 571.3 | 0.31 | 135.2 |
| DX3030-PN963 Complex in dsRNA Buffer | 1712 | 1 | 124.9 |
| Conjugate 14 (dsCoP963nfR3063)-PN3079 Complex in 10 mM HEPES, | 2279 | 0.91 | 443.6 |
| 5% Dextrose, pH 7.4 | | | |
| Conjugate 14 (dsCoP963nfR3063)-PN3079 Complex in 10 mM HEPES, | 1367 | 0.626 | 383.2 |
| 5% Dextrose, pH 7.4 | | | |
| Conjugate 14 (dsCoP963nfR3063)-PN3079 Complex in 10 mM HEPES, | 1105 | 0.669 | 186.9 |
| 5% Dextrose, pH 7.4 | | | |
| Conjugate 14 (dsCoP963nfR3063)-PN3079 Complex in 10 mM HEPES, | 1467 | 0.556 | 344.1 |
| 5% Dextrose, pH 7.4 | | | |
| Before Dialysis Conjugate 14 (dsCoP963nfR3063)-PN3079 Complex in | 7.513 | 0.45 | 35.4 |
| HEPES/Dextrose 2.5 M NaCl | | | |
| Before Dialysis Conjugate 14 (dsCoP963nfR3063)-PN3079 Complex in | 6.358 | 0.396 | 33.9 |
| HEPES/Dextrose 2.5 M NaCl | | | |
| PN963 in OPTI-MEM 10 nM | 317.8 | 0.374 | 34.5 |
| PN963 in OPTI-MEM 10 nM | 109.2 | 0.284 | 32.1 |
| Conjugate 14 (dsCoP963nfR3063) (G stock) in OPTI-MEM 10 nM | 238.2 | 0.295 | 45.6 |
| Conjugate 14 (dsCoP963nfR3063) (G stock) in OPTI-MEM 10 nM | 44.36 | 1 | 42.4 |
| Conjugate 14 (dsCoP963nfR3063) (M stock) in OPTI-MEM 10 nM | 167.2 | 0.267 | 31.3 |
| Conjugate 14 (dsCoP963nfR3063) (M stock) in OPTI-MEM 10 nM | 69.39 | 0.262 | 30.1 |
| Conjugate 15 (dsCoP3072nfR3063) in OPTI-MEM 10 nM | 487.6 | 0.54 | 34.3 |
| Conjugate 15 (dsCoP3072nfR3063) in OPTI-MEM 10 nM | 183.2 | 0.718 | 34.8 |
| Conjugate 16 (dsCoP3073nfR3063) in OPTI-MEM 10 nM | 372 | 0.427 | 45 |
| Conjugate 16 (dsCoP3073nfR3063) in OPTI-MEM 10 nM | 137.5 | 0.227 | 42.9 |
| Conjugate 14 (dsCoP963nfR3063) conjugate in OPTI-MEM 10 nM | 249 | 0.306 | 37.8 |
| Conjugate 14 (dsCoP963nfR3063) in OPTI-MEM 10 nM | 190.9 | 0.359 | 37 |
| DX3030 PN963 complex in OPTI-MEM 10 nM | 420.5 | 0.404 | 28.5 |
| DX3030 PN963 complex in OPTI-MEM 10 nM | 18.6 | 0.776 | 28.1 |
| Conjugate 14 (dsCoP963nfR3063)-PN3079 Complex in OPTI-MEM 10 nM | 413.2 | 0.472 | 34.7 |
| Conjugate 14 (dsCoP963nfR3063)-PN3079 Complex in OPTI-MEM 10 nM | 737.4 | 0.686 | 15.7 |
| DX3030-PN963 Complex in HEPES/Dextrose into OPTI-MEM 10 nM | 225.1 | 0.277 | 30.4 |
| DX3030-PN963 Complex in HEPES/Dextrose into OPTI-MEM 10 nM | 54.03 | 0.468 | 30.1 |

The data in Table 7 show that dicer substrate peptide conjugates, in buffer either alone or with dextrose, tend to remain molecularly disperse as shown by the low count rates. Particles of a dicer substrate peptide conjugate can be formed in solution by admixing with PN3079.

The zeta potentials for some of these dicer substrate peptide conjugate and complex formulations are shown in Table 8.

TABLE 8
Zeta Potentials for Dicer Substrate Peptide Conjugate Formulations

| | zeta |
|---|---|
| | potential |
| Sample | (mv) |
| Conjugate 14 (dsCoP963nfR3063) in 10 mM HEPES, | −3.17 |
| 5% Dextrose, pH 7.4 | |
| Conjugate 14 (dsCoP963nfR3063) in 10 mM HEPES, | −2.53 |
| 5% Dextrose, pH 7.4 | |
| DX3030-PN963 Complex in dsRNA Buffer | −3.78 |
| DX3030-PN963 Complex in dsRNA Buffer | −1.57 |
| Conjugate 14 (dsCoP963nfR3063)-PN3079 Complex in | −14.8 |
| 10 mM HEPES, 5% Dextrose, pH 7.4 | |
| Conjugate 14 (dsCoP963nfR3063)-PN3079 in 10 mM | −15.5 |
| HEPES, 5% Dextrose, pH 7.4 | |
| DX3030-PN963 Complex in HEPES, 5% Dextrose, pH 7.4 | −8.33 |
| DX3030-PN963 Complex in HEPES, 5% Dextrose, pH 7.4 | −24.7 |
| 10 mM HEPES, 5% Dextrose, pH 7.4 | −0.783 |

### EXAMPLE 50

### Dicer Substrate RNAs Screened for hTNF-α Knockdown Activity

The present example illustrates dicer substrate RNAs screened for effective reduction of hTNF-α gene expression levels.

The significance of targeting the hTNF-α gene is that it is implicated in mediating the occurrence or progression of rheumatoid arthritis (RA) when over-expressed in human and other mammalian subjects. Therefore, targeted reduction of hTNF-α gene expression can be used as a treatment for RA.

The dsRNAs YC12 and N161 through N164 were screened for sequence-specific post-transcriptional gene silencing of the hTNF-αgene. The sense (top) and antisense (bottom) strands of YC12, N161, N162 and N164 range in length from 21 to 27 nucleotides and are shown in Table 9 (the nucleotide sequence of the sense and antisense strands of dicer substrate dsRNA N163 are shown in Table 5). Silencing of the hTNF-α gene was determined by transfecting the individual dsRNAs YC12 and N161-N164 with LIPOFECTAMINE 2000 (Invitrogen) into LPS stimulated human monocytes. Qneg (QUIAGEN) is a random nucleic acid sequence and functioned as a dsRNA negative control.

TABLE 9

siRNAs Targeted to hTNF-α

| | |
|---|---|
| dsRNA | Nucleic Acid Sequence |

| | |
|---|---|
| YC12 | GCCUGUACCUCAUCUACUCUU |
| | (SEQ ID NO: 97) |
| | GAGUAGAUGAGGUACAGGCUU |
| | (SEQ ID NO: 98 |
| N161 | GCCUCUUCUCCUUCCUGAUCGUGdGdC |
| (dicer substrate) | (SEQ ID NO: 99) |
| | GCCACGAUCAGGAAGGAGAAGAGGCUG |
| | (SEQ ID NO: 100) |
| N162 | GCCUGCUGCACUUUGGAGUGAUCdGdG |
| (dicer substrate) | (SEQ ID NO: 101) |
| | CCGAUCACUCCAAAGUGCAGCAGGCAG |
| | (SEQ ID NO: 102) |
| N164 | ACCUCAUCUACUCCCAGGUCCUCdTdT |
| (dicer substrate) | (SEQ ID NO: 103) |
| | AAGAGGACCUGGGAGUAGAUGAGGUAC |
| | (SEQ ID NO: 104) |
| Qneg | UUCUCCGAACGUGUCACGUTT |
| (negative control) | (SEQ ID NO: 105) |
| | ACGUGACACGUUCGGAGAATT |
| | (SEQ ID NO: 106) |

Briefly, human monocytes were isolated from fresh human blood samples, from healthy donors, purchased from Golden West Biologicals (Temecula, Calif.). Blood samples were diluted with PBS at a 1:1 ratio immediately after receiving. Peripheral blood mononuclear cells (PBMC) were first isolated by Ficoll (Amersham, Piscataway, N.J.) gradient from whole blood. Monocytes were further purified from PBMCs using Miltenyi CD14 positive selection kit (MILTENYI BIOTEC GmbH, Germany) following the manufacturer's instructions. The purity of the monocyte preparation was greater than 95% as judged by flow cytometry after staining cells with anti-CD14 antibody (BD Biosciences, San Jose, Calif.). Isolated human monocytes were maintained in Iscove's modified Dulbecco's medium (IMDM) with 4 mM L-glutamine, nonessential amino acids and 10% fetal bovine serum. Purified human monocytes were maintained overnight in complete media (described above) before induction and knockdown assays. All cells were cultured at 37° C. and 5% CO₂ supplemented with an antibiotic mixture containing 100 units/ml penicillin, 100 ug/ml streptomycin and 0.25 mg/ml Fungizone (INVITROGEN).

Human monocytes were activated by adding 0.1-1.0 ng/ml of Liposaccharides (LPS; Sigma, St Louis, Mo.) to the cell culture to stimulate tumor necrosis factor-α (TNF-α) production. Cells were harvested three hours after incubation with LPS and mRNA levels were determined by QUANTIGENE assay (GENOSPECTRA) according to the manufacturer's instructions. Post-induction changes in TNF-α levels were determined by ELISA (BD Biosciences, San Jose, Calif.), following the manufacturer's protocol.

Monocytes were seeded in a 96 well plate with OptiMEM (Invitrogen, Carlsbad, USA) at 100,000 cells/100 μl/well. For cells transfected with LIPOFECTAMINE 2000 (INVITROGEN), the manufacturer's protocol was followed. All transfections were carried out for three hours at 37° C. and 5% CO₂. After the three hour incubation, the transfection media was replaced with complete media and the cells were allowed to recover overnight. bDNA assays (QUANTIGENE assay from GENOSPECTRA) were performed following the manufacturer's protocol. Media samples used for the ELISA assay were taken from the cell culture directly. hTNF-α plasma levels were determined by ELISA by diluting samples 1:2 according to the manufacturer's protocol (R&D SYSTEMS).

Each of YC12 and N161-N164 were transfected with LIPOFECTAMINE 2000 at 0.16 nM, 0.8 nM, 4 nM and 20 nM concentrations (no delivery peptide) into LPS activated human monocytes. Qneg dsRNA served as a negative control and has the following nucleic acid sequence:

| | |
|---|---|
| G1498 (non-dicer) sen | |
| GGAUCUUAUUUCUUCGGAGdTdT | (SEQ ID NO: 107) |
| G1498 (non-dicer) asen | |
| CUCCGAAGAAAUAAGAUCCTT | (SEQ ID NO: 108) |

Table 10 summarizes the TNF-α knockdown activity for each dsRNA. The observed Qneg knockdown activity was normalized to 100% gene expression levels, and the knockdown activity for each dsRNA was presented as a relative percentage of the Qneg negative control.

TABLE 10
TNF-α Knockdown Activity of Lipofectamine Transfected dsRNA

| | dsRNA | % TNF-α mRNA |
|---|---|---|
| dsRNA | Concentration | Expression Level |
| Qneg | 0.16 nM | 100% |
| (Negative Control) | 0.8 nM | |
| | 4 nM | |
| | 20 nM | |
| YC12 | 0.16 nM | 70% |
| | 0.8 nM | 83% |
| | 4 nM | 66% |
| | 20 nM | 82% |
| N161 | 0.16 nM | 100% |
| | 0.8 nM | 99% |
| | 4 nM | 110% |
| | 20 nM | 123% |
| N162 | 0.16 nM | 89% |
| | 0.8 nM | 81% |
| | 4 nM | 91% |
| | 20 nM | 112% |
| N163 | 0.16 nM | 76% |
| | 0.8 nM | 66% |
| | 4 nM | 65% |
| | 20 nM | 57% |
| N164 | 0.16 nM | 86% |
| | 0.8 nM | 83% |
| | 4 nM | 71% |
| | 20 nM | 76% |

The data in Table 10 show that dicer substrates N163 and YC12 effectively reduced hTNF-α mRNA levels in human monocytes. The data in Table 10 show that the dsRNAs N161, N162 and N164 did not have a significant effect on TNF-α mRNA levels when compared to the Qneg siRNA negative control. In contrast, the dsRNA Y12 reduced TNF-α mRNA levels to 66% of the Qneg negative control while the dsRNA N163 reduced mRNA levels to 57% of the Qneg negative control.

### EXAMPLE 51

### dsRNA Peptide Conjugates Reduced hTNF-α mRNA Levels in Human Monocytes

This example shows that dicer substrates conjugated to a delivery peptide effectively reduced the expression of hTNF-α mRNA levels in human monocytes. A summary of the dsRNA peptide conjugate knockdown activity is shown below in Table 11. The observed Qneg knockdown activity was normalized to 100% gene expression levels, and the knockdown activity for each dicer substrate peptide conjugate was presented as a relative percentage of the Qneg negative control.

Conjugate 1 (dsCoP277nfR950), Conjugate 2 (dsCoP277nfR952) and a Qneg control conjugate (delivery peptide PN277 conjugate to the 5′-end of the Qneg sense strand of the dsRNA Qneg) were incubated with human monocytes at 1 nM, 10 nM, 100 nM and 200 nM concentrations. The TNF-α knockdown activity for each conjugate is shown in Table 11.

TABLE 11
RNA Dicer Substrate Polypeptide Conjugate TNF-α Knockdown Activity

| | dsRNA Peptide | % TNF-α |
|---|---|---|
| | Conjugate | mRNA Expression |
| dsRNA/Polypeptide Conjugate | Concentration | Level |
| Qneg/PN277 | 1 nM | 100% |
| (negative control) | 10 nM | |
| | 100 nM | |
| | 200 nM | |
| Conjugate 1 (dsCoP277nfR950) | 1 nM | 120% |
| | 10 nM | 95% |
| | 100 nM | 73% |
| | 200 nM | 38% |
| Conjugate 2 | 1 nM | 80% |
| (dsCoP277nfR952) | 10 nM | 78% |
| | 100 nM | 62% |
| | 200 nM | 68% |

The data in Table 11 shows that dicer substrate N163 conjugated to the PN277 delivery peptide effectively reduced hTNF-α mRNA expression levels regardless whether the peptide was covalently linked to the 5′-end of the sense strand or the 5′-end of the antisense strand of the N163 dsRNA molecule. More specifically, the dsRNA peptide Conjugate 2 (dsCoP277nfR952) reduced hTNF-α mRNA levels to 62% of the Qneg peptide conjugate negative control mRNA levels, while Conjugate 1 (dsCoP277nfR950) reduced hTNF-α mRNA levels to 38% of Qneg peptide conjugate negative control mRNA levels.

The data in Table 11 shows that dicer substrate peptide conjugates of the present disclosure effectively reduced hTNF-α mRNA levels.

### EXAMPLE 52

### Processing of dsRNA Peptide Conjugates by Dicer Endonuclease

This example demonstrates that dsRNAs conjugated to a delivery peptide are processed by dicer endonuclease (RNase III).

The purpose of this Example was to determine whether a dicer substrate RNA conjugated to a delivery peptide remained a target for dicer endonuclease or whether the conjugated delivery peptide would interfere with dicer processing of the dsRNA.

In this example, three dsRNA peptide conjugates (Qneg conjugated to PN277, dsCoP277nfR950 and dsCoP277nfR952) were incubated in the presence or absence of dicer endonuclease. In addition, the nonconjugated N163 dsRNA was incubated in the presence or absence of the dicer endonuclease.

Digestion by dicer endonuclease and analysis by LC-MS was carried out as follows: dsRNA and dsRNA peptide conjugates were incubated with Dicer endonuclease (Stratagene). Digestion was performed in a total volume of 10 μL, and allowed to incubate overnight at 37° C. Following the overnight incubation, a 2 μL sample of the digestion mixture was mixed with 2× loading dye and analyzed by gel electrophoresis on a 15% TBE with Urea and a 15% TBE non-denaturing polyacrylamide gel.

LC-MS was run using an XTerra C18 column, 2.5 μm, 2.1×50 mm (Waters) held at 65° C. The mobile phase was 100 mM hexafluoroisopropanol, 7 mM triethylamine, and elution with 100% methanol. The gradient was 5-16% over 40 min. The eluent was split into a PDA and a Waters Micromass ZQ ESI single-quad mass spectrometer run in negative ion mode. The capillary voltage was 3.0 kV and the cone voltage was 45V. Desolvation took place at 300° C., assisted with 600 L/hr N2. The source was held at 90° C. The acquisition scan rate was 1000-2000 m/z over 1 sec.

A comparison by polyacrylamide gel electrophoresis showed that both the non-dicer incubated and dicer incubated Qneg dsRNA/polypeptide conjugate migrated as “sharp” bands of the same molecular weight, which, as expected, indicated that the Qneg dsRNA/polypeptide conjugate was not processed by dicer endonuclease. N163 dsRNA (no delivery peptide) in the absence of the dicer endonuclease migrated as a “sharp” band on the polyacrylamid gel. However, a slightly shorter N163 dsRNA duplex was observed when it was incubated with dicer as evidenced by a slightly smaller molecular weight band compared to the non-incubated N163 dsRNA. Differential dicer processing was observed between Conjugate 1 (dsCoP277nfR950) where the 5′-end of the antisense strand of the N163 dsRNA molecule was covalently linked to the delivery peptide and Conjugate 2 (dsCoP277nfR952) where the 5′-end of the sense strand of the N163 dsRNA molecules was covalently linked to the delivery peptide. In the absence of dicer endonuclease, both these dicer substrate delivery peptide conjugates migrated as distinct bands of equivalent molecular weight, indicating no dicer processing, as expected. However, in the presence of the dicer endonuclease, for Conjugate 2 (dsCoP277nfR952; sense strand linkage) two different molecular weight bands were observed. The molecular weight of one band was equivalent to the non-processed conjugate and the second band had a molecular weight equivalent to the dicer processed N163 dsRNA (no polypeptide), which indicated that the delivery peptide was no longer covalently linked to the N163 dsRNA. The intensity of the higher molecular weight band (i.e., the non-processed N163 dsRNA delivery peptide conjugate) was approximately 2-fold more intense than the lower molecular weight band (i.e., processed N163 dsRNA delivery peptide), which indicated that a majority of Conjugate 2 (dsCoP277nfR952; sense strand linkage) was not processed. In contrast, Conjugate 1 (dsCoP277nfR950; anti-sense strand linkage) exhibited greater susceptibility to dicer processing as evidenced by a relatively faint band equivalent in size to the non-processed conjugate and a more intense molecular weight band equivalent in size to the dicer processed N163 dsRNA (no polypeptide). The relatively higher level of susceptibility to dicer processing for Conjugate 1 (dsCoP277nfR950) compared to Conjugate 2 (dsCoP277nfR952) correlates with the greater knockdown activity observed for Conjugate 1 (dsCoP277nfR950).

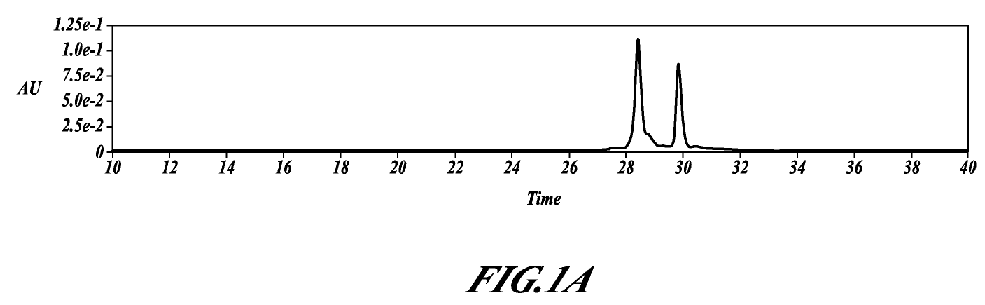

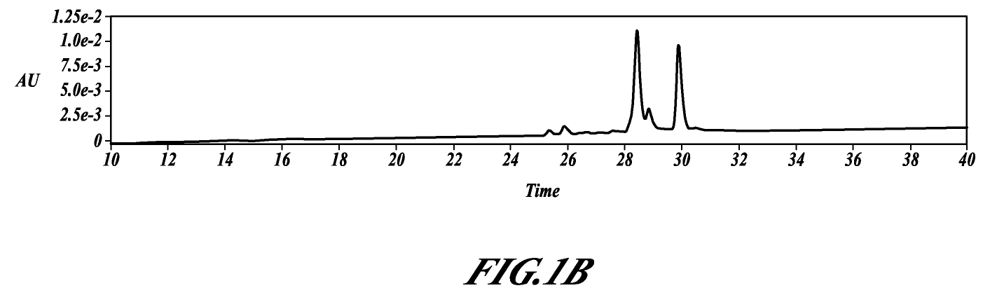

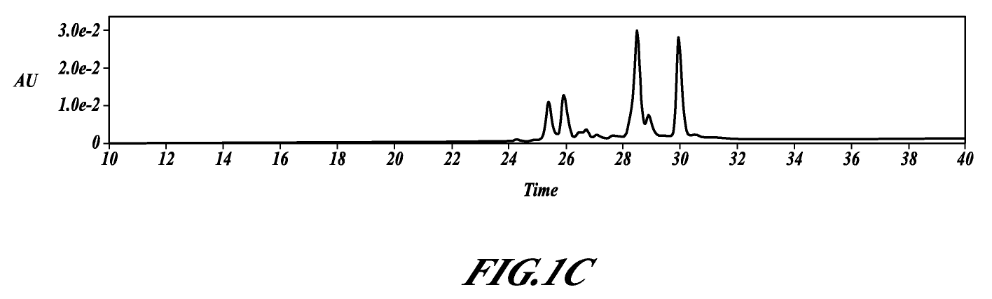

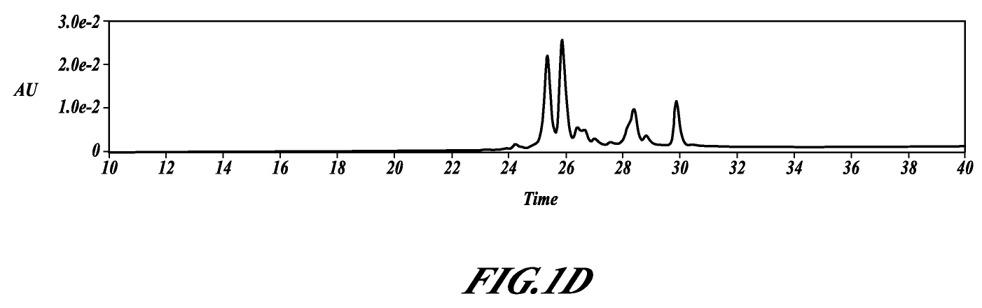

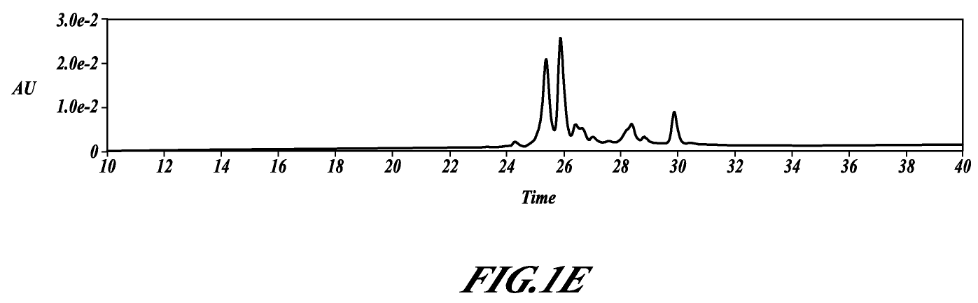

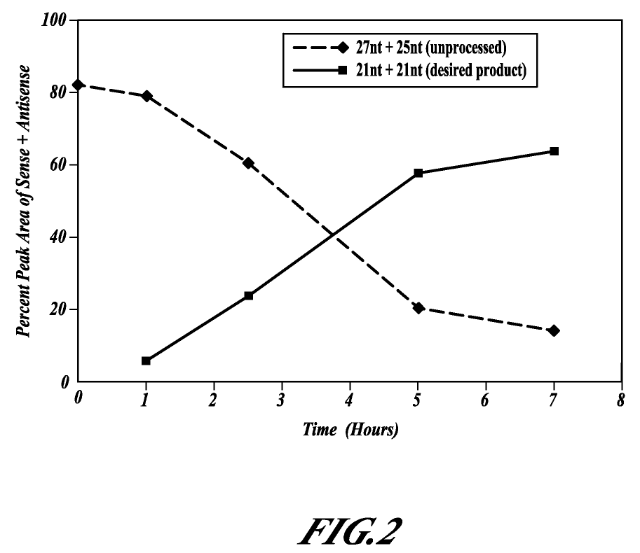

Processing of N163 dsRNA by dicer endonuclease resulted in 21-mer RNAs. FIG. 1 shows the RP-HPLC analysis of dicer endonuclease processing kinetics for non-conjugated N163 dsRNA. FIG. 1(A) shows the RP-HPLC for unprocessed N163 dsRNA. FIGS. 1(B-E) show the RP-HPLC for N163 dsRNA incubated with dicer endonuclease for (B) 1 hr, (C) 2.5 hr, (D) 5 hr, and (E) 7 hr. These data are shown in the chart in FIG. 2.

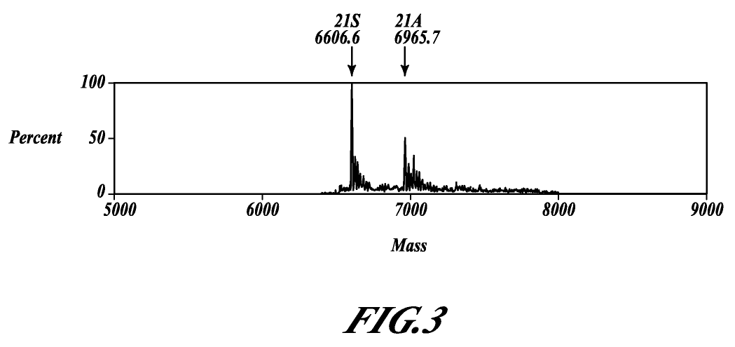

The identity of RNAs obtained after 7 hours digestion of N163 dsRNA by dicer endonuclease that are shown in FIG. 1(E) was confirmed by ESI-MS analysis as shown in FIG. 3. FIG. 3 shows peaks at mass 6606.6 corresponding to the 21-mer sense strand dicer cleavage product of N163 dsRNA, and mass 6965.7 corresponding to the 21-mer antisense strand dicer cleavage product of N163 dsRNA.

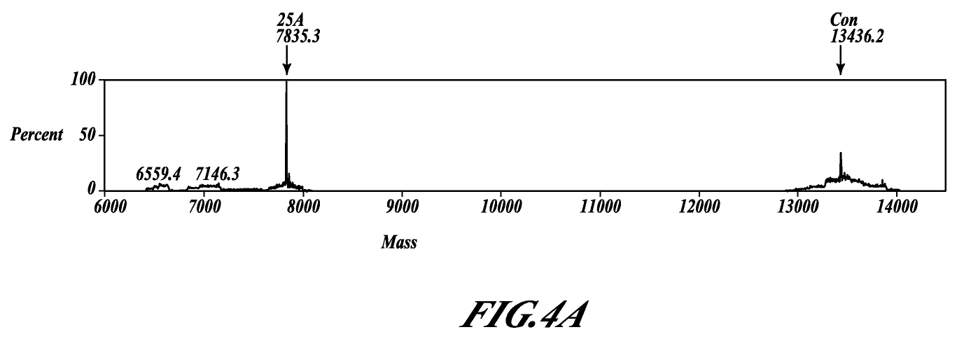

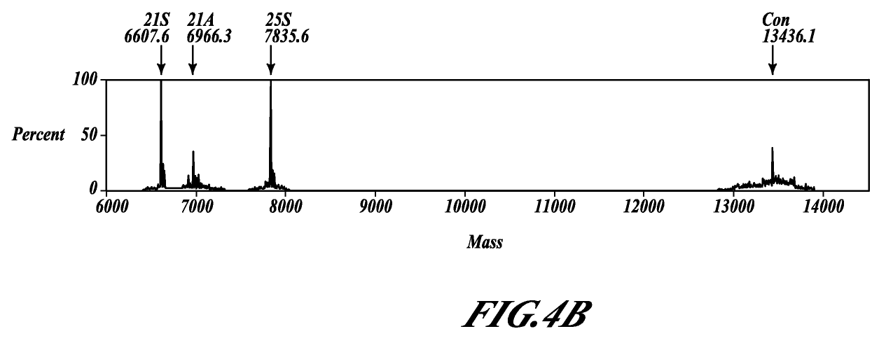

The identity of RNAs after digestion of conjugated N163 dsRNA by dicer endonuclease was confirmed by ESI-MS analysis as shown in FIG. 4. FIG. 4 shows the ESI-MS of dicer endonuclease processing for Conjugate 5 (dsCoP857nfR1280; delivery peptide PN857 conjugated to N163 dsRNA via the 5′-end of N163asen).

In FIG. 4(A), the control incubation of Conjugate 5 (dsCoP857nfR1280) without dicer endonuclease present was obtained. FIG. 4(A) shows peaks at mass 13436.2 corresponding to the conjugate of 27-nt antisense strand of the N163 dsRNA with delivery peptide PN857, and at mass 7835.3 corresponding to the 25-nt sense strand of the N163 dsRNA.

In FIG. 4(B), incubation of Conjugate 5 (dsCoP857nfR1280) with dicer endonuclease present for 8 hours was obtained. FIG. 4(B) shows peaks at mass 13436.1, corresponding to the 27-nt antisense strand conjugate of the N163 dsRNA with delivery peptide PN857, at mass 7835.6, corresponding to the 25-nt sense strand of the N163 dsRNA, at mass 6966.3, corresponding to the 21-mer antisense strand dicer cleavage product of the conjugated 27-mer of the N163 dsRNA, and at mass 6607.6, corresponding to the 21-mer sense strand dicer cleavage product of the 25-mer of the N163 dsRNA.

### EXAMPLE 53

### Dual Reporter Luciferase Assay for Viral mRNA Knockdown

Viral mRNA knockdown in vitro for dicer substrate peptide conjugates was determined using a dual reporter luciferase assay. Assay was performed with A549 cells and/or Vero cells.

For transfection in A549 cells, cells were seeded at 1.2×10⁴ cells/well in a 96-well flat bottom plate one day before transfection in 100 μL 10% FBS, DMEM media per well. Each dsRNA or dicer substrate peptide conjugate and pSiCheck plasmid carrying the influenza viral genome segment at 100 ng/μL were complexed with LIPOFECTAMINE 2000 (Invitrogen) and incubated for 20 minutes at room temperature in 25 μL OPTIMEM (total volume)(GIBCO). A549 cells were washed with OPTI-MEM, and 25 μL of the transfection complex in OPTI-MEM was then added to each well. Triplicate wells were tested for each condition. An additional control well with no transfection condition was prepared. For transfection with Conjugate 15 (dsCoP3072nfR3063), five hours post-transfection, the media was removed and 75 μL of fresh serum containing media was added to each well. The plate was incubated at 37° C., 5% CO₂, for 24 hours.

See Example 23 for generally transfection conditions for Vero cells.

Supernatants from each well were tested in a DUAL GLO luciferase assay (PROMEGA) in cells using a construct in which a first Renilla luciferase test reporter was fused to an influenza viral gene so that it provided a signal inversely proportional to the RISC-based activity of a co-transfected dicer substrate in knocking down the influenza viral mRNA. A second reporter in the construct was set to firefly luciferase so that a ratio between the signal for the first reporter and the second reporter would remove non-specific effects from the measured dicer substrate activity and differences in transfection efficiency.

Conjugate 15 (dsCoP3072nfR3063) was transfected at 10 nM, 1 nM, 0.1 nM, 0.01 nM, and 0.001 nM. Viral mRNA knockdown in A549 cells for the influenza-specific dicer substrate peptide Conjugate 15 (dsCoP3072nfR3063) and DX3030 (the 3063 dicer substrate absent the conjugated delivery peptide) is shown in Table 12 as a percent relative to dicer scrambled negative control DX3039. DX3039 is a dicer substrate scrambled negative control having a sense and antisense strand with the following nucleic acid sequence:

| | |
|---|---|
| DX3039 Sense Strand | |
| CUUCCUCUCUUUCUCUCCCUUGUdGdA | (SEQ ID NO: 109) |
| DX3039 Antisense Strand | |
| UCACAAGGGAGAGAAAGAGAGGAAGGA | (SEQ ID NO: 110) |

TABLE 12
Dicer Substrate Peptide Conjugate Viral mRNA
Knockdown in A549 Cells

Treatment

| | Conjugate 15 | DX3030 |
|---|---|---|
| Concentration | (dsCoP3072nfR3063) | (no delivery peptide) |
| 10 nM | 88.15% | 81.26% |
| 1 nM | 42.89% | 88.72% |
| 0.1 nM | 15.18% | 79.84% |
| 0.01 nM | 9.02% | 50.83% |
| 0.001 nM | 1.77% | 13.49% |

The data in Table 12 demonstrate that dicer substrate peptide Conjugate 15 (dsCoP3072nfR3063) reduced viral mRNA levels in vitro by over 88% at 10 nM and over 42% at 1 nM compared to the negative control.

Conjugate 14 (dsCoP963nfR3063) was transfected at 500 nM, 250 nM, 100 nM and 10 nM in A549 cells and also in Vero Cells. Four different dosing models were performed. Mode A involved transfecting cells with Conjugate 14 for three hours followed by transfection of the luciferase reporter plasmid with LIPOFECTAMINE 2000 for three hours. Mode B involved transfecting cells with the luciferase reporter plasmid with LIPOFECTAMINE 2000 for three hours followed by transfection with Conjugate 14 for three hours. Mode C involved co-transfection with Conjugate 14 and the luciferase reporter plasmid with LIPOFECTAMINE 2000 for five hours. Finally, Mode D, involved transfecting cells with the luciferase reporter plasmid with LIPOFECTAMINE 2000 for three hours followed by transfection with Conjugate 14 for three hours followed by transduction with WNS virus at MOI of 0.01 for one hour.

Viral mRNA knockdown in A549 cells for the influenza-specific dicer substrate peptide Conjugate 14 (dsCoP963nfR3063) and DX3030 (the 3063 dicer substrate absent the conjugated delivery peptide) is shown in Table 13 as a percent relative to dicer scrambled negative control DX3148. Viral mRNA knockdown in Vero cells is shown in Table 14. The term “L2K” in Table 13 refers to LIPOFECTAMINE 2000. dsCoP963nfRQNC is the Qneg RNA conjugated to the delivery peptide PN963.

TABLE 13
Dicer Substrate Peptide Conjugate Viral mRNA Knockdown in A549 Cells

| | | DX3148 | DX3030 | | Conjugate 14 | |
|---|---|---|---|---|---|---|
| | | with | with | | (dsCoP963nfR3063) | Conjugate 14 |
| Mode | Conc. | L2K | L2K | dsCoP963nfRQNC | with L2K | (dsCoP963nfR3063) |
| A | 500 nM | 0% | 80% | 0% | 57.9% | 70% |
| | 250 nM | | | | | 50.8% |
| | 100 nM | | 60% | | 15.6% | 16.5% |
| | 10 nM | | | | | |
| B | 500 nM | 0% | 69.6% | 0% | 67.3% | 72.4% |
| | 250 nM | | | | | 72.5% |
| | 100 nM | | 70.3% | | 64.5% | 67.6% |
| | 10 nM | | | | | 54.9% |
| C | 500 nM | 18.8% | 75.6% | 49.2% | 6.1% | 41.7% |
| | 250 nM | | | | | 72.8% |
| | 100 nM | 0% | 57.5% | 0% | 69.3% | 74.4% |
| | 10 nM | | | | | 90% |
| D | 500 nM | 22% | 86.8% | 19% | 76.3% | 73.5% |
| | 250 nM | | | | | 65% |
| | 100 nM | 20.5% | 71.2% | 17% | 41.9% | 54.2% |
| | 10 nM | | | | | 33% |

TABLE 14
Dicer Substrate Peptide Conjugate Viral mRNA Knockdown in Vero Cells

| | | DX3148 | DX3030 | | Conjugate 14 | |
|---|---|---|---|---|---|---|
| | | with | with | | (dsCoP963nfR3063) | Conjugate 14 |
| Mode | Conc. | L2K | L2K | dsCoP963nfRQNC | with L2K | (dsCoP963nfR3063) |
| A | 500 nM | 0% | 56.7% | 0% | 49.4% | 52.1% |
| | 250 nM | | | | | 53.5% |
| | 100 nM | | 48.5% | | 25.1% | 1.1% |
| | 10 nM | | | | | |
| B | 500 nM | 0% | 43.2% | 0% | 39.8% | 56.8% |
| | 250 nM | | | | | 52.6% |
| | 100 nM | | 53% | | 42% | 44.2% |
| | 10 nM | | | | | 40.8% |
| C | 500 nM | 0% | 24.1% | 0% | 0% | 13.2% |
| | 250 nM | | | | | |
| | 100 nM | | 59.3% | | 32.8% | 59.7% |
| | 10 nM | | | | | 79.2% |
| D | 500 nM | 0% | 58.5% | 0% | 31.8% | 28.5% |
| | 250 nM | | | | | 14.6% |
| | 100 nM | | 23.9% | | 7.7% | 1.6% |
| | 10 nM | | | | | |

The data in Tables 14 and 15 demonstrate that dicer peptide conjugate Conjugate 14 (dsCoP963nfR3063) reduced viral mRNA levels in both A549 and Vero cells in a dose dependent manner.

### EXAMPLE 54

### Viral Titer Reduction in Vero Cells by Influenza-Specific Dicer Substrate Peptide Conjugates

Reduction of WSN influenza viral titer in Vero cells by a dicer substrate peptide conjugate was determined. Vero cells were seeded at 6.5×10⁴ cells/well the day before transfection in 500 μl 10% FBS/DMEM media per well. Samples of 10, 1, 0.1, and 0.01 nM stock of each dsRNA or dicer substrate peptide conjugate were complexed with 1.0 μl (1 mg/mL stock) of LIPOFECTAMINE 2000 (Invitrogen) and incubated for 20 minutes at room temperature in 150 μL OPTIMEM (total volume)(Gibco). Vero cells were washed with OPTI-MEM, and 150 μL of the transfection complex in OPTI-MEM was then added to each well containing 150 μL of OPTI-MEM media. Triplicate wells were tested for each condition. An additional control well with no transfection condition was prepared. Three hours post transfection, the media was removed. Each well was washed 1× with 200 μl 1×PBS containing 0.3% BSA, 10 mM HEPES and PS. Cells in each well were infected with WSN strain of Influenza virus at a multiplicity of infection (MOI) of 0.01 in 200 μL of infection media containing 0.3% BSA, 10 mM HEPES, PS and 4 μg/mL trypsin. The plate was incubated for one hour at 37° C. Unadsorbed virus was washed off with the 200 μL of infection media and discarded. Added gently to each well on the side of the well was 400 μl DMEM containing 0.3% BSA, 10 mM HEPES, PS and 4 μg/mL trypsin. The plate was incubated at 37° C., 5% CO₂, for 48 hours. From each well, 50 μL supernatant was tested in duplicate by TCID50 assays (Tissue-Culture Infective Dose 50, WHO protocol) and titers were estimated using Spearman and Karber formula.

Hemagglutination assays (HA) were used to quantify viruses and the extent of dsRNA-directed viral RNA interference in cell culture supernatants. Fifty microliters of 0.5% chicken red blood cells diluted with PBS were added to wells, and culture supernatants were added. Plates were incubated at room temperature for one hour. HA counts were used to calculate the viral titer in culture supernatants.

DX3148 is a negative control which is a scrambled random dicer substrate having a sense and antisense strand with the following nucleic acid sequence:

| | |
|---|---|
| DX3148 Sense Strand | |
| CUUCCUCUCUUUCUCUCCCUUGUdGdA-3′ | (SEQ ID NO: 111) |
| DX3148 Antisense Strand | |
| UCACAAGGGAGAGAAAGAGAGGAAGGA | (SEQ ID NO: 112) |

G1498 is a positive control non-dicer RNA targeting influenza RNA.

Reduction of WSN influenza viral titer in Vero cells by the influenza-specific dicer substrate peptide Conjugate 15 (dsCoP3072nfR3063) is shown in Table 15. The activity of Conjugate 15 (dsCoP3072nfR3063) was determined both in OPTI-MEM and in LIPOFECTAMINE 2000.

TABLE 15
Viral Titer Reduction for Influenza-Specific Dicer Substrates
Peptide Conjugate 15 (dsCoP3072nfR3063)

| | Concentration | | | |
|---|---|---|---|---|
| Treatment | 10 nM | 1 nM | 0.1 nM | 0.01 nM |
| Conjugate 15 | 6.26E+06 | 7.72E+06 | 8.04E+06 | 8.54E+06 |
| (dsCoP3072nfR3063) | | | | |
| in OPTI-MEM | | | | |
| Conjugate 15 | 5.62E+06 | 5.44E+06 | 6.26E+06 | 6.26E+06 |
| (dsCoP3072nfR3063) | | | | |
| in LIPOFECTAMINE | | | | |
| 2000 | | | | |
| DX3030 | 1.72E+07 | 1.83E+07 | 1.98E+07 | 8.54E+06 |
| (3063 dicer substrate | | | | |
| minus delivery peptide) | | | | |
| G1498 (non-dicer) | 6.26E+06 | 5.44E+06 | 1.57E+07 | 3.98E+06 |
| DX3148 (dicer substrate | 1.45E+07 | | | |
| negative control) | | | | |
| Qneg (dsRNA negative | 2.02E+07 | | | |
| control) | | | | |
| WSN Virus Control | 7.28E+07 | | | |

Reduction of WSN influenza viral titer in Vero cells by the influenza-specific dicer substrate peptide conjugate Conjugate 14 (dsCoP963nfR3063) is shown in Table 16. The activity of Conjugate 14 (dsCoP963nfR3063) was determined both in OPTIMEM and in LIPOFECTAMINE 2000.

TABLE 16
Viral Titer Reduction for Influenza-Specific Dicer Substrates
Peptide Conjugate 14 (dsCoP963nfR3063)

| | Concentration | | | |
|---|---|---|---|---|
| Treatment | 10 nM | 1 nM | 0.1 nM | 0.01 nM |
| Conjugate 14 | 4.38E+06 | 5.40E+06 | 5.63E+06 | 5.98E+06 |
| (dsCoP963nfR3063) | | | | |
| in OPTI-MEM | | | | |
| Conjugate 14 | 3.94E+06 | 3.81E+06 | 4.38E+06 | 4.38E+06 |
| (dsCoP963nfR3063) | | | | |
| in LIPOFECTAMINE | | | | |
| 2000 | | | | |
| DX3030 | 1.20E+07 | 1.28E+07 | 1.39E+07 | 5.98E+06 |
| (3063 dicer substrate | | | | |
| minus delivery peptide) | | | | |
| G1498 (non-dicer) | 4.38E+06 | 3.81E+06 | 1.10E+07 | 2.79E+06 |
| DX3148 (dicer substrate | 1.01E+07 | | | |
| negative control) | | | | |
| Qneg (dsRNA negative | 1.42E+07 | | | |
| control) | | | | |
| WSN Virus Control | 5.10E+07 | | | |

An additional data set showing reduction of WSN influenza viral titer in Vero cells by the influenza-specific dicer substrate peptide Conjugates 15 (dsCoP3072nfR3063), 14 (dsCoP963nfR3063) and 16 (dsCoP3073nfR3063) is shown in Table 17. Dharmacon buffer (SB) is 60 mM KCl, 6 mM HEPES-KOH, ph 7.5 and 0.2 mM MgCl₂). For comparison purposes, viral titer for the peptide dicer substrate complex DX3030 and PN963 (1:1) was determined.

TABLE 17
Viral Titer Reduction for Influenza-Specific Dicer Substrate Conjugates

| | Concentration | | | |
|---|---|---|---|---|
| Treatment | 10 nM | 1 nM | 0.1 nM | 0.01 nM |
| Conjugate 14 (dsCoP963nfR3063) | 4.39E+06 | 6.26E+06 | 8.54E+06 | 7.57E+06 |
| (G Stock) in SB | | | | |
| Conjugate 14 (dsCoP963nfR3063) | 8.54E+06 | 8.54E+06 | 7.08E+06 | 3.98E+06 |
| (M Stock) in SB | | | | |
| Conjugate 15 (dsCoP3072nfR3063) in SB | 7.08E+06 | 7.08E+06 | 1.11E+07 | 1.00E+07 |
| Conjugate 16 (dsCoP3073nfR3063) in SB | 3.16E+06 | 7.08E+06 | 1.00E+07 | 1.11E+07 |
| Conjugate 14 (dsCoP963nfR3063) in HEPES and | 2.44E+07 | 1.52E+07 | 3.52E+07 | 1.26E+07 |
| Dextrose | | | | |
| Conjugate 14 (dsCoP963nfR3063) Complexed | 8.54E+06 | 1.00E+07 | 1.00E+07 | 1.00E+07 |
| with PN3079 in HEPES and Dextrose | | | | |
| DX3030-PN963 Complex (1:1) in SB | 4.98E+07 | 2.24E+07 | 1.98E+07 | 6.26E+06 |
| DX3030-PN963 Complex (1:1) in HEPES and | 1.52E+07 | 6.26E+06 | 3.06E+07 | 8.54E+06 |
| Dextrose | | | | |
| DX3030 (3063 dicer RNA minus delivery peptide) | 1.00E+07 | 1.83E+07 | 1.52E+07 | 4.00E+07 |
| with LIPOFECTAMINE 2000 in SB | | | | |
| DX3030 (3063 dicer substrate minus delivery | 2.24E+07 | 7.00E+07 | 3.98E+07 | 1.43E+07 |
| peptide) in SB | | | | |
| G1498 (non-dicer) with LIPOFECTAMINE 2000 | 7.08E+06 | 7.08E+06 | 3.98E+06 | 1.42E+07 |
| in SB | | | | |
| PN963 in SB | 1.78E+07 | 1.00E+07 | 1.52E+07 | 1.52E+07 |
| DX3148 (dicer substrate negative control) with | 1.34E+07 | | | |
| LIPOFECTAMINE 2000 in SB | | | | |
| Qneg (dsRNA negative control) in SB | 2.02E+07 | | | |
| WSN Virus Control in SB | 1.84E+07 | | | |

The data in Tables 16, 17 and 18 show that influenza-specific dicer substrate peptide Conjugates 15 (dsCoP3072nfR3063), 14 (dsCoP963nfR306) 3 and 16 (dsCoP3073nfR3063) effectively reduced viral titer in Vero cells infected with WSN influenza virus when compared to the negative controls (e.g., DX3148 and Qneg). The DX3030 dicer substrate complexed with PN963 (1:1) did not reduce viral titer in Vero cells infected with WSN influenza virus.

To determine if the dicer substrate peptide conjugates reduced viral titer in Vero cells infected with WSN influenza virus at lower concentrations, an additional transfection was performed. Conjugates 15 (dsCoP3072nfR3063), 14 (dsCoP963nfR306) 3 and 16 (dsCoP3073nfR3063) were transfected at 0.1 pM, 1 pM, 0.01 nM, 0.1 nM, 1 nM, 10 nM and 100 nM. The cell culture, transfection and virus infection conditions were previously described. The data shown below in Table 18 is presented as the percent knockdown of viral titer relative to WSN virus control.

TABLE 18
Viral Titer Reduction for Influenza-Specific Dicer Substrate Conjugate

| | Concentration | | | | | | |
|---|---|---|---|---|---|---|---|
| Treatment | 100 nM | 10 nM | 1 nM | 0.1 nM | 0.01 nM | 1 pM | 0.1 pM |
| Conjugate 14 | 43.8% | 56.1% | 63.4% | 56.1% | 21.9% | 21.9% | 21.9% |
| (dsCoP963nfR3063) in SB | | | | | | | |
| Conjugate 15 | 43.8% | 34.2% | 34.2% | 21.9% | 29.2% | 0% | 0% |
| (dsCoP3072nfR3063) in SB | | | | | | | |
| Conjugate 16 | 75.3% | 63% | 52% | 43.8% | 21.9% | 21.9% | 14.6% |
| (dsCoP3073nfR3063) in SB | | | | | | | |
| DX3030 (3063 dicer RNA | 0% | | | 0% | | | 0% |
| minus delivery peptide) in SB | | | | | | | |
| DX3030 (3063 dicer substrate | 77.6% | | | 43.8% | | | 0% |
| minus delivery peptide) with | | | | | | | |
| LIPOFECTAMINE 2000 in SB | | | | | | | |
| dsCoP963nfRQNC | 0% | | | 0% | | | 0% |
| (Qneg conjugated to PN963) in | | | | | | | |
| SB | | | | | | | |
| PN3072 in SB | 0% | | | | | | |
| PN3073 in SB | 0% | | | | | | |
| DX3030-PN3072 Complex in | 56.1% | | | 14.6% | | | 0% |
| SB | | | | | | | |
| DX3030-PN3073 Complex in | 43.8% | | | 43.8% | | | 0% |
| SB | | | | | | | |
| dsCoP963nfR3063 in PBS | 21.9% | | | 21.9% | | | 14.6% |
| dsCoPN3073nfR3063 in PBS | 14.6% | | | 0% | | | 0% |
| dsCoPN3073nfR3063 in 10 mM | 0% | | | 0% | | | 0% |
| HEPES, 5% Dextrose | | | | | | | |
| Buffer, pH 7.4 | | | | | | | |
| WSN Virus Control in SB | 0% | | | | | | |

The data in Table 18 indicate that both Conjugates 14 (dsCoP963nfR3063) and 16 (dsCoP3073nfR3063) reduce WSN viral titer at 0.1 pM concentration.

Further, the data show that dicer substrate peptide Conjugates 15 (dsCoP3072nfR3063), 14 (dsCoP963nfR3063) and 16 (dsCoP3073nfR3063) provide their own formulation for delivery into cells in the absence of a transfection material such as LIPOFECTAMINE 2000. These data indicate that delivery peptides PN3072, PN963 and PN3073 exhibit intracellular delivery properties and additionally permit the conjugated double-stranded RNA to produce the response of RNA interference.

Also, Conjugates 15 (dsCoP3072nfR3063), 14 (dsCoP963nfR3063) and 16 (dsCoP3073nfR3063) exhibited greater knockdown activity of WSN viral titer in this assay than DX3030 (non-conjugate 3036 dsRNA dicer substrate). Thus, Conjugates 15 (dsCoP3072nfR3063), 14 (dsCoP963nfR3063) and 16 (dsCoP3073nfR3063) were found to be candidates for the treatment and/or prevention of influenza infection.

### EXAMPLE 55

### Degree of dsRNA Peptide Condensation at Varying N:P Ratios

This example demonstrates that higher ionic strength buffers reduce peptide self association and aggregate formation, thus permitting optimal intracellular delivery of a nucleic acid by a peptide and also permitting a conjugated dsRNA to produce the result of RNA interference.

The relative binding of various peptides to dicer substrates via a rapid screen is assessed by indirect measurement of the displacement of SYBR-gold nucleic acid binding dye. A buffered mixture of dsRNA, peptide and SYBR-gold was prepared in the measurement plate in duplicate such that the peptide and SYBR-gold dye undergoes simultaneous competitive binding of the siRNA. Two different ionic strength buffers, H/D (HEPES and dextrose, pH 7.4) and SB (Dharmacon, pH 7.4) were used to compare the effect of ionic strength on peptide/dsRNA condensation. In relative terms, SB is a higher ionic strength buffer than H/D buffer. The concentration of dsRNA was fixed at 10 μg/mL and is combined with a titration of each peptide ranging in a concentration that corresponds to a peptide:dsRNA charge ratio from 0 to 4. Since SYBR-gold dye only fluoresces when bound to dsRNA, peptide binding to the dsRNA inhibits binding of the dye and consequently reduces the fluorescence. Therefore, the amount of fluorescence correlates inversely to the binding of the peptide to the dsRNA.

Peptides were be suspended in Hyclone nuclease free water. Test dilutions of this peptide stock were made in sterile, Nuclease-free microcentifuge tubes at a 2× final concentration and buffered to pH 7.4 with 20 mM phosphate.

SYBR-gold nucleic acid binding dye stock, a 10,000× concentrate, was supplied by Invitrogen (Carlsbad, Calif.) and stored at −20° C. The concentrate was allowed to equilibrate to room temperature before diluting 1 to 100 in Hyclone nuclease free water, which was diluted 1 to 10 in the experimental plate for a final concentrate of 10× for the assay. This is the optimal dilution to achieve linear binding to siRNA duplex at a concentration range of up to 50 μg/mL concentration. For higher concentrations, the amount of dye to use will have to be optimized. The values used to generate the standard curve demonstrating linear binding of SYBR-gold to DX3030 dsRNA is shown in Table 19.

TABLE 19

Mean Fluorescence

| DX3030 Concentration | HEPES/Dextrose Buffer | SB Buffer |
|---|---|---|
| 0 μg/mL | 0 | 0 |
| 0.1 μg/mL | 5.4 | 39.8 |
| 0.25 μg/mL | 12.4 | 127.4 |
| 0.5 μg/mL | 38.1 | 240.4 |
| 1.0 μg/mL | 70.1 | 579.0 |
| 2.5 μg/mL | 242.9 | 1736.8 |
| 5.0 μg/mL | 523.7 | 3732.5 |
| 10 μg/mL | 8106.1 | 8212.0 |

“SB” is Dharmacon, pH 7.4 buffer.

The binding data presented as N/P ratio for dicer substrate DX3030 with PN963, PN3072 and PN3073, separately, in either HEPES/dextrose buffer (pH 7.4) or Dharmacon buffer (pH 7.4) is shown in Table 20.

TABLE 20
dsRNA Peptide Complex Fluorescence Values

| | DX3030- | | PN963 | | DX3030- | | PN3072 | | DX3030- | | PN3073 | |
|---|---|---|---|---|---|---|---|---|---|---|---|---|
| | PN963 | | (Peptide | | PN3072 | | (Peptide | | PN3073 | | (Peptide | |
| N/P | (Complex) | | alone) | | (Complex) | | alone) | | (Complex) | | alone) | |
| Ratio | H/D | SB | H/D | SB | H/D | SB | H/D | SB | H/D | SB | H/D | SB |
| 0 | 6330 | 7753 | 50 | 42 | 6233 | — | 46 | 42 | 6512 | 7908 | 45 | 41 |
| 0.02 | 5688 | 7839 | 44 | 44 | 6004 | 8013 | 46 | 45 | 6430 | 7586 | 44 | 49 |
| 0.1 | 4892 | 7379 | 53 | 45 | 5437 | 7824 | 45 | 43 | 5018 | 7050 | 43 | 41 |
| 0.2 | 4306 | 7140 | 54 | 45 | 3150 | 7677 | 45 | 43 | 3195 | 6342 | 42 | 37 |
| 0.4 | 3826 | 6818 | 44 | 47 | 1138 | 5657 | 45 | 43 | 1201 | 5029 | 40 | 39 |
| 0.8 | 2861 | 6475 | 46 | 47 | 939 | 5307 | 44 | 41 | 1645 | 3399 | 39 | 37 |
| 1 | 2343 | 7132 | 43 | 48 | 1012 | 5413 | 44 | 41 | 2336 | 2436 | 39 | 37 |
| 2 | 829 | 6552 | 43 | 50 | 983 | 4976 | 44 | 38 | 2574 | 3032 | 38 | 36 |
| 4 | 971 | 4912 | 48 | 60 | 1290 | 4456 | 42 | 37 | 2267 | 2975 | 38 | 37 |

“H/D” is HEPES/dextrose, pH 7.4 buffer and
“SB” is Dharmacon, pH 7.4 buffer.

The data in Table 20 indicate minimal complex formation of PN963 with dicer substrate DX3030 in SB buffer (higher ionic strength) and a more stable complex in the H/D buffer (lower ionic strength). Similar results are observed with PN3072. These data indicate that delivery peptides PN963, PN3072, and to a lesser degree PN3073, in relatively high ionic strength buffer (e.g., SB buffer) do not associate with dicer substrate RNA.

### EXAMPLE 56

### Viral Titer Reduction in Human-Derived Tracheal/Bronchial Epithelial Cells by Influenza-Specific Dicer Substrate Peptide Conjugates

Reduction of WSN influenza NP RNA levels in human-derived tracheal/bronchial epithelial cell model system (MATEK EPIAIRWAY system) by dicer substrate peptide conjugates was determined. Briefly, the epithelial cells were plated on a 96-well plated at approximately 160,000 to 200,000/well and permitted to recover overnight in basal media (phenol red-free and hydrocortisone-free Dulbecco's Modified Eagle's Media (DMEM)) at 37° C./5% CO₂. Mucus was removed and the cells were washed once with 100 μL PBS. Transfection was performed by mixing 25 μL Air-100 media (MatTek) with 0.5 μL of each RNA or dicer substrate peptide conjugate at 100 nM, 10 nM, 1 nM or 0.1 nM, and adding the mixture to cells in each well. Transfecture mixture was incubated with cells for four hours and then removed. Cells were washed once with 100 μL PBS followed by infection with WSN virus.

Cells were infected with WSN virus as follows: First, each well was incubated for one hour with 0.5 μL influenza virus (100 pfu; MOI=0.0004) and 25 μL PBS containing 0.3% BSA. To each well, 75 μL Air-100 media (MatTek) containing 0.3% BSA and 4 μg/mL trypsin was added. After incubation with virus and additional media, the media was removed and fresh basal media added. RNA was isolated from cell 48 hours post infection with a TURBOCAPTURE kit (Qiagen).

RNA levels were quantified with SYBR Green qRT-PCR kit (Qiagen). The manufacturer's protocol was followed.

Influenza viral NP RNA reduction in human-derived tracheal/bronchial epithelial cells by the influenza-specific dicer substrate peptide Conjugate 14 (dsCoP963nfR3063) and Conjugate 15 (dsCoP3073nfR3063) is shown in Table 21. Influenza NP RNA reduction for dicer substrate peptide conjugates is expressed as percent relative to dsCoP963nfRQNC (Qneg RNA conjugated to delivery peptide PN963).

TABLE 21
Viral NP RNA Reduction for Influenza-Specific
Dicer Substrate Conjugates

| | Concentration | | | |
|---|---|---|---|---|
| Treatment | 100 nM | 10 nM | 1 nM | 0.1 nM |
| Conjugate 14 (dsCoP963nfR3063) | 0% | 0% | 36% | 79.2% |
| in SB | | | | |
| Conjugate 16 (dsCoP3073nfR3063) | 0% | 9.5% | 35% | 41.6% |
| in SB | | | | |
| DX3030 (3063 dicer RNA minus | 0% | | | |
| delivery peptide) with | | | | |
| LIPOFECTAMINE 2000 in SB | | | | |
| DX3030 (3063 dicer substrate | 0% | | | |
| minus delivery peptide) in SB | | | | |
| G1498 (non-dicer) with | 0% | | | |
| LIPOFECTAMINE 2000 | | | | |
| in SB | | | | |
| DX3148 (dicer substrate negative | 0% | | | |
| control) with LIPOFECTAMINE | | | | |
| 2000 in SB | | | | |

### EXAMPLE 57

### A Dicer Substrate Peptide Conjugate Exhibits Influenza Reduction in Vero Cells at Concentrations as low as 10 Femtomolar

Viral titer was determined for dicer substrate peptide Conjugate 14, dsCoP963nfR3063, in Vero cells infected with WSN influenza virus at transfected concentrations that were varied over six decades from 10 nM to 10 fM. Table 22 shows the percent knockdown of viral titer relative to WSN virus control.

TABLE 22
Dicer Substrate Conjugate Viral Titer Percent Knockdown Relative to Virus Control

| | Concentration (nM) | | | | | | |
|---|---|---|---|---|---|---|---|
| Dicer RNA | 10 | 1 | 0.1 | 0.01 | 0.001 | 0.0001 | 0.00001 |
| Conjugate 14 | 73.6% | 81.3% | 63.2% | 73.6% | 79.3% | 63.2% | 66.7% |
| (dsCoP963nfR3063) | | | | | | | |
| DX3030 | 81.3% | — | — | — | — | — | — |
| (in Lipofectamine ™ 2000) | | | | | | | |
| Qneg | 0.0% | — | — | — | — | — | — |

The data in Table 22 show that Conjugate 14 (dsCoP963nfR3063), when transfected at a concentration as low as 10 femtomolar, without any additional transfection agent, effectively reduced WSN viral titer in Vero cells. These data show that dicer substrate peptide Conjugate 14 provided its own formulation for delivery into cells in the absence of a separate or additional transfection material, while providing a remarkably high level of anti-influenza activity.

Lastly, the anti-viral activity of this dicer substrate peptide conjugate persisted after three freeze-thaw cycles down to −30° C.

What is claimed is:

1. A dicer substrate peptide conjugate comprising a double stranded ribonucleic acid (dsRNA) having a sense strand and an antisense strand and a double-stranded region of from 25 to 30 base pairs, and a peptide comprising SEQ ID NO:32 or a variant thereof, wherein the dsRNA is conjugated to the peptide.

2. The dicer substrate peptide conjugate of claim 1, wherein the variant is selected from the group consisting of SEQ ID NO:46 PN3846, SEQ ID NO:47 PN3847, SEQ ID NO:48 PN3848, SEQ ID NO:52 PN3889, SEQ ID NO:50 PN3885, SEQ ID NO:54 PN3980, SEQ ID NO:55 PN3981, SEQ ID NO: 51 PN3886, and SEQ ID NO: 53 PN3948.

3. A dicer substrate peptide conjugate comprising a double stranded ribonucleic acid (dsRNA) having a sense strand and an antisense strand and a double-stranded region of from 25 to 30 base pairs, and a peptide comprising SEQ ID NO: 34 or a variant thereof, wherein the dsRNA is conjugated to the peptide.

4. A dicer substrate peptide conjugate comprising a double stranded ribonucleic acid (dsRNA) having a sense strand and an antisense strand and a double-stranded region of from 25 to 30 base pairs, and a peptide comprising SEQ ID NO: 35 or a variant thereof, wherein the dsRNA is conjugated to the peptide.

5. A dicer substrate peptide conjugate comprising a double stranded ribonucleic acid (dsRNA) having a sense strand and an antisense strand and a double-stranded region of from 25 to 30 base pairs, and a peptide comprising SEQ ID NO: 38 or a variant thereof, wherein the dsRNA is conjugated to the peptide.

6. A dicer substrate peptide conjugate comprising a double stranded ribonucleic acid (dsRNA) having a sense strand and an antisense strand and a double-stranded region of from 25 to 30 base pairs, and a peptide comprising a PreS2 translocation motif or a variant thereof, wherein the dsRNA is conjugated to the peptide.

7. The dicer substrate peptide conjugate of claim 6, wherein the variant is selected from the group consisting of SEQ ID NO: 34 and SEQ ID NO: 35.

8. A dicer substrate peptide conjugate comprising a double stranded ribonucleic acid (dsRNA) having a sense strand and an antisense strand and a double-stranded region of from 25 to 30 base pairs, and a peptide comprising SEQ ID NO: 45 or a variant thereof, wherein the dsRNA is conjugated to the peptide.

9. A dicer substrate peptide conjugate comprising a double stranded ribonucleic acid (dsRNA) having a sense strand and an antisense strand and a double-stranded region of from 25 to 30 base pairs, and a chimeric peptide comprising penatratin or a variant thereof, and a polyarginine comprising at least five arginine residues, wherein the dsRNA is conjugated to the chimeric peptide.

10. The dicer substrate peptide conjugate of claim 9, wherein the chimeric peptide is selected from the group consisting of SEQ ID NO: 56 and SEQ ID NO: 57.

11. A dicer substrate peptide conjugate comprising a double stranded ribonucleic acid (dsRNA) having a sense strand and an antisense strand and a double-stranded region of from 25 to 30 base pairs, and a peptide comprising SEQ ID NO: 58 or a variant thereof, wherein the dsRNA is conjugated to the peptide.

12. A dicer substrate peptide conjugate comprising a double stranded ribonucleic acid (dsRNA) having a sense strand and an antisense strand and a double-stranded region of from 25 to 30 base pairs, and a peptide comprising SEQ ID NO: 59 or a variant thereof, wherein the dsRNA is conjugated to the peptide.

13. A dicer substrate peptide conjugate comprising a double stranded ribonucleic acid (dsRNA) having a sense strand and an antisense strand and a double-stranded region of from 25 to 30 base pairs, and a peptide comprising SEQ ID NO: 60 or a variant thereof, wherein the dsRNA is conjugated to the peptide.

14. A dicer substrate peptide conjugate comprising a double stranded ribonucleic acid (dsRNA) having a sense strand and an antisense strand and a double-stranded region of from 25 to 30 base pairs, and a peptide comprising SEQ ID NO: 61 or a variant thereof, wherein the dsRNA is conjugated to the peptide.

15. A dicer substrate peptide conjugate comprising a double stranded ribonucleic acid (dsRNA) having a sense strand and an antisense strand and a double-stranded region of from 25 to 30 base pairs, and a peptide comprising SEQ ID NO: 62 or a variant thereof, wherein the dsRNA is conjugated to the peptide.

16. A dicer substrate peptide conjugate comprising a double stranded ribonucleic acid (dsRNA) having a sense strand and an antisense strand and a double-stranded region of from 25 to 30 base pairs, and a peptide comprising from about 5 to about 100 amino acids, wherein the peptide comprises at least two positively charge amino acid residues, and wherein the dsRNA is conjugated to the peptide.

17. A pharmaceutical composition comprising a dicer substrate peptide conjugate of claim 1 and one or more carriers.

18. A method for treating influenza in an animal comprising administering an effective amount of a dicer substrate peptide conjugate of claim 1 to the animal.

19. A method for treating inflammation associated with TNF-alpha in an animal comprising administering an effective amount of a dicer substrate peptide conjugate of claim 1 to the animal.

20. The method of claim 19, wherein the inflammation occurs in arthritis.

21. The method of claim 19, wherein the inflammation occurs in psoriasis.

22. A method for inhibiting expression of a TNF-alpha gene in an animal comprising administering an inhibiting amount of a pharmaceutical composition of a dicer substrate peptide conjugate of claim 1 to the animal.


SEQUENCE LISTING (113 sequences)

SEQ ID NO: 1 - protein, 7 aa, Human immunodeficiency virus
KRRQRRR

SEQ ID NO: 2 - protein, 16 aa, Artificial Sequence
  Description of Artificial Sequence Synthetic peptide
RQIKIWFQNRRMKWKK

SEQ ID NO: 3 - protein, 34 aa, Human herpesvirus
DAATATRGRSAASRPTERPRAPARSASRPRRPVD

SEQ ID NO: 4 - protein, 16 aa, Artificial Sequence
  Description of Artificial Sequence Synthetic peptide
AAVALLPAVLLALLAP

SEQ ID NO: 5 - protein, 16 aa, Artificial Sequence
  Description of Artificial Sequence Synthetic peptide
AAVLLPVLLPVLLAAP

SEQ ID NO: 6 - protein, 15 aa, Homo sapiens
VTVLALGALAGVGVG

SEQ ID NO: 7 - protein, 17 aa, Human immunodeficiency virus
GALFLGWLGAAGSTMGA

SEQ ID NO: 8 - protein, 17 aa, Caiman crocodylus
MGLGLHLLVLAAALQGA

SEQ ID NO: 9 - protein, 24 aa, Homo sapiens
LGTYTQDFNKFHTFPQTAIGVGAP

SEQ ID NO: 10 - protein, 26 aa, Artificial Sequence
  Description of Artificial Sequence Synthetic peptide
GWTLNSAGYLLKINLKALAALAKKIL

SEQ ID NO: 11 - protein, 16 aa, Artificial Sequence
  Description of Artificial Sequence Synthetic peptide
TPPKKKRKVEDPKKKK

SEQ ID NO: 12 - protein, 7 aa, Artificial Sequence
  Description of Artificial Sequence Synthetic peptide
RRRRRRR

SEQ ID NO: 13 - protein, 18 aa, Artificial Sequence
  Description of Artificial Sequence Synthetic peptide
KLALKLALKALKAALKLA

SEQ ID NO: 14 - protein, 16 aa, Influenza virus
GLFGAIAGFIENGWEG

SEQ ID NO: 15 - protein, 16 aa, Human parainfluenza virus
FFGAVIGTIALGVATA

SEQ ID NO: 16 - protein, 16 aa, Respiratory syncytial virus
FLGFLLGVGSAIASGV

SEQ ID NO: 17 - protein, 16 aa, Human immunodeficiency virus
GVFVLGFLGFLATAGS

SEQ ID NO: 18 - protein, 16 aa, Ebola virus
GAAIGLAWIPYFGPAA

SEQ ID NO: 19 - protein, 56 aa, Artificial Sequence
  Description of Artificial Sequence Synthetic peptide
ACTCPYCKDSEGRGSGDPGKKKQHICHIQGCGKVYGKTSHLRAHLRWHTGERPFMC

SEQ ID NO: 20 - protein, 54 aa, Artificial Sequence
  Description of Artificial Sequence Synthetic peptide
ACTCPNCKDGEKRSGEQGKKKHVCHIPDCGKTFRKTSLLRAHVRLHTGERPFVC

SEQ ID NO: 21 - protein, 55 aa, Artificial Sequence
  Description of Artificial Sequence Synthetic peptide
ACTCPNCKEGGGRGTNLGKKKQHICHIPGCGKVYGKTSHLRAHLRWHSGERPFVC

SEQ ID NO: 22 - protein, 56 aa, Artificial Sequence
  Description of Artificial Sequence Synthetic peptide
ACSCPNCREGEGRGSNEPGKKKQHICHIEGCGKVYGKTSHLRAHLRWHTGERPFIC

SEQ ID NO: 23 - protein, 60 aa, Drosophila melanogaster
RCTCPNCTNEMSGLPPIVGPDERGRKQHICHIPGCERLYGKASHLKTHLRWHTGERPFLC

SEQ ID NO: 24 - protein, 58 aa, Drosophila melanogaster
TCDCPNCQEAERLGPAGVHLRKKNIHSCHIPGCGKVYGKTSHLKAHLRWHTGERPFVC

SEQ ID NO: 25 - protein, 53 aa, Caenorhabditis elegans
RCTCPNCKAIKHGDRGSQHTHLCSVPGCGKTYKKTSHLRAHLRKHTGDRPFVC

SEQ ID NO: 26 - protein, 56 aa, Artificial Sequence
  Description of Artificial Sequence Synthetic peptide
PQISLKKKIFFFIFSNFRGDGKSRIHICHLCNKTYGKTSHLRAHLRGHAGNKPFAC

SEQ ID NO: 27 - protein, 36 aa, Artificial Sequence
  Description of Artificial Sequence Synthetic peptide
KGSKKAVTKAQKKDGKKRKRSRKESYSVYVYKVLKQ

SEQ ID NO: 28 - protein, 36 aa, Artificial Sequence
  Description of Artificial Sequence Synthetic peptide
KGSKKAVTKAQKKEGKKRKRSRKESYSVYVYKVLKQ

SEQ ID NO: 29 - protein, 27 aa, Artificial Sequence
  Description of Artificial Sequence Synthetic peptide
AQKKEGKKRKRSRKESYSVYVYKVLKQ

SEQ ID NO: 30 - protein, 18 aa, Artificial Sequence
  Description of Artificial Sequence Synthetic peptide
KRSRKESYSVYVYKVLKQ

SEQ ID NO: 31 - protein, 18 aa, Artificial Sequence
  Description of Artificial Sequence Synthetic peptide
KRSRKESYSVYVYKVLKQ

SEQ ID NO: 32 - protein, 13 aa, Artificial Sequence
  Description of Artificial Sequence Synthetic peptide
ESYSVYVYKVLKQ

SEQ ID NO: 33 - protein, 6 aa, Artificial Sequence
  Description of Artificial Sequence Synthetic peptide
YKVLKQ

SEQ ID NO: 34 - protein, 15 aa, Artificial Sequence
  Description of Artificial Sequence Synthetic peptide
RVIRWFQNKRSKDKK

SEQ ID NO: 35 - protein, 27 aa, Artificial Sequence
  Description of Artificial Sequence Synthetic peptide
GALFLGFLGAAGSTMGAWSQPKSKRKV

SEQ ID NO: 36 - protein, 16 aa, Artificial Sequence
  Description of Artificial Sequence Synthetic peptide
RQIKIWFQNRRMKWKK

SEQ ID NO: 37 - protein, 16 aa, Artificial Sequence
  Description of Artificial Sequence Synthetic peptide
RQIKIWFQNRRMKWKK

SEQ ID NO: 38 - protein, 26 aa, Artificial Sequence
  Description of Artificial Sequence Synthetic peptide
GWTLNSAGYLLKINLKALAALAKKIL

SEQ ID NO: 39 - protein, 21 aa, Artificial Sequence
  Description of Artificial Sequence Synthetic peptide
LLNQLAGRMIPKWSQKSKRKV

SEQ ID NO: 40 - protein, 20 aa, Artificial Sequence
  Description of Artificial Sequence Synthetic peptide
TLDHVLDHVQTWSQKSKRKV

SEQ ID NO: 41 - protein, 23 aa, Artificial Sequence
  Description of Artificial Sequence Synthetic peptide
SYFILRRRRKRFPYFFTDVRVAA

SEQ ID NO: 42 - protein, 10 aa, Artificial Sequence
  Description of Artificial Sequence Synthetic peptide
RRRRRRRRRR

SEQ ID NO: 43 - protein, 8 aa, Artificial Sequence
  Description of Artificial Sequence Synthetic peptide
RRRRRRRR

SEQ ID NO: 44 - protein, 28 aa, Artificial Sequence
  Description of Artificial Sequence Synthetic peptide
KETWWETWWTEWSQPGRKKRRQRRRPPQ

SEQ ID NO: 45 - protein, 17 aa, Artificial Sequence
  Description of Artificial Sequence Synthetic peptide
GRPRESGKKRKRKRLKP

SEQ ID NO: 46 - protein, 13 aa, Artificial Sequence
  Description of Artificial Sequence Synthetic peptide
KSYSVYVYKVLKQ

SEQ ID NO: 47 - protein, 13 aa, Artificial Sequence
  Description of Artificial Sequence Synthetic peptide
ESYSVYVYRVLRQ

SEQ ID NO: 48 - protein, 13 aa, Artificial Sequence
  Description of Artificial Sequence Synthetic peptide
RSYSVYVYRVLRQ

SEQ ID NO: 49 - protein, 13 aa, Artificial Sequence
  Description of Artificial Sequence Synthetic peptide
QKLVKYVYVSYSE

SEQ ID NO: 50 - protein, 13 aa, Artificial Sequence
  Description of Artificial Sequence Synthetic peptide
ESYSVYVYKVLKQ

SEQ ID NO: 51 - protein, 13 aa, Artificial Sequence
  Description of Artificial Sequence Synthetic peptide
ASYSVYVYAVLAQ

SEQ ID NO: 52 - protein, 13 aa, Artificial Sequence
  Description of Artificial Sequence Synthetic peptide
QKLVKYVYVSYSE

SEQ ID NO: 53 - protein, 13 aa, Artificial Sequence
  Description of Artificial Sequence Synthetic peptide
ESYSVYVYKVLKQ

SEQ ID NO: 54 - protein, 19 aa, Artificial Sequence
  Description of Artificial Sequence Synthetic peptide
RRRRRRESYSVYVYKVLKQ

SEQ ID NO: 55 - protein, 19 aa, Artificial Sequence
  Description of Artificial Sequence Synthetic peptide
ESYSVYVYKVLKQRRRRRR

SEQ ID NO: 56 - protein, 22 aa, Artificial Sequence
  Description of Artificial Sequence Synthetic peptide
RRRRRRRQIKIWFQNRRMKWKK

SEQ ID NO: 57 - protein, 22 aa, Artificial Sequence
  Description of Artificial Sequence Synthetic peptide
RQIKIWFQNRRMKWKKRRRRRR

SEQ ID NO: 58 - protein, 17 aa, Artificial Sequence
  Description of Artificial Sequence Synthetic peptide
KTKIESLKEHGRRRRRR

SEQ ID NO: 59 - protein, 31 aa, Artificial Sequence
  Description of Artificial Sequence Synthetic peptide
MDVNPTLLFLKVPAQNAISTTFPYTRRRRRR

SEQ ID NO: 60 - protein, 26 aa, Artificial Sequence
  Description of Artificial Sequence Synthetic peptide
GLFEALLELLESLWELLLEARRRRRR

SEQ ID NO: 61 - protein, 18 aa, Artificial Sequence
  Description of Artificial Sequence Synthetic peptide
LLNQLAGRMIPKRRRRRR

SEQ ID NO: 62 - protein, 17 aa, Artificial Sequence
  Description of Artificial Sequence Synthetic peptide
TLDHVLDHVQTRRRRRR

SEQ ID NO: 63 - protein, 26 aa, Artificial Sequence
  Description of Artificial Sequence Synthetic peptide
GLFGAIAGFIENGWEGMIDGRRRRRR

SEQ ID NO: 64 - protein, 17 aa, Artificial Sequence
  Description of Artificial Sequence Synthetic peptide
KETWWETWWTERRRRRR

SEQ ID NO: 65 - protein, 16 aa, Artificial Sequence
  Description of Artificial Sequence Synthetic peptide
HHHHHHHHHHRRRRRR

SEQ ID NO: 66 - protein, 22 aa, Artificial Sequence
  Description of Artificial Sequence  Synthetic peptide
AAVALLPAVLLALLAPRRRRRR

SEQ ID NO: 67 - DNA, 25 bp, Artificial Sequence
  Description of Combined DNA/RNA Molecule Synthetic oligonucleotide
cccaugugcuccucacccacaccat

SEQ ID NO: 68 - RNA, 27 nt, Artificial Sequence
  Description of Artificial Sequence Synthetic oligonucleotide
auggugugggugaggagcacaugggug

SEQ ID NO: 69 - RNA, 27 nt, Artificial Sequence
  Description of Artificial Sequence Synthetic oligonucleotide
gccuguaccucaucuacucccaggucc

SEQ ID NO: 70 - RNA, 29 nt, Artificial Sequence
  Description of Artificial Sequence Synthetic oligonucleotide
gguccugggaguagaugagguacaggcuu

SEQ ID NO: 71 - DNA, 25 bp, Artificial Sequence
  Description of Combined DNA/RNA Molecule Synthetic oligonucleotide
ggaucuuauuucuucggagacaatg

SEQ ID NO: 72 - RNA, 27 nt, Artificial Sequence
  Description of Artificial Sequence Synthetic oligonucleotide
cauugucuccgaagaaauaagauccuu

SEQ ID NO: 73 - DNA, 25 bp, Artificial Sequence
  Description of Combined DNA/RNA Molecule Synthetic oligonucleotide
gggucggaacccaagcuuagaactt

SEQ ID NO: 74 - RNA, 27 nt, Artificial Sequence
  Description of Artificial Sequence Synthetic oligonucleotide
aaguucuaagcuuggguuccgacccua

SEQ ID NO: 75 - RNA, 25 nt, Artificial Sequence
  Description of Artificial Sequence Synthetic oligonucleotide
gacucagcgcugagaucaaucggcc

SEQ ID NO: 76 - RNA, 27 nt, Artificial Sequence
  Description of Artificial Sequence Synthetic oligonucleotide
ggccgauugaucucagcgcugagucgg

SEQ ID NO: 77 - DNA, 25 bp, Artificial Sequence
  Description of Combined DNA/RNA Molecule Synthetic oligonucleotide
gucucagccucuucucauuccugct

SEQ ID NO: 78 - RNA, 27 nt, Artificial Sequence
  Description of Artificial Sequence Synthetic oligonucleotide
agcaggaaugagaagaggcugagacau

SEQ ID NO: 79 - DNA, 25 bp, Artificial Sequence
  Description of Combined DNA/RNA Molecule Synthetic oligonucleotide
cuacacaaaucagcgauuuccaugt

SEQ ID NO: 80 - RNA, 27 nt, Artificial Sequence
  Description of Artificial Sequence Synthetic oligonucleotide
acauggaaaucgcugauuuguguaguc

SEQ ID NO: 81 - RNA, 25 nt, Artificial Sequence
  Description of Artificial Sequence Synthetic oligonucleotide
ggaaagacuguuccaaaaacagugg

SEQ ID NO: 82 - RNA, 27 nt, Artificial Sequence
  Description of Artificial Sequence Synthetic oligonucleotide
ccacuguuuuuggaacagucuuuccuu

SEQ ID NO: 83 - DNA, 25 bp, Artificial Sequence
  Description of Combined DNA/RNA Molecule Synthetic oligonucleotide
cccaugugcuccucacccacaccat

SEQ ID NO: 84 - RNA, 27 nt, Artificial Sequence
  Description of Artificial Sequence Synthetic oligonucleotide
auggugugggugaggagcacaugggug

SEQ ID NO: 85 - RNA, 29 nt, Artificial Sequence
  Description of Artificial Sequence Synthetic oligonucleotide
gguccugggaguagaugagguacaggcuu

SEQ ID NO: 86 - RNA, 27 nt, Artificial Sequence
  Description of Artificial Sequence Synthetic oligonucleotide
gccuguaccucaucuacucccaggucc

SEQ ID NO: 87 - DNA, 25 bp, Artificial Sequence
  Description of Combined DNA/RNA Molecule Synthetic oligonucleotide
cccaugugcuccucacccacaccat

SEQ ID NO: 88 - DNA, 25 bp, Artificial Sequence
  Description of Combined DNA/RNA Molecule Synthetic oligonucleotide
gggucggaacccaagcuuagaactt

SEQ ID NO: 89 - DNA, 25 bp, Artificial Sequence
  Description of Combined DNA/RNA Molecule Synthetic oligonucleotide
gacucagcgcugagaucaaucggcc

SEQ ID NO: 90 - DNA, 25 bp, Artificial Sequence
  Description of Combined DNA/RNA Molecule Synthetic oligonucleotide
gucucagccucuucucauuccugct

SEQ ID NO: 91 - DNA, 25 bp, Artificial Sequence
  Description of Combined DNA/RNA Molecule Synthetic oligonucleotide
gucucagccucuucucauuccugct

SEQ ID NO: 92 - DNA, 25 bp, Artificial Sequence
  Description of Combined DNA/RNA Molecule Synthetic oligonucleotide
cccaugugcuccucacccacaccat

SEQ ID NO: 93 - DNA, 25 bp, Artificial Sequence
  Description of Combined DNA/RNA Molecule Synthetic oligonucleotide
cccaugugcuccucacccacaccat

SEQ ID NO: 94 - DNA, 25 bp, Artificial Sequence
  Description of Combined DNA/RNA Molecule Synthetic oligonucleotide
cccaugugcuccucacccacaccat

SEQ ID NO: 95 - DNA, 25 bp, Artificial Sequence
  Description of Combined DNA/RNA Molecule Synthetic oligonucleotide
cccaugugcuccucacccacaccat

SEQ ID NO: 96 - protein, 10 aa, Artificial Sequence
  Description of Artificial Sequence Synthetic peptide
RRRRRRRRRR

SEQ ID NO: 97 - RNA, 21 nt, Artificial Sequence
  Description of Artificial Sequence Synthetic oligonucleotide
gccuguaccucaucuacucuu

SEQ ID NO: 98 - RNA, 21 nt, Artificial Sequence
  Description of Artificial Sequence Synthetic oligonucleotide
gaguagaugagguacaggcuu

SEQ ID NO: 99 - RNA, 25 nt, Artificial Sequence
  Description of Artificial Sequence Synthetic oligonucleotide
gccucuucuccuuccugaucguggc

SEQ ID NO: 100 - RNA, 27 nt, Artificial Sequence
  Description of Artificial Sequence Synthetic oligonucleotide
gccacgaucaggaaggagaagaggcug

SEQ ID NO: 101 - RNA, 25 nt, Artificial Sequence
  Description of Artificial Sequence Synthetic oligonucleotide
gccugcugcacuuuggagugaucgg

SEQ ID NO: 102 - RNA, 27 nt, Artificial Sequence
  Description of Artificial Sequence Synthetic oligonucleotide
ccgaucacuccaaagugcagcaggcag

SEQ ID NO: 103 - DNA, 25 bp, Artificial Sequence
  Description of Combined DNA/RNA Molecule Synthetic oligonucleotide
accucaucuacucccagguccuctt

SEQ ID NO: 104 - RNA, 27 nt, Artificial Sequence
  Description of Artificial Sequence Synthetic oligonucleotide
aagaggaccugggaguagaugagguac

SEQ ID NO: 105 - DNA, 21 bp, Artificial Sequence
  Description of Combined DNA/RNA Molecule Synthetic oligonucleotide
uucuccgaacgugucacgutt

SEQ ID NO: 106 - DNA, 21 bp, Artificial Sequence
  Description of Combined DNA/RNA Molecule Synthetic oligonucleotide
acgugacacguucggagaatt

SEQ ID NO: 107 - DNA, 21 bp, Artificial Sequence
  Description of Combined DNA/RNA Molecule Synthetic oligonucleotide
ggaucuuauuucuucggagtt

SEQ ID NO: 108 - DNA, 21 bp, Artificial Sequence
  Description of Combined DNA/RNA Molecule Synthetic oligonucleotide
cuccgaagaaauaagaucctt

SEQ ID NO: 109 - RNA, 25 nt, Artificial Sequence
  Description of Artificial Sequence Synthetic oligonucleotide
cuuccucucuuucucucccuuguga

SEQ ID NO: 110 - RNA, 27 nt, Artificial Sequence
  Description of Artificial Sequence Synthetic oligonucleotide
ucacaagggagagaaagagaggaagga

SEQ ID NO: 111 - RNA, 25 nt, Artificial Sequence
  Description of Artificial Sequence Synthetic oligonucleotide
cuuccucucuuucucucccuuguga

SEQ ID NO: 112 - RNA, 27 nt, Artificial Sequence
  Description of Artificial Sequence Synthetic oligonucleotide
ucacaagggagagaaagagaggaagga

SEQ ID NO: 113 - protein, 23 aa, Artificial Sequence
  Description of Artificial Sequence Synthetic peptide
CXXXXCXXXXXXXXXXXXHXXXH